(19) United States
(12) Patent Application Publication
(10) Pub. No.: US 2026/0028628 A1
(43) Pub. Date: Jan. 29, 2026

(54) COMPOSITIONS AND METHODS FOR INHIBITION OF EXPRESSION OF INHIBIN SUBUNIT BETA E (INHBE) GENES

(71) Applicant: BASECURE THERAPEUTICS LLC, Dover, DE (US)

(72) Inventors: Jing YANG, Dover, DE (US); Kevin J. KIM, Dover, DE (US); Saul Martinez MONTERO, Dover, DE (US)

(21) Appl. No.: 19/245,022

(22) Filed: Jun. 20, 2025

Related U.S. Application Data
(63) Continuation of application No. PCT/US2024/019416, filed on Mar. 11, 2024.
(60) Provisional application No. 63/489,325, filed on Mar. 9, 2023.

(51) Int. Cl. C12N 15/113

(52) U.S. Cl. CPC C12N 15/1136; C12N 2310/14; C12N 2310/315; C12N 2310/321; C12N 2310/351

(57) ABSTRACT

The disclosure relates to double-stranded ribonucleic acid (dsRNA) targeting an INHBE gene, and methods of using the dsRNA to inhibit expression of INHBE.

### CROSS-REFERENCE

This application is a continuation application of International Patent Application No. PCT/US2024/019416, which claims the benefit of and priority to U.S. Provisional Patent Application No. 63/489,325, filed Mar. 9, 2023, the disclosures of which are incorporated herein by reference in their entireties.

### SEQUENCE LISTING

The instant application contains a Sequence Listing which has been submitted electronically in XML format and is hereby incorporated by reference in its entirety. Said XML copy, created on Jun. 20, 2025, is named 139866-5001-US-Sequence Listing.xml, and is 2,433,336 bytes in size.

### FIELD OF THE INVENTION

The disclosure relates to double-stranded ribonucleic acid (dsRNA) targeting INHBE genes, and methods of using the dsRNA to inhibit expression of INHBE in a cell.

### SUMMARY

The present disclosure is based, in part, upon the development of double-stranded ribonucleic acid (dsRNA) targeting Inhibin Subunit Beta E (INHBE) genes, pharmaceutical compositions comprising the dsRNAs targeting INHBE genes and methods of using the dsRNA to inhibit expression of INHBE in a cell.

In some aspects, the disclosure provides for a double-stranded ribonucleic acid (dsRNA) for inhibiting expression of INHBE comprising a sense strand and an antisense strand each 15 to 30 nucleotides in length, wherein: (a) the antisense strand comprises a sequence that is at least 70% or 80% identical to the sequence of SEQ ID NO: 598, and the sense strand comprises a sequence that is at least 70% or 80% identical to the sequence of SEQ ID NO: 589; (b) the antisense strand comprises a sequence that is at least 70% or 80% identical to the sequence of SEQ ID NO: 599, and the sense strand comprises a sequence that is at least 70% or 80% identical to the sequence of SEQ ID NO: 590; (c) the antisense strand comprises a sequence that is at least 70% or 80% identical to the sequence of SEQ ID NO: 600, and the sense strand comprises a sequence that is at least 70% or 80% identical to the sequence of SEQ ID NO: 591; (d) the antisense strand comprises a sequence that is at least 70% or 80% identical to the sequence of SEQ ID NO: 601, and the sense strand comprises a sequence that is at least 70% or 80% identical to the sequence of SEQ ID NO: 592; (e) the antisense strand comprises a sequence that is at least 70% or 80% identical to the sequence of SEQ ID NO: 602, and the sense strand comprises a sequence that is at least 70% or 80% identical to the sequence of SEQ ID NO: 593; (f) the antisense strand comprises a sequence that is at least 70% or 80% identical to the sequence of SEQ ID NO: 603, and the sense strand comprises a sequence that is at least 70% or 80% identical to the sequence of SEQ ID NO: 594; (g) the antisense strand comprises a sequence that is at least 70% or 80% identical to the sequence of SEQ ID NO: 604, and the sense strand comprises a sequence that is at least 70% or 80% identical to the sequence of SEQ ID NO: 595; (h) the antisense strand comprises a sequence that is at least 70% or 80% identical to the sequence of SEQ ID NO: 605, and the sense strand comprises a sequence that is at least 70% or 80% identical to the sequence of SEQ ID NO: 596; or (i) the antisense strand comprises a sequence that is at least 70% or 80% identical to the sequence of SEQ ID NO: 606, and the sense strand comprises a sequence that is at least 70% or 80% identical to the sequence of SEQ ID NO: 597. In some aspects, the disclosure provides for a double-stranded ribonucleic acid (dsRNA) for inhibiting expression of INHBE, wherein the dsRNA comprises a sense strand and an antisense strand each 15 to 30 nucleotides in length, wherein: the antisense strand comprises a sequence that is at least 70% or 80% identical to the sequence of SEQ ID NO: 616, and the sense strand comprises a sequence that is at least 70% or 80% identical to the sequence of SEQ ID NO: 607; (b) the antisense strand comprises a sequence that is at least 70% or 80% identical to the sequence of SEQ ID NO: 617, and sense strand comprises a sequence that is at least 70% or 80% identical to the sequence of SEQ ID NO: 608; (c) the antisense strand comprises a sequence that is at least 70% or 80% identical to the sequence of SEQ ID NO: 618, and the sense strand comprises a sequence that is at least 70% or 80% identical to the sequence of SEQ ID NO: 609; (d) the antisense strand comprises a sequence that is at least 70% or 80% identical to the sequence of SEQ ID NO: 619, and the sense strand comprises a sequence that is at least 70% or 80% identical to the sequence of SEQ ID NO: 610; (e) the antisense strand comprises a sequence that is at least 70% or 80% identical to the sequence of SEQ ID NO: 620, and the sense strand comprises a sequence that is at least 70% or 80% identical to the sequence of SEQ ID NO: 611; (f) the antisense strand comprises a sequence that is at least 70% or 80% identical to the sequence of SEQ ID NO: 621, and the sense strand comprises at least 15 contiguous nucleotides of a sense strand sequence comprising the sequence of SEQ ID NO: 612; (g) the antisense strand comprises at least 15 contiguous nucleotides of an antisense strand sequence comprising the sequence of SEQ ID NO: 622, and the sense strand comprises a sequence that is at least 70% or 80% identical to the sequence of SEQ ID NO: 613; (h) the antisense strand comprises a sequence that is at least 70% or 80% identical to the sequence of SEQ ID NO: 623, and the sense strand comprises a sequence that is at least 70% or 80% identical to the sequence of SEQ ID NO: 614; or (i) the antisense strand comprises a sequence that is at least 70% or 80% identical to the sequence of SEQ ID NO: 624, and the sense strand comprises a sequence that is at least 70% or 80% identical to the sequence of SEQ ID NO: 615.

In some aspects, the disclosure provides for a double-stranded ribonucleic acid (dsRNA) for inhibiting expression of INHBE comprising a sense strand and an antisense strand each 15 to 30 nucleotides in length, wherein: (a) the antisense strand comprises at least 15 contiguous nucleotides of an antisense strand sequence comprising the sequence of SEQ ID NO: 598, and the sense strand comprises at least 15 contiguous nucleotides of a sense strand sequence comprising the sequence of SEQ ID NO: 589; (b) the antisense strand comprises at least 15 contiguous nucleotides of an antisense strand sequence comprising the sequence of SEQ ID NO: 599, and the sense strand comprises at least 15 contiguous nucleotides of a sense strand sequence comprising the sequence of SEQ ID NO: 590; (c) the antisense strand comprises at least 15 contiguous nucleotides of an antisense strand sequence comprising the sequence of SEQ ID NO: 600, and the sense strand comprises at least 15 contiguous nucleotides of a sense strand sequence comprising the sequence of SEQ ID NO: 591; (d) the antisense strand comprises at least 15 contiguous nucleotides of an antisense strand sequence comprising the sequence of SEQ ID NO: 601, and the sense strand comprises at least 15 contiguous nucleotides of a sense strand sequence comprising the sequence of SEQ ID NO: 592; (e) the antisense strand comprises at least 15 contiguous nucleotides of an antisense strand sequence comprising the sequence of SEQ ID NO: 602, and the sense strand comprises at least 15 contiguous nucleotides of a sense strand sequence comprising the sequence of SEQ ID NO: 593; (f) the antisense strand comprises at least 15 contiguous nucleotides of an antisense strand sequence comprising the sequence of SEQ ID NO: 603, and the sense strand comprises at least 15 contiguous nucleotides of a sense strand sequence comprising the sequence of SEQ ID NO: 594; (g) the antisense strand comprises at least 15 contiguous nucleotides of an antisense strand sequence comprising the sequence of SEQ ID NO: 604, and the sense strand comprises at least 15 contiguous nucleotides of a sense strand sequence comprising the sequence of SEQ ID NO: 595; (h) the antisense strand comprises at least 15 contiguous nucleotides of an antisense strand sequence comprising the sequence of SEQ ID NO: 605, and the sense strand comprises at least 15 contiguous nucleotides of a sense strand sequence comprising the sequence of SEQ ID NO: 596; or (i) the antisense strand comprises at least 15 contiguous nucleotides of an antisense strand sequence comprising the sequence of SEQ ID NO: 606, and the sense strand comprises at least 15 contiguous nucleotides of a sense strand sequence comprising the sequence of SEQ ID NO: 597. In some aspects, the disclosure provides for a double-stranded ribonucleic acid (dsRNA) for inhibiting expression of INHBE, wherein the dsRNA comprises a sense strand and an antisense strand each 15 to 30 nucleotides in length, wherein: the antisense strand comprises at least 15 contiguous nucleotides of an antisense strand sequence comprising the sequence of SEQ ID NO: 616, and the sense strand comprises at least 15 contiguous nucleotides of a sense strand sequence comprising the sequence of SEQ ID NO: 607; (b) the antisense strand comprises at least 15 contiguous nucleotides of an antisense strand sequence comprising the sequence of SEQ ID NO: 617, and the sense strand comprises at least 15 contiguous nucleotides of a sense strand sequence comprising the sequence of SEQ ID NO: 608; (c) the antisense strand comprises at least 15 contiguous nucleotides of an antisense strand sequence comprising the sequence of SEQ ID NO: 618, and the sense strand comprises at least 15 contiguous nucleotides of a sense strand sequence comprising the sequence of SEQ ID NO: 609; (d) the antisense strand comprises at least 15 contiguous nucleotides of an antisense strand sequence comprising the sequence of SEQ ID NO: 619, and the sense strand comprises at least 15 contiguous nucleotides of a sense strand sequence comprising the sequence of SEQ ID NO: 610; (e) the antisense strand comprises at least 15 contiguous nucleotides of an antisense strand sequence comprising the sequence of SEQ ID NO: 620, and the sense strand comprises at least 15 contiguous nucleotides of a sense strand sequence comprising the sequence of SEQ ID NO: 611; (f) the antisense strand comprises at least 15 contiguous nucleotides of an antisense strand sequence comprising the sequence of SEQ ID NO: 621, and the sense strand comprises at least 15 contiguous nucleotides of a sense strand sequence comprising the sequence of SEQ ID NO: 612; (g) the antisense strand comprises at least 15 contiguous nucleotides of an antisense strand sequence comprising the sequence of SEQ ID NO: 622, and the sense strand comprises at least 15 contiguous nucleotides of a sense strand sequence comprising the sequence of SEQ ID NO: 613; (h) the antisense strand comprises at least 15 contiguous nucleotides of an antisense strand sequence comprising the sequence of SEQ ID NO: 623, and the sense strand comprises at least 15 contiguous nucleotides of a sense strand sequence comprising the sequence of SEQ ID NO: 614; or (i) the antisense strand comprises at least 15 contiguous nucleotides of an antisense strand sequence comprising the sequence of SEQ ID NO: 624, and the sense strand comprises at least 15 contiguous nucleotides of a sense strand sequence comprising the sequence of SEQ ID NO: 615.

In some embodiments, the INHBE gene is human INHBE. In some embodiments, the INHBE is human INHBE comprising the sequence shown in SEQ ID NO: 588 (NM_031479.5).

In some embodiments, the sense strand is 70%, 80%, 90%, 95% or more identical to the sense strand sequence comprising the sequence of SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, or SEQ ID NO: 615. In some embodiments, the sense strand comprises at least 16, 17, 18, 19, 20, 21, 22, or 23 contiguous nucleotides of a sense strand sequence comprising the sequence of SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, ID NO: 597, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, or SEQ ID NO: 615. In some embodiments, the sense strand comprises: (a) 20 contiguous nucleotides of a sense strand sequence comprising the sequence of SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, or SEQ ID NO: 615; (b) 21 contiguous nucleotides of a sense strand sequence comprising the sequence of SEQ ID NO: 594, SEQ ID NO: 595, SEQ ID NO: 596, SEQ ID NO: 597, SEQ ID NO: 612, SEQ ID NO: 613, SEQ ID NO: 614, or SEQ ID NO: 615; (c) 22 contiguous nucleotides of a sense strand sequence comprising the sequence of SEQ ID NO: 594, SEQ ID NO: 595, SEQ ID NO: 596, SEQ ID NO: 597, SEQ ID NO: 612, SEQ ID NO: 613, SEQ ID NO: 614, or SEQ ID NO: 615; and/or (d) 23 contiguous nucleotides of a sense strand sequence comprising the sequence of SEQ ID NO: 594 or SEQ ID NO: 612. In some embodiments, the antisense strand comprises at least 16, 17, 18, 19, 20, 21, 22, or 23 contiguous nucleotides of an antisense sense strand sequence comprising the sequence of SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, or SEQ ID NO: 624. In some embodiments, the antisense strand comprises: (a) 21 contiguous nucleotides of an antisense sense strand sequence comprising the sequence of SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, or SEQ ID NO: 624; (b) 22 contiguous nucleotides of an antisense sense strand sequence comprising the sequence of SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, or SEQ ID NO: 624; and/or (c) 23 contiguous nucleotides of an antisense sense strand sequence comprising the sequence of SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, or SEQ ID NO: 624. In some embodiments, the sense strand sequence is selected from a sense strand sequence comprising the sequence of SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, ID NO: 597, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, or SEQ ID NO: 615, and the antisense strand is selected from an antisense strand sequence comprising the sequence of SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, or SEQ ID NO: 624. In some embodiments, the sense strand sequence is selected from a sense strand sequence comprising the sequence of SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, ID NO: 597, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, or SEQ ID NO: 615. In some embodiments, the antisense strand is selected from an antisense strand sequence comprising the sequence of SEQ ID NO: 598, SEQ ID NO: 599, SEQ ID NO: 600, SEQ ID NO: 601, SEQ ID NO: 602, SEQ ID NO: 603, SEQ ID NO: 604, SEQ ID NO: 605, SEQ ID NO: 606, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, or SEQ ID NO: 624. In some embodiments, the antisense strand comprises the sequence of SEQ ID NO: 598 and the sense strand comprises the sequence of SEQ ID NO: 589. In some embodiments, the antisense strand comprises the sequence of SEQ ID NO: 599 and the sense strand comprises the sequence of SEQ ID NO: 590. In some embodiments, the antisense strand comprises the sequence of SEQ ID NO: 600 and the sense strand comprises the sequence of SEQ ID NO: 591. In some embodiments, the antisense strand comprises the sequence of SEQ ID NO: 601 and the sense strand comprises the sequence of SEQ ID NO: 592. In some embodiments, the antisense strand comprises the sequence of SEQ ID NO: 602 and the sense strand comprises the sequence of SEQ ID NO: 593. In some embodiments, the antisense strand comprises the sequence of SEQ ID NO: 603 and the sense strand comprises the sequence of SEQ ID NO: 594. In some embodiments, the antisense strand comprises the sequence of SEQ ID NO: 604 and the sense strand comprises the sequence of SEQ ID NO: 595. In some embodiments, the antisense strand comprises the sequence of SEQ ID NO: 605 and the sense strand comprises the sequence of SEQ ID NO: 596. In some embodiments, the antisense strand comprises the sequence of SEQ ID NO: 606 and the sense strand comprises the sequence of SEQ ID NO: 597. In some embodiments, the antisense strand comprises the sequence of SEQ ID NO: 616 and the sense strand comprises the sequence of SEQ ID NO: 607. In some embodiments, the antisense strand comprises the sequence of SEQ ID NO: 617 and the sense strand comprises the sequence of SEQ ID NO: 608. In some embodiments, the antisense strand comprises the sequence of SEQ ID NO: 618 and the sense strand comprises the sequence of SEQ ID NO: 609. In some embodiments, the antisense strand comprises the sequence of SEQ ID NO: 619 and the sense strand comprises the sequence of SEQ ID NO: 610. In some embodiments, the antisense strand comprises the sequence of SEQ ID NO: 620 and the sense strand comprises the sequence of SEQ ID NO: 611. In some embodiments, the antisense strand comprises the sequence of SEQ ID NO: 621 and the sense strand comprises the sequence of SEQ ID NO: 612. In some embodiments, the antisense strand comprises the sequence of SEQ ID NO: 622 and the sense strand comprises the sequence of SEQ ID NO: 613. In some embodiments, the antisense strand comprises the sequence of SEQ ID NO: 623 and the sense strand comprises the sequence of SEQ ID NO: 614. In some embodiments, the antisense strand comprises the sequence of SEQ ID NO: 624 and the sense strand comprises the sequence of SEQ ID NO: 615. In some embodiments, at least one nucleotide of the dsRNA is a modified nucleotide selected from the group consisting of: a 5′-vinyl phosphonate nucleotide, a 2′-O-methyl modified nucleotide, an inverted deoxyribonucleotide (3′-3′ linked nucleotide or 5′-5′ linked nucleotide), a nucleotide comprising a 5′-phosphorothioate group, a 2′-fluoro modified nucleotide, a nucleotide comprising a modified nucleotide component represented by Formula (I):

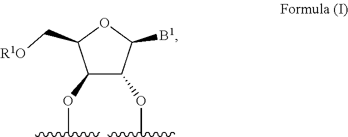

and a nucleotide comprising a modified nucleotide component represented by Formula (II):

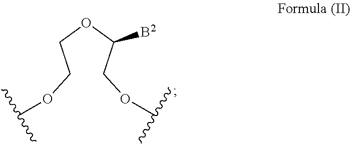

wherein: each of B¹ and B² is a nucleobase; and R¹ is selected from the group consisting of hydrogen and C₁₋₆ alkyl; optionally wherein the antisense strand and the sense strand each comprise at least one modified nucleotide.

In some embodiments, the antisense strand has a 3′ end nucleotide overhang compared to the sense strand. In some embodiments, the 3′ end nucleotide overhang comprises 1, 2, or 3 nucleotides compared to the sense strand. In some embodiments, the antisense and the sense strand are at least 70%, 75%, 80%, 85%, 90%, 95%, or 100% complementary. In some embodiments, the antisense strand and the sense strand are at least 80% complementary. In some embodiments, the antisense strand and the sense strand comprise at least one, at least two, at least three, or at least four mismatched nucleotides. In some embodiments, the antisense strand comprises a nucleotide sequence that is at least about 60%, 65%, 70%, 75%, 80%, 85%, 90%, 95% or 100% identical to a target mRNA corresponding to a fragment of INHBE mRNA. In some embodiments, the antisense strand of the dsRNA comprises at least 80% complementarity to the fragment of the INHBE mRNA. In some embodiments, the antisense strand of the dsRNA comprises one, two, three, or four mismatches to the fragment of the INHBE mRNA.n some embodiments, at least one nucleotide of the dsRNA is a modified nucleotide. In some embodiments, the modified nucleotide is at least one of a modified nucleotide selected from the group consisting of a 2′-O-methyl modified nucleotide, a nucleotide comprising a 5′-phosphorothioate group, a 2′-fluoro modified nucleotide; an inverted abasic nucleotide, a thymidine-glycol nucleic acid (GNA)S-Isomer; an inosine, and inverted deoxyribonucleotide (3′-3′ linked nucleotide or 5′-5′ linked nucleotide), a thymidine-glycol nucleic acid (GNA)S-Isomer, a nucleotide comprising a modified nucleotide component represented by Formula (I):

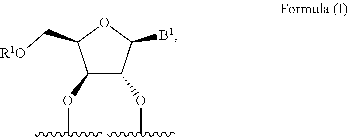

and a nucleotide comprising a modified nucleotide component represented by Formula (II):

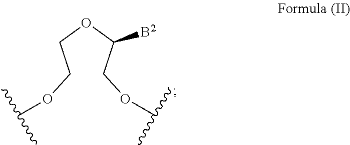

wherein: each of B¹ and B² is a nucleobase; and R¹ is selected from the group consisting of hydrogen and C₁₋₆ alkyl; optionally wherein the antisense strand and the sense strand each comprise at least one modified nucleotide, and a nucleotide comprising a modified nucleotide component represented by Formula (II). In some embodiments, each of B¹ and B² is independently selected from the group consisting of adenine, uracil, thymine, cytosine, guanine, and modified analogs thereof. In some embodiments, each of B¹ and B² is independently selected from adenine, uracil, cytosine, and modified analogs thereof. In some embodiments, R¹ is C₁₋₆ alkyl. In some embodiments, wherein R¹ is —CH₃. In some embodiments, B¹ is uracil. In some embodiments, R¹ is —CH₃ and B¹ is uracil. In some embodiments, B² is adenine. In some embodiments, B² is uracil. In some embodiments, the sense strand comprises an inverted deoxyribonucleotide at the 5′ end; optionally wherein the inverted deoxyribonucleotide is a 5′-5′ linked deoxythymidine. In some embodiments, the sense strand comprises an inverted deoxyribonucleotide at the 3′ end; optionally wherein the inverted deoxyribonucleotide is a 3′-3′ linked deoxythymidine. In some embodiments, the sense strand comprises an inverted deoxyribonucleotide at the 5′ end and an inverted deoxyribonucleotide at the 3′ end; optionally wherein the inverted deoxyribonucleotide at the 5′ end is a 5′-5′ linked deoxythymidine and the inverted deoxyribonucleotide at the 3′ end is a 3′-3′ linked deoxythymidine. In some embodiments, the sense strand comprises a nucleotide comprising the modified nucleotide component represented by Formula (I) at the 3′ end; optionally wherein R¹ is —CH₃ and B¹ is uracil.

In some embodiments, the modified nucleotide is at least one of 5′-vinyl phosphonate nucleotide, a 5′-phosphate or phosphate mimic, a locked nucleic acid (LNA), a 2′-MOE (methoxyethyl)nucleotide, and/or a 2′-arabino fluoro (2′-araF) nucleotide. In some embodiments, the antisense strand comprises a phosphate mimic at the 5′ end; optionally wherein the phosphate mimic is a 5′-E-Vinyl-phosphonate or a 4′-O-phosphonate. In some embodiments, the modified nucleotide is at least one of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and/or a non-natural base comprising nucleotide.

In some embodiments, the antisense strand and/or the sense strand comprises at least one internucleoside linkage selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments, the antisense strand and/or the sense strand comprises at least one nucleotide modified linkage. In some embodiments, all the nucleotide linkages in the antisense strand are modified linkages. In some embodiments, the antisense strand and/or the sense strand comprises at least one a phosphorothioate (PS) bond.

In some embodiments, the dsRNA further comprises a ligand or targeting moiety. In some embodiments, the ligand or targeting moiety is conjugated to the 5′ end, 3′ end or both ends of the dsRNA. In some embodiments, the ligand or targeting moiety is conjugated to the 3′ end of the sense strand of the dsRNA. In some embodiments, the ligand or targeting moiety is conjugated to the 5′ end of the sense strand of the dsRNA. In some embodiments, ligand or targeting moiety is at least one N-Acetyl-Galactosamine (GalNAc). In some embodiments, the ligand or targeting moiety is represented by represented by Formula (I):

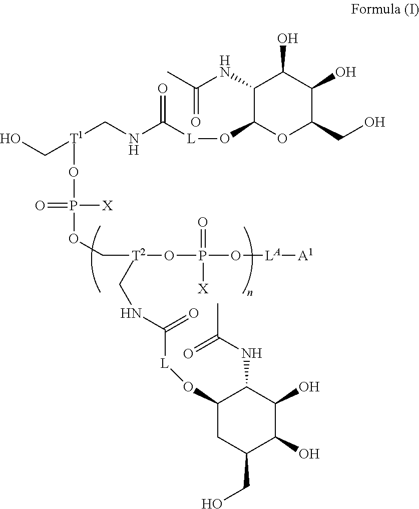

or a pharmaceutically acceptable salt thereof, wherein: A¹ is the point of attachment to the dsRNA; each occurrence of T¹ and T² is independently selected from 5-membered heterocyclyl and alkylene; each occurrence of X is selected from the group consisting of —OH and —SH; and each occurrence of L is a linker; LA is absent or a linker; and n is an integer from 1 to 6. In some embodiments, each occurrence of T¹ and T² is independently selected from 5-membered heterocyclyl having at least one ring oxygen and C₁₋₆ alkylene. In some embodiments, the compound is represented by Formula (I-A):

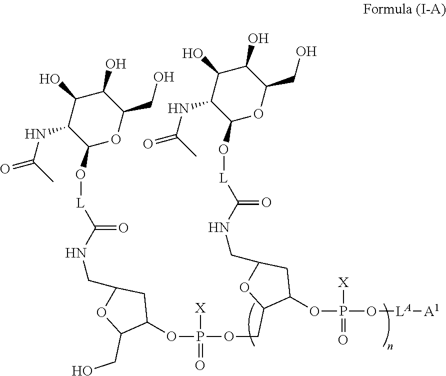

or a pharmaceutically acceptable salt thereof. In some embodiments, the compound is represented by Formula (I-A-I):

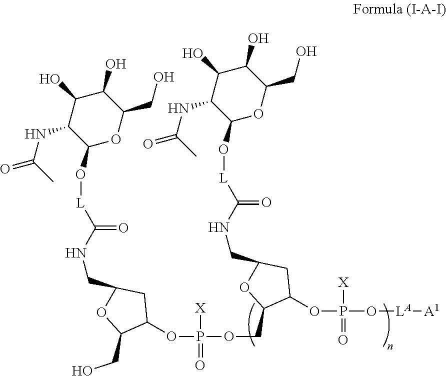

or a pharmaceutically acceptable salt thereof. In some embodiments, the compound is represented by Formula (I-A-II):

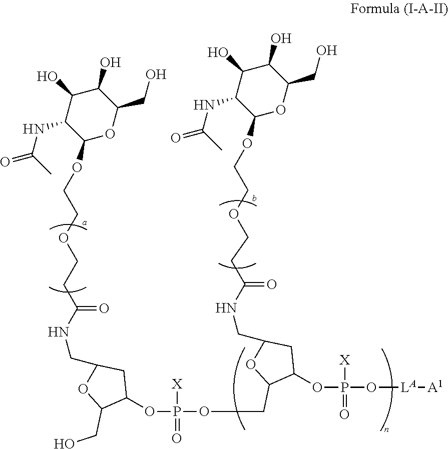

or a pharmaceutically acceptable salt thereof, wherein each occurrence of a and b is an integer from 1-20. In some embodiments, the ligand or targeting moiety is tri-GalNAc6. In some embodiments, the ligand or targeting moiety is L96.

In some embodiments, a cell comprising a dsRNA of the disclosure is provided. In some embodiments, a vector encoding at least one unmodified strand a dsRNA of the disclosure is provided, optionally both strands. of the disclosure is provided a cell comprising the vector is provided.

In some embodiments, a pharmaceutical composition for inhibiting expression of INHBE comprising the dsRNA and a pharmaceutically acceptable carrier, diluent, excipient, or combination thereof of the disclosure is provided.

In some embodiments, a method of inhibiting INHBE expression in a cell is provided, the method comprising (a) contacting the cell with the dsRNA of the disclosure or the pharmaceutical composition of the disclosure; and (b) maintaining the cell produced in step (a) for a time sufficient to obtain degradation of the mRNA transcript of an INHBE gene, thereby inhibiting expression of the INHBE gene in the cell, optionally wherein the method is in vivo. In some embodiments, the INHBE expression is inhibited by at least 30% relative to a control.

In some embodiments, a method of treating a disorder mediated by or associated with INHBE is provided, comprising administering to a subject in need of such treatment a therapeutically effective amount of a dsRNA of the disclosure, or a pharmaceutical composition of the disclosure. In some embodiments, the disorder is a cardiovascular disorder. In some embodiments, the disorder is cardiovascular disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C show bar graphs of the percent (%) inhibition of INHBE mRNA in Huh-7 cells transfected with the indicated GalNAc conjugated, modified siRNA at 10 nM, and 0.1 nM, relative to INHBE mRNA in mock-treated cells. INHBE mRNA level measured by quantitative PCR and normalized to GAPDH.

FIG. 2 shows graphs of exemplary INHBE siRNA compounds in Huh7 cell line in a single dose screen at 100 nM, 33 nM, 11 nM, 3.7 nM, 1.2 nM, 0.412 nM, 0.137 nM, and 0.046 nM of the selected siRNA. INHBE mRNA level was measured by quantitative PCR and normalized to GAPDH relative to mock treated control cells and the average KD and SD was determined.

FIG. 3 shows a bar graph of the percent (%) knockdown of INHBE mRNA in human hepatocytes cells treated with indicated GalNAc conjugated, modified siRNA at 10 nM, and 1 nM relative to INHBE mRNA in PBS treated cells. INHBE mRNA level measured by quantitative PCR and normalized to GAPDH.

FIG. 4 shows a graph of the relative expression of human INHBE mRNA in hydrodynamic injection model with the indicated 13 conjugated, modified siRNA at 1 mg/kg, relative to INHBE expression in PBS treated mice. INHBE mRNA levels were measured by quantitative PCR and normalized to NEO.

FIG. 5 shows a graph of the relative expression of human INHBE mRNA in hydrodynamic injection model with the indicated 12 conjugated, modified siRNA at 1 mg/kg or 1.5 mg/kg, relative to INHBE expression in PBS treated mice. INHBE mRNA levels were measured by quantitative PCR and normalized to NEO.

FIG. 6 shows a bar graph of the relative expression of INHBE mRNA in a non-human primate model treated with siRNA Compounds A and B at 5 mg/kg. INHBE mRNA levels were measured via quantitative PCR using liver biopsy samples, and normalized to INHBE expression level at day −4 for each individual animal.

FIGS. 7A-7C show graphs depicting the agonistic activity of tested compounds in a cell-based hTLR7 reporter assay (FIG. 7A), hTLR8 reporter assay (FIG. 7B), and hTLR9 reporter assay (FIG. 7C). For each graph, the y-axis shows the level of activity as a fold change over unstimulated cells. The x-axis shows the concentration of each compound in nM (log 10 scale).

FIG. 8 shows a volcano plot of differentially expresses genes (DEGs) among different groups in primary human hepatocyte (PHH) cells treated with indicated exemplary siRNA compounds. The x-axis represents the log 2 (FoldChange), while y-axis represents statistical significance for each gene.

FIG. 9 shows graphs depicting the biochemical tests of mice over 7 days after receipt of a single dose of PBS control or siRNA compound 100635, 100642, or 100643. The mean plasma concentration of alanine aminotransferase (ALT), aspartate aminotransferase (AST), triglycerides (TRIG), low-density lipoprotein cholesterol (LDL-C), high-density lipoprotein cholesterol (HDL-C), creatinine (CREZ), cholesterol (CHOL), lactate dehydrogenase (LDH), urine micro total protein (UP), and plasma urea (UREA) is shown.

### DETAILED DESCRIPTION

The details of one or more embodiments of the invention are set forth in the description below. Other features, objects, and advantages of the invention will be apparent from the description, the drawings, and from the claims.

The disclosure provides dsRNA oligonucleotides and methods of using the dsRNA oligonucleotides for inhibiting the expression of a Lipoprotein(A) (INHBE) gene in a cell or a mammal where the dsRNA oligonucleotide targets a INHBE gene. The disclosure also provides compositions and methods for treating pathological conditions and diseases in a mammal caused by the expression of a INHBE gene, e.g., cardiovascular disease. An INHBE dsRNA oligonucleotide directs the sequence-specific degradation of INHBE mRNA.

### I. Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

As used herein, all numerical values or numerical ranges comprise whole integers within or encompassing such ranges and fractions of the values or the integers within or encompassing ranges unless the context clearly indicates otherwise. Thus, for example, reference to a range of 90-100%, comprises 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold comprises 1-, 2-, 3-, 4-, 5-, 6-, 7-, 8-, 9-, 10-, 11-, 12-, 13-, 14-, 15-, 16-, 17-, 18-, 19-, or 20-fold, etc., as well as 1.1-, 1.2-, 1.3-, 1.4-, or 1.5-fold, etc., 2.1-, 2.2-, 2.3-, 2.4-, or 2.5-fold, etc., and so forth.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” or “deoxyribonucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the disclosure by a nucleotide containing, for example, inosine. Sequences comprising such replacement moieties are embodiments of the disclosure.

“INHBE” refers to the Inhibin Subunit Beta E gene. According to the NCBI NLM website, this gene encodes his gene encodes a member of the TGF-beta (transforming growth factor-beta) superfamily of proteins. The encoded preproprotein is proteolytically processed to generate an inhibin beta subunit. Inhibins have been implicated in regulating numerous cellular processes including cell proliferation, apoptosis, immune response and hormone secretion. This gene may be upregulated under conditions of endoplasmic reticulum stress, and this protein may inhibit cellular proliferation and growth in pancreas and liver. A human INHBE mRNA sequence is GenBank accession number NM_031479.5, included herein as SEQ ID NO: 588. A rhesus monkey (Macaca mulatta) INHBE mRNA sequence is GenBank accession number XM_028847001.1.

As used herein, “target sequence” refers to a contiguous portion of the nucleotide sequence of an mRNA molecule formed during the transcription of a INHBE gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person.

For example, a first nucleotide sequence can be described as complementary to a second nucleotide sequence when the two sequences hybridize (e.g., anneal) under stringent hybridization conditions. Hybridization conditions include temperature, ionic strength, pH, and organic solvent concentration for the annealing and/or washing steps. The term stringent hybridization conditions refers to conditions under which a first nucleotide sequence will hybridize preferentially to its target sequence, e.g., a second nucleotide sequence, and to a lesser extent to, or not at all to, other sequences. Stringent hybridization conditions are sequence dependent, and are different under different environmental parameters. Generally, stringent hybridization conditions are selected to be about 5° C. lower than the thermal melting point (Tₘ) for the nucleotide sequence at a defined ionic strength and pH. The Tₘ is the temperature (under defined ionic strength and pH) at which 50% of the first nucleotide sequences hybridize to a perfectly matched target sequence. An extensive guide to the hybridization of nucleic acids is found in, e.g., Tijssen (1993) Laboratory Techniques in Biochemistry and Molecular Biology—Hybridization with Nucleic Acid Probes part I, chap. 2, “Overview of principles of hybridization and the strategy of nucleic acid probe assays,” Elsevier, N.Y. (“Tijssen”).

Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3, or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogsteen base pairing.

The terms “complementary,” “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding INHBE) including a 5′ UTR, an open reading frame (ORF), or a 3′ UTR. For example, a polynucleotide is complementary to at least a part of an INHBE mRNA if the sequence is substantially complementary to a non-interrupted portion of an mRNA encoding INHBE.

In one embodiment, the antisense strand of the dsRNA is sufficiently complementary to a target mRNA so as to cause cleavage of the target mRNA.

The term “double-stranded RNA” or “dsRNA,” as used herein, refers to a complex of ribonucleic acid molecules, having a duplex structure comprising two anti-parallel and substantially complementary, as defined above, nucleic acid strands. In general, the majority of nucleotides of each strand are ribonucleotides, but as described in detail herein, each or both strands can also include at least one non-ribonucleotide, e.g., a deoxyribonucleotide and/or a modified nucleotide. In addition, as used in this specification, “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of this specification and claims.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs. The term “siRNA” is also used herein to refer to a dsRNA as described above.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

“Introducing into a cell,” when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell,” wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection. Further approaches are described herein or known in the art.

The terms “silence,” “inhibit the expression of,” “down-regulate the expression of,” “suppress the expression of” and the like in as far as they refer to a INHBE gene, herein refer to the at least partial suppression of the expression of a INHBE gene, as manifested by a reduction of the amount of mRNA which may be isolated from a first cell or group of cells in which a INHBE gene is transcribed and which has or have been treated such that the expression of a INHBE gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to INHBE gene expression, e.g., the amount of protein encoded by an INHBE gene which is secreted by a cell, the level of plasma lipid levels or the number of cells displaying a certain phenotype. In principle, INHBE gene silencing may be determined in any cell expressing the target, either constitutively or by genomic engineering, and by any appropriate assay. However, when a reference is needed in order to determine whether a given dsRNA inhibits the expression of an INHBE gene by a certain degree and therefore is encompassed by of the disclosure, the assays provided in the Examples below shall serve as such reference.

For example, in certain instances, expression of a INHBE gene is suppressed by at least about 5%, 10%, 15%, 20%, 25%, 30%, 35%, 40%, 45%, or 50% by administration of the double-stranded oligonucleotide of the disclosure. In some embodiments, a INHBE gene is suppressed by at least about 60%, 70%, or 80% by administration of the double-stranded oligonucleotide of the disclosure. In some embodiments, a INHBE gene is suppressed by at least about 85%, 90%, or 95% by administration of the double-stranded of the disclosure.

As used herein, in the context of INHBE expression, the terms “treat,” “treatment,” and the like, refer to relief from or alleviation of pathological processes mediated by INHBE expression. In the context of the present disclosure insofar as it relates to any of the other conditions recited herein below (other than pathological processes mediated by INHBE expression), the terms “treat,” “treatment,” and the like mean to relieve or alleviate at least one symptom associated with such condition, or to slow or reverse the progression of such condition.

As used herein, the phrases “effective amount” refers to an amount that provides a therapeutic benefit in the treatment, prevention, or management of pathological processes mediated by INHBE expression or an overt symptom of pathological processes mediated by INHBE expression. The specific amount that is effective can be readily determined by an ordinary medical practitioner, and may vary depending on factors known in the art, such as, for example, the type of pathological processes mediated by INHBE expression, the patient's history and age, the stage of pathological processes mediated by INHBE expression, and the administration of other anti-pathological processes mediated by INHBE expression agents.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter. For example, a therapeutically effective amount of a dsRNA targeting INHBE can reduce INHBE serum levels by at least 25%.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, “tri-GalNAc6” refers to the structure:

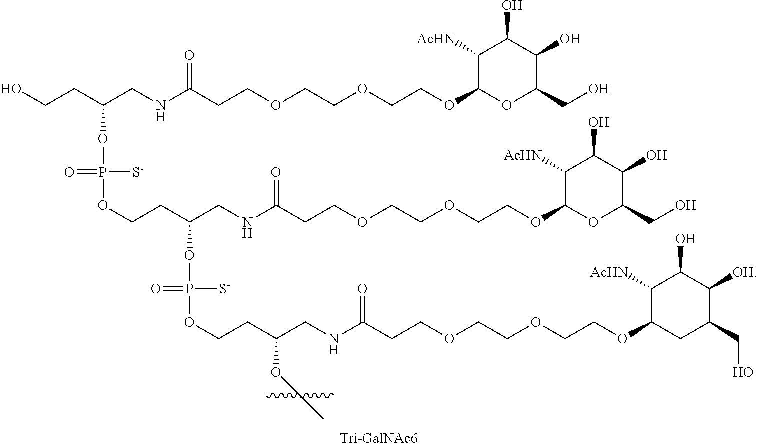 [SMILES: *O[C@H](CCOP(=O)([S-])O[C@H](CCOP(=O)([S-])O[C@H](CCO)CNC(=O)CCOCCOCCO[C@@H]1O[C@H](CO)[C@H](O)[C@H](O)[C@H]1NC(C)=O)CNC(=O)CCOCCOCCO[C@@H]1O[C@H](CO)[C@H](O)[C@H](O)[C@H]1NC(C)=O)CNC(=O)CCOCCOCCO[C@@H]1C[C@H](CO)[C@H](O)[C@H](O)[C@H]1NC(C)=O]

As used herein, “L96 ligand” or “L96” refers to the structure:

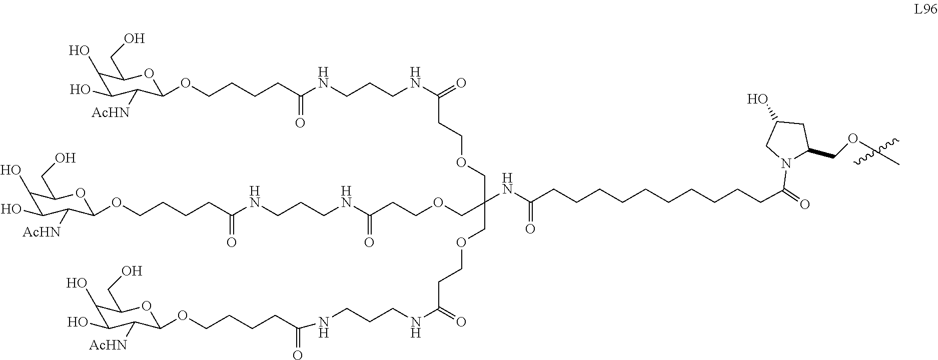

### II. Double-Stranded Ribonucleic Acids (dsRNA)

In one aspect of the disclosure, provided herein are double-stranded ribonucleic acid (dsRNA) molecules for inhibiting the expression of an INHBE gene e.g., in a cell within a subject, such as a mammal (for example a human.) The use of these dsRNA oligonucleotides enables the targeted degradation of mRNAs of the corresponding gene (INHBE gene) in mammals.

In certain embodiments, the dsRNA comprises an antisense strand having a region of complementarity which is complementary to at least a part of an mRNA or an mRNA fragment formed in the expression of a INHBE gene. In some embodiments, the dsRNA comprises at least 70% complementarity to the mRNA or the fragment mRNA of human INHBE mRNA.

In certain embodiments, the dsRNA comprises a sense strand and an antisense strand each 15 to 30 nucleotides in length, wherein the antisense strand comprises at least 15 contiguous nucleotides of an antisense strand sequence shown in Table 1 and Table 2. In certain embodiments, the dsRNA comprises a sense strand and an antisense strand each 15 to 30 nucleotides in length, wherein the antisense strand comprises a sequence that is at least 70% or 80% identical to an antisense strand sequence shown in Table 1 and Table 2. In some embodiments, the dsRNA is Compound 100494, 100506, 100509, 100535, 100557, 100561, 100563, 100563, 100569, 100580, 100589, 100604, 100613, 100625, and 100629. In certain embodiments, the dsRNA comprises a sense strand and an antisense strand each 15 to 30 nucleotides in length, wherein the antisense strand comprises at least 15 contiguous nucleotides of an antisense strand sequence shown in Table 6 and Table 7. In certain embodiments, the dsRNA comprises a sense strand and an antisense strand each 15 to 30 nucleotides in length, wherein the antisense strand comprises a sequence that is at least 70% or 80% identical to an antisense strand sequence shown in Table 6 and Table 7. In some embodiments, the dsRNA is Compound 100635, 100636, 100637, 100638, 100639, 100640, 100641, 100642, 100643, 100644, 100645, 100646, and 100647. In certain embodiments, the dsRNA comprises a sense strand and an antisense strand each 15 to 30 nucleotides in length, wherein the antisense strand comprises at least 15 contiguous nucleotides of an antisense strand sequence shown in Table 9 and Table 10. In certain embodiments, the dsRNA comprises a sense strand and an antisense strand each 15 to 30 nucleotides in length, wherein the antisense strand comprises a sequence that is at least 70% or 80% identical to an antisense strand sequence shown in Table 9 and Table 10. In some embodiments, the dsRNA is Compound 100643, 100647, 100648, 100649, 100650, 100651, 100652, 100653, 100654, 100655, 100656, and 100657. In some embodiments, the INHBE is human INHBE. In some embodiments, the INHBE is human INHBE comprising the sequence shown in SEQ ID NO: 588 (NM_031479.5).

In some embodiments, the sense strand is 70%, 80%, 90%, 95%, or more identical to the sense strands listed in Table 1 and Table 2. In some embodiments, the sense strand is 70%, 80%, 90%, 95%, or more identical to the sense strands listed in Table 6 and Table 7. In some embodiments, the sense strand comprises at least 15 contiguous nucleotides of a sense strand sequence shown in Table 6 and Table 7. In some embodiments, the sense strand is 70%, 80%, 90%, 95%, or more identical to the sense strands listed in Table 9 and Table 10. In some embodiments, the sense strand comprises at least 15 contiguous nucleotides of a sense strand sequence shown in Table 9 and Table 10. In some embodiments, the sense strand comprises at least 15 contiguous nucleotides of a sense strand sequence shown in Table 1 and Table 2. In some embodiments, the sense strand comprises at least 16, 17, 18, 19, 20, or 21 contiguous nucleotides of a sense strand sequence shown in Table 1 and Table 2. In some embodiments, the sense strand comprises 21 contiguous nucleotides of a sense strand sequence shown in Table 1 and Table 2. In some embodiments, the sense strand sequence is selected from a sense strand sequence shown in Table 1 and Table 2. In some embodiments, the sense strand comprises at least 16, 17, 18, 19, 20, 21, 22, or 23 contiguous nucleotides of a sense strand sequence shown in Table 6 and Table 7. In some embodiments, the sense strand comprises 21 contiguous nucleotides of a sense strand sequence shown in Table 6 and Table 7. In some embodiments, the sense strand comprises 22 contiguous nucleotides of a sense strand sequence shown in Table 6 and Table 7. In some embodiments, the sense strand comprises 23 contiguous nucleotides of a sense strand sequence shown in Table 6 and Table 7. In some embodiments, the sense strand sequence is selected from a sense strand sequence shown in Table 6 and Table 7. In some embodiments, the sense strand comprises at least 16, 17, 18, 19, 20, 21, 22, or 23 contiguous nucleotides of a sense strand sequence shown in Table 9 and Table 10. In some embodiments, the sense strand comprises 21 contiguous nucleotides of a sense strand sequence shown in Table 9 and Table 10. In some embodiments, the sense strand comprises 22 contiguous nucleotides of a sense strand sequence shown in Table 9 and Table 10. In some embodiments, the sense strand comprises 23 contiguous nucleotides of a sense strand sequence shown in Table 9 and Table 10. In some embodiments, the sense strand sequence is selected from a sense strand sequence shown in Table 8 and Table 9.

In some embodiments, the antisense strand is 70%, 80%, 90%, 95%, or more identical to the antisense strands listed in Table 1 and Table 2. In some embodiments, the antisense strand comprises at least 16, 17, 18, 19, 20, or 21 contiguous nucleotides of an antisense sense strand sequence shown in Table 1 and Table 2. In some embodiments, the antisense strand comprises 21 contiguous nucleotides of an antisense sense strand sequence shown in Table 1 and Table 2. In some embodiments, the antisense strand is selected from an antisense strand sequence shown in Table 1 and Table 2. In some embodiments, the antisense strand is 70%, 80%, 90%, 95%, or more identical to the antisense strands listed in Table 6 and Table 7. In some embodiments, the antisense strand comprises at least 16, 17, 18, 19, 20, 21, 22, or 23 contiguous nucleotides of an antisense sense strand sequence shown in Table 6 and Table 7. In some embodiments, the antisense strand comprises 21 contiguous nucleotides of an antisense sense strand sequence shown Table 6 and Table 7. In some embodiments, the antisense strand comprises 22 contiguous nucleotides of an antisense sense strand sequence shown Table 6 and Table 7. In some embodiments, the antisense strand comprises 23 contiguous nucleotides of an antisense sense strand sequence shown Table 6 and Table 7. In some embodiments, the antisense strand is selected from an antisense strand sequence shown in Table 6 and Table 7. In some embodiments, the antisense strand is 70%, 80%, 90%, 95%, or more identical to the antisense strands listed in Table 9 and Table 10. In some embodiments, the antisense strand comprises at least 16, 17, 18, 19, 20, 21, 22, or 23 contiguous nucleotides of an antisense sense strand sequence shown in Table 9 and Table 10. In some embodiments, the antisense strand comprises 21 contiguous nucleotides of an antisense sense strand sequence shown Table 9 and Table 10. In some embodiments, the antisense strand comprises 22 contiguous nucleotides of an antisense sense strand sequence shown Table 9 and Table 10. In some embodiments, the antisense strand comprises 23 contiguous nucleotides of an antisense sense strand sequence shown Table 9 and Table 10. In some embodiments, the antisense strand is selected from an antisense strand sequence shown in Table 9 and Table 10.

In some embodiments, the sense strand sequence is selected from a sense strand sequence shown in Table 1 and Table 2, and the antisense strand is selected from an antisense strand sequence shown in Table 1 and Table 2. In some embodiments, the sense strand sequence is selected from a sense strand sequence shown in Table 6 and Table 7, and the antisense strand is selected from an antisense strand sequence shown in Table 6 and Table 7. In some embodiments, the sense strand sequence is selected from a sense strand sequence shown in Table 9 and Table 10, and the antisense strand is selected from an antisense strand sequence shown in Table 9 and Table 10.

In some embodiments, the dsRNA has a mismatch to a fragment of INHBE mRNA. In some embodiments, the dsRNA comprises one or two mismatches to the mRNA or fragment of human INHBE mRNA. In some embodiments, the dsRNA is more than 70% identical to the mRNA or fragment of human INHBE mRNA. In some embodiments, the dsRNA is more than 70%, 75%, 80%, 85%, 90%, or 95% identical to the mRNA or fragment of human INHBE mRNA. In some embodiments, the antisense strand comprises a nucleotide sequence that is at least about 60%, 65%, 70%, 75%, 80%, 85%, 90%, 95%, or 100% identical to a target mRNA corresponding to a fragment of INHBE mRNA. In some embodiments, the antisense strand of the dsRNA comprises at least 80% complementarity to the fragment of the INHBE mRNA. In some embodiments, the mismatch is in the sense strand. In some embodiments, the mismatch is in the antisense strand. In some embodiments, the antisense strand of the dsRNA comprises one, two, three, or four mismatches to the fragment of the INHBE mRNA. In some embodiments, the mismatch is located in the middle of the dsRNA. In some embodiments, the mismatch is in the 5′ or 3′ region of the dsRNA. In some embodiments, the mismatch is no more than 5 nucleotides from the 5′ or 3′ end of the dsRNA.

In some embodiments, at least one strand of the dsRNA comprises a 3′ or 5′ overhang of at least 1 nucleotide. In some embodiments, the overhang is at least 2 or a at least 3 nucleotides. In some embodiments, in the dsRNA at least one strand comprises a 3′ overhang. In some embodiments, in the dsRNA at least one strand comprises a 5′ overhang.

In some embodiments, the antisense strand has a 3′ end nucleotide overhang compared to the sense strand. In some embodiments, the 3′ end nucleotide overhang comprises 1, 2, or 3 nucleotides compared to the sense strand. In some embodiments, the antisense and the sense strand are at least 70%, 75%, 80%, 85%, 90%, 95%, or 100% complementary. In some embodiments, the antisense strand and the sense strand are at least 80% complementary. In some embodiments, the antisense strand and the sense strand comprise at least one, at least two, at least three, or at least four mismatched nucleotides.

The dsRNA can be synthesized by standard methods known in the art as further discussed below, e.g., by use of an automated DNA synthesizer, such as are commercially available from, for example, Biosearch, Applied Biosystems, Inc. The dsRNA includes two RNA strands that are sufficiently complementary to hybridize to form a duplex structure. One strand of the dsRNA (the antisense strand) includes a region of complementarity that is complementary to a target sequence, derived from the sequence of an mRNA formed during the expression of a INHBE gene, the other strand (the sense strand) includes a region that is complementary to the antisense strand, such that the two strands hybridize and form a duplex structure when combined under suitable conditions.

In some embodiments, the duplex structure is between 15 and 30 or between 25 and 30, or between 18 and 25, or between 19 and 24, or between 19 and 21, or 19, 20, or 21 base pairs in length. In one embodiment the duplex is 19 base pairs in length. In another embodiment the duplex is 20 base pairs in length. In another embodiment the duplex is 21 base pairs in length. When two different single stranded RNAs (ssRNA) are used in combination, the duplex lengths can be identical or can differ.

In some embodiments, each strand of the dsRNA of the disclosure is between 15 and 30, or between 18 and 25, or 18, 19, 20, 21, 22, 23, 24, or 25 nucleotides in length. In other embodiments, each is strand is about 25-30 nucleotides in length. In some embodiments, each strand of the duplex is the same length or of different lengths. When two different ssRNAs are used in combination, the lengths of each strand of each ssRNA can be identical or can differ.

In some embodiments, the dsRNA includes dsRNA that is longer than 21-23 nucleotides, e.g., dsRNA that is long enough to be processed by the RNase III enzyme Dicer into 21-23 base pair siRNA which is then incorporated into a RNA-induced silencing complex (RISC). Accordingly, a dsRNA of the disclosure is at least 25, 26, 27, 28, 29, 30, 40, 50, 60, 70, 80, 90, or at least 100 base pairs in length.

Inhibition of the expression of the INHBE gene can be assayed by, for example, a nucleic acid based assay, such as by quantitative PCR, or by a protein-based method, such as by Western blot. Expression of a INHBE gene can be reduced by at least 50% when measured by an assay as described in the Examples below. For example, expression of a INHBE gene in cell culture, such as in Huh-7 cells, can be assayed by measuring INHBE mRNA levels, such as by quantitative PCR assay, or by measuring protein levels, such as by ELISA assay.

In another aspect, the disclosure provides a single-stranded antisense oligonucleotide RNAi. An antisense oligonucleotide is a single-stranded oligonucleotide that is complementary to a sequence within the target mRNA. Antisense oligonucleotides can inhibit translation in a stoichiometric manner by base pairing to the mRNA and physically obstructing the translation machinery, see Dias, N. et al., (2002) Mol. Cancer Ther. 1:347-355. Antisense oligonucleotides can also inhibit target protein expression by binding to the mRNA target and promoting mRNA target destruction via Rnase-H. The single-stranded antisense RNA molecule can be about 13 to about 30 nucleotides in length and have a sequence that is complementary to a target sequence. For example, the single-stranded antisense RNA molecule can comprise a sequence that is at least about 13, 14, 15, 16, 17, 18, 19, 20, or more contiguous nucleotides from one of the antisense sequences in Table 1 and Table 2, Table 6 and Table 7, or Table 9 and Table 10.

### Modifications

In certain embodiments, the dsRNA is chemically modified to enhance stability of the dsRNA. The nucleic acids featured in the disclosure may be synthesized and/or modified by methods well established in the art, such as those described in “Current protocols in nucleic acid chemistry,” Beaucage, S. L. et al. (Eds.), John Wiley & Sons, Inc., New York, NY, USA, which is hereby incorporated herein by reference. Specific examples of dsRNA compounds useful in this disclosure include dsRNAs containing modified backbones or non natural internucleoside linkages. As defined in this specification, dsRNAs having modified backbones include those that retain a phosphorus atom in the backbone and those that do not have a phosphorus atom in the backbone. For the purposes of this specification, and as sometimes referenced in the art, modified dsRNAs that do not have a phosphorus atom in their internucleoside backbone can also be considered to be oligonucleosides. In some embodiments, a modified dsRNA backbone includes at least one of: a 2′-O-methyl modified nucleotide, a nucleotide comprising a 5′-phosphorothioate group, a 2′-fluoro modified nucleotide; an inverted abasic nucleotide, a thymidine-glycol nucleic acid (GNA)S-Isomer; an inosine, and inverted deoxyribonucleotide (3′-3′ linked nucleotide or 5′-5′ linked nucleotide), a thymidine-glycol nucleic acid (GNA)S-Isomer, a nucleotide comprising a modified nucleotide component represented by Formula (I):

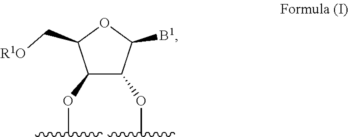

and a nucleotide comprising a modified nucleotide component represented by Formula (II):

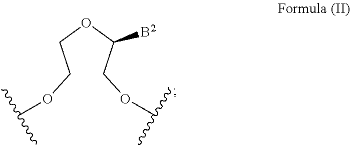

wherein:
each of B¹ and B² is a nucleobase; and
   R¹ is selected from the group consisting of hydrogen and C₁₋₆ alkyl;
optionally wherein the antisense strand and the sense strand each comprise at least one modified nucleotide.

In some embodiments, the nucleotide comprising a modified nucleotide component represented by Formula (I) comprises a nucleobase represented by B¹, wherein B¹ is independently selected from the group consisting of adenine, uracil, thymine, cytosine, guanine, and modified analogs thereof. In some embodiments, the nucleotide comprising a modified nucleotide component represented by Formula (II) comprises a nucleobase represented by B², wherein B² is independently selected from the group consisting of adenine, uracil, thymine, cytosine, guanine, and modified analogs thereof. In some embodiments, each of B¹ and B² is independently selected from adenine, uracil, cytosine, and modified analogs thereof. In some embodiments, R¹ is C₁₋₆ alkyl. In some embodiments, R¹ is —CH₃. In some embodiments, B¹ is uracil. In some embodiments, R¹ is —CH₃ and B¹ is uracil. In some embodiments, B² is adenine. In some embodiments, B² is uracil.

In some embodiments, the sense strand comprises an inverted deoxyribonucleotide at the 3′ end; optionally wherein the inverted deoxyribonucleotide is a 3′-3′ linked deoxythymidine. In some embodiments, the sense strand comprises an inverted deoxyribonucleotide at the 5′ end and an inverted deoxyribonucleotide at the 3′ end; optionally wherein the inverted deoxyribonucleotide at the 5′ end is a 5′-5′ linked deoxythymidine and the inverted deoxyribonucleotide at the 3′ end is a 3′-3′ linked deoxythymidine. In some embodiments, the sense strand comprises a nucleotide comprising the modified nucleotide component represented by Formula (I) at the 3′ end; optionally wherein R¹ is —CH₃ and B¹ is uracil.

In some embodiments, the modification includes one or more phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these) having inverted polarity wherein the adjacent pairs of nucleoside units are linked 3′-5′ to 5′-3′ or 2′-5′ to 5′-2′. Various salts, mixed salts and free acid forms are also included.

In some embodiments, the modified nucleotide includes at least one of: 5′-vinyl phosphonate nucleotide, a 5′-phosphate or phosphate mimic, a locked nucleic acid (LNA), a 2′-MOE (methoxyethyl)nucleotide, and/or a 2′-arabino fluoro (2′-araF) nucleotide. In some embodiments, the modified nucleotide antisense strand comprises a phosphate mimic at the 5′ end; optionally wherein the phosphate mimic is a 5′-E-Vinyl-phosphonate or a 4′-O-phosphonate.

In some embodiments, the modified nucleotide comprises at least one of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and/or a non-natural base comprising nucleotide.

In some embodiments, the antisense strand and/or the sense strand comprises at least one internucleoside linkage selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments, the antisense strand and/or the sense strand comprises at least one nucleotide modified linkage. In some embodiments, all the nucleotide linkages in the antisense strand are modified linkages. In some embodiments, the antisense strand and/or the sense strand comprises at least one a phosphorothioate (PS) bond.

### Conjugates

Another modification of the dsRNAs of the disclosure involves chemically linking to the dsRNA one or more ligand or targeting moieties or conjugates which enhance the activity, cellular distribution or cellular uptake of the dsRNA (e.g., a GalNAc of the present disclosure, e.g., tri-GalNAc6) Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments, the ligand or targeting moiety (e.g., a GalNAc of the present disclosure, e.g., tri-GalNAc6) is conjugated to the 5′ end, 3′ end or both ends of the dsRNA. In some embodiments, the ligand or targeting moiety (e.g., a GalNAc of the present disclosure, e.g., tri-GalNAc6) is conjugated to the 3′ end of the sense strand of the dsRNA. In some embodiments, the ligand or targeting moiety (e.g., a GalNAc of the present disclosure, e.g., tri-GalNAc6) is conjugated to the 3′ end of the antisense strand of the modified dsRNA. In some embodiments, the ligand or targeting moiety (e.g., a GalNAc of the present disclosure, e.g., tri-GalNAc6) is conjugated to the 5′ end of the sense strand of the dsRNA. In some embodiments, the ligand or targeting moiety (e.g., a GalNAc of the present disclosure, e.g., tri-GalNAc6) is conjugated to the 5′ end of the antisense strand of the modified dsRNA. In some embodiments, the ligand or targeting moiety is at least one N-Acetyl-Galactosamine (GalNAc).

In some embodiments, the dsRNA may be modified by a non-ligand group. A number of non-ligand molecules have been conjugated to dsRNAs in order to enhance the activity, cellular distribution or cellular uptake of the dsRNA, and procedures for performing such conjugations are available in the scientific literature. Such non-ligand moieties include lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Typical conjugation protocols involve the synthesis of dsRNAs bearing an aminolinker at one or more positions of the oligonucleotide sequence. The amino group is then reacted with the molecule being conjugated using appropriate coupling or activating reagents. The conjugation reaction may be performed either with the dsRNA still bound to the solid support or following cleavage of the dsRNA in solution phase. The dsRNA conjugate can be purified for example by HPLC methods.

Conjugating a ligand to a dsRNA can enhance its cellular absorption as well as targeting to a particular tissue or uptake by specific types of cells such as liver cells. In certain instances, a hydrophobic ligand is conjugated to the dsRNA to facilitate direct permeation of the cellular membrane and or uptake across the liver cells. Alternatively, the ligand conjugated to the dsRNA is a substrate for receptor-mediated endocytosis. These approaches have been used to facilitate cell permeation of antisense oligonucleotides as well as dsRNA agents. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Li and coworkers report that attachment of folic acid to the 3′-terminus of an oligonucleotide resulted in an 8-fold increase in cellular uptake of the oligonucleotide. Li, S.; Deshmukh, H. M.; Huang, L. Pharm. Res. 1998, 15, 1540. Other ligands that have been conjugated to oligonucleotides include polyethylene glycols, carbohydrate clusters, cross-linking agents, porphyrin conjugates, delivery peptides and lipids such as cholesterol and cholesterylamine. Examples of carbohydrate clusters include Chol-p-(GalNAc)₃ (N-acetyl galactosamine cholesterol) and LCO(GalNAc)₃ (N-acetyl galactosamine-3′-Lithocholic-oleoyl).

### Carbohydrate Conjugates

In some embodiments, a dsRNA oligonucleotide of the disclosure further comprises a carbohydrate. The carbohydrate conjugated dsRNA is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In some embodiments, a carbohydrate conjugate for use in the compositions and methods of the disclosure is a monosaccharide. In some embodiments, the monosaccharide is an N-acetylgalactosamine, of formula I or formula II such as

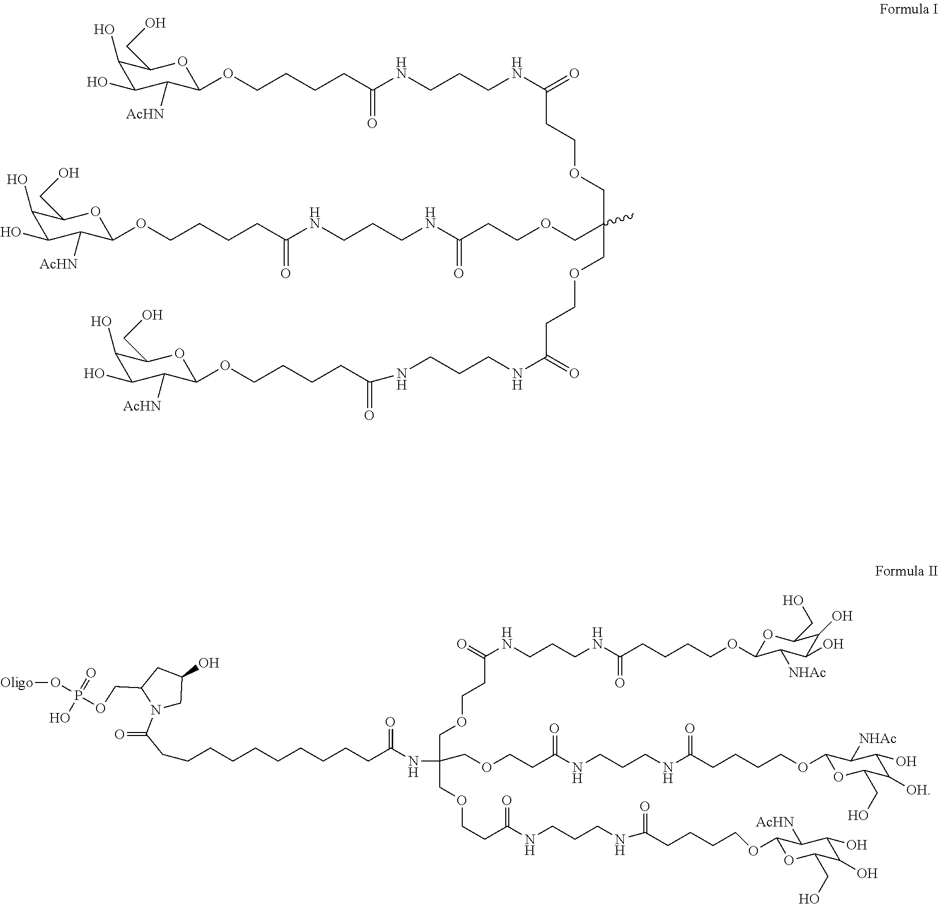

In some embodiments, a carbohydrate is conjugated to the 5′ end, 3′ end or both ends of the modified dsRNA. In some embodiments, the ligand or targeting moiety is conjugated to the 3′ end of the sense strand of the modified dsRNA. In some embodiments, the ligand or targeting moiety is conjugated to the 3′ end of the antisense strand of the modified dsRNA. In some embodiments, the carbohydrate is at least one N-Acetyl-Galactosamine (GalNAc).

### III. Pharmaceutical Compositions

Also disclosed herein are pharmaceutical compositions comprising the dsRNAs targeting INHBE genes of the disclosure.

In certain embodiments, the disclosure provides pharmaceutical compositions containing a dsRNA, as described herein, and a pharmaceutically acceptable carrier. The pharmaceutical composition containing the dsRNA is useful for treating a disease or disorder associated with the expression or activity of a targeting INHBE genes, such as pathological processes mediated by targeting INHBE gene expression. Such pharmaceutical compositions are formulated based on the mode of delivery.

The pharmaceutical compositions featured herein are administered in dosages sufficient to inhibit expression of INHBE genes.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the disclosure can be made using conventional methodologies or on the basis of in vivo testing using an appropriate animal model, as described elsewhere herein.

Advances in mouse genetics have generated a number of mouse models for the study of various human diseases, such as pathological processes mediated by INHBE expression. Such models are used for in vivo testing of dsRNA, as well as for determining a therapeutically effective dose. A suitable mouse model is, for example, a mouse containing a plasmid expressing human INHBE. Another suitable mouse model is a transgenic mouse carrying a transgene that expresses human INHBE.

The data obtained from cell culture assays and animal studies can be used in formulating a range of dosage for use in humans. The dosage of compositions featured in the disclosure lies generally within a range of circulating concentrations that include the ED50 with little or no toxicity. The dosage may vary within this range depending upon the dosage form employed and the route of administration utilized. For any compound used in the methods featured in the disclosure, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The dsRNAs featured in the disclosure can be administered in combination with other known agents effective in treatment of pathological processes mediated by INHBE gene expression. In any event, the administering physician can adjust the amount and timing of dsRNA administration on the basis of results observed using standard measures of efficacy known in the art or described herein.

### Liposomal Formulations

In certain embodiments, pharmaceutical compositions disclosed herein comprise a delivery system. Examples of delivery systems include, but are not limited to, liposomes and emulsions. Certain delivery systems are useful for preparing pharmaceutical compositions including those comprising hydrophobic compounds. In certain embodiments, certain organic solvents such as dimethylsulfoxide are used.

In some embodiments, the dsRNA of the disclosure is introduced into preformed liposomes or lipoplexes made of mixtures of cationic lipids and neutral lipids. In certain methods, dsRNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

### Excipients

In certain embodiments, the pharmaceutical composition comprises an excipient. In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pre-gelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

### Other Components

The compositions of the present disclosure may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

### Administration

Also disclosed herein are methods of administration for the pharmaceutical compositions and formulations which include the dsRNA compositions and pharmaceutical compositions of the disclosure. In some embodiments, the dsRNA composition or pharmaceutical composition of the disclosure is administered in a number of ways depending upon whether local or systemic treatment is desired and upon the area to be treated.

In certain embodiments, administration of the pharmaceutical composition is topical (including buccal and sublingual), pulmonary, e.g., by inhalation or insufflation of powders or aerosols, including by nebulizer; intratracheal, intranasal, epidermal and transdermal, oral or parenteral. In some embodiments, parenteral administration includes intravenous, intraarterial, subcutaneous, intraperitoneal or intramuscular injection or infusion; or intracranial, e.g., intraparenchymal, intrathecal or intraventricular, administration.

Pharmaceutical compositions containing a dsRNA of the disclosure, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, for example, are sterile. Sterilization can be accomplished, for example, by filtration through sterile filtration membranes. Where the composition is lyophilized, filter sterilization can be conducted prior to or following lyophilization and reconstitution.

In certain embodiments, the dsRNA is delivered in a manner to target a particular tissue, for example the liver.

The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound (e.g., dsRNA molecule) which produces a therapeutic effect.

In certain embodiments, a formulation of the present disclosure comprises an excipient selected from the group consisting of cyclodextrins, celluloses, liposomes, micelle forming agents, e.g., bile acids, and polymeric carriers, e.g., polyesters and polyanhydrides; and a compound (e.g., dsRNA molecule) of the present disclosure. In certain embodiments, an aforementioned formulation renders orally bioavailable a compound (e.g., dsRNA molecule) of the present disclosure.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound (e.g., dsRNA molecule) of the present disclosure as an active ingredient. A compound (e.g., dsRNA molecule) of the present disclosure may also be administered as a bolus, electuary or paste.

Liquid dosage forms for oral administration of the compounds (e.g., dsRNA molecules) of the disclosure include pharmaceutically acceptable emulsions, microemulsions, solutions, suspensions, syrups and elixirs.

### IV. Methods for Inhibiting Expression of an INHBE Gene

In one aspect, the disclosure provides a method for inhibiting the expression of an INHBE gene in a cell. The method comprises administering a dsRNA targeting an INHBE gene to a cell, such that expression of the target INHBE gene in the cell is reduced. The disclosure includes methods performed in cells in in vitro or in vivo. In some embodiments, the method is performed in the cell of an animal, e.g., a mouse, a rat, a non-human primate, or a human.

The present disclosure also provides methods of using a dsRNA of the disclosure and/or a composition containing an dsRNA of the present disclosure to reduce and/or inhibit INHBE expression in a cell. The methods include contacting the cell with a dsRNA of the disclosure and maintaining the cell for a time sufficient to obtain degradation of the mRNA transcript of a INHBE gene, thereby inhibiting expression of the INHBE gene in the cell. Reduction in gene expression can be assessed by any methods known in the art. For example, a reduction in the expression of INHBE may be determined by determining the mRNA expression level of INHBE using methods routine to one of ordinary skill in the art, e.g., Northern blotting, qRT-PCR, by determining the protein level of INHBE using methods routine to one of ordinary skill in the art, such as Western blotting, immunological techniques, and/or by determining a biological activity of INHBE, such as affecting one or more molecules associated with the cellular blood clotting mechanism (or in an in vivo setting, blood clotting itself).

In the methods of the disclosure the cell may be contacted in vitro or in vivo, i.e., the cell may be within a subject.

A cell suitable for treatment using the methods of the disclosure may be any cell that expresses a INHBE gene. A cell suitable for use in the methods of the disclosure may be a mammalian cell, e.g., a primate cell (such as a human cell or a non-human primate cell, e.g., a monkey cell or a chimpanzee cell), a non-primate cell (such as a cow cell, a pig cell, a camel cell, a llama cell, a horse cell, a goat cell, a rabbit cell, a sheep cell, a hamster, a guinea pig cell, a cat cell, a dog cell, a rat cell, a mouse cell, a lion cell, a tiger cell, a bear cell, or a buffalo cell), a bird cell (e.g., a duck cell or a goose cell), or a whale cell. In one embodiment, the cell is a human cell, e.g., a human liver cell.

In some embodiments, the INHBE expression is inhibited by at least 30% relative to a control after administration of the dsRNA oligonucleotide of the disclosure.

In some embodiments, a method of treating a disorder mediated by INHBE is provided, comprising administering to a subject in need of such treatment a therapeutically effective amount of an dsRNA oligonucleotide or a pharmaceutical composition of the disclosure.

In some embodiments, the disorder is a cardiovascular disorder. In some embodiments, the disorder is cardiovascular disease.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. In Vitro RNA Interference (RNAi) Screen in Huh-7 Cell Line

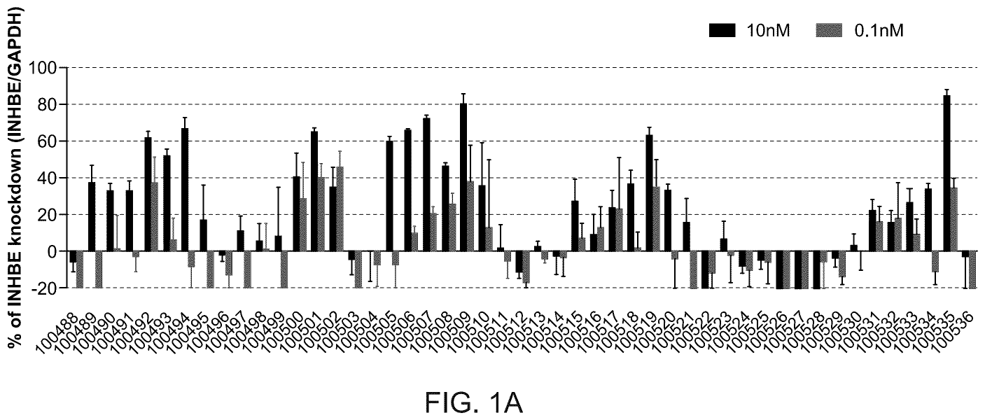

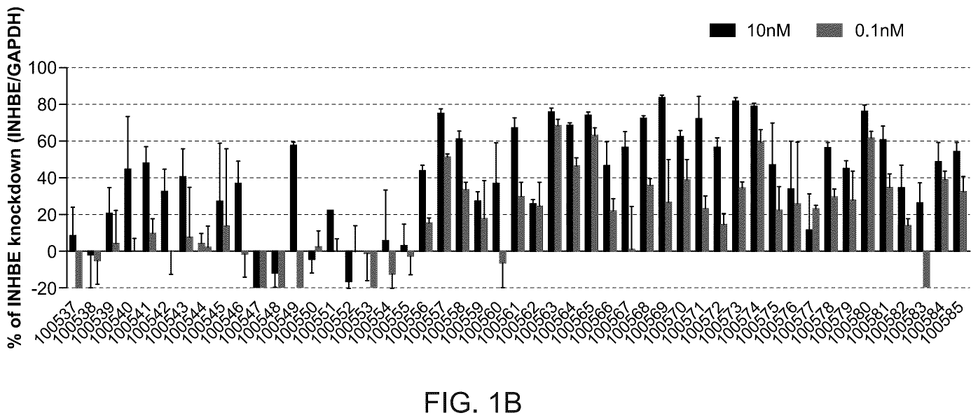

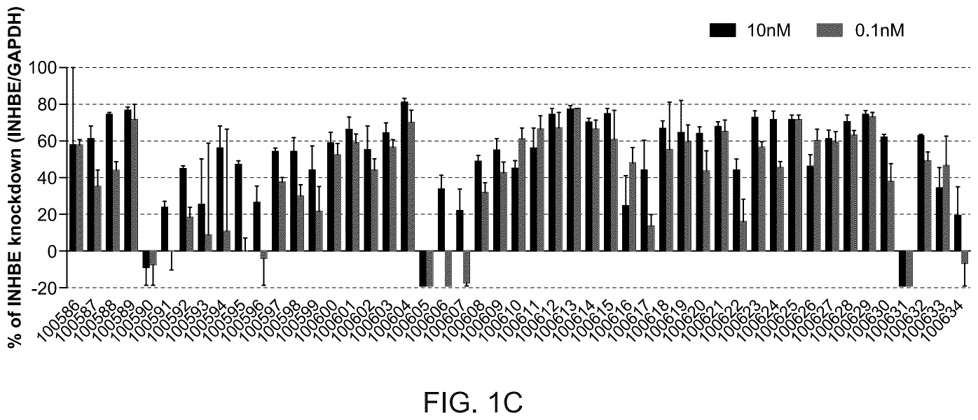

This example describes a screen for siRNA-based inhibition of the INHBE gene in a hepatocyte derived cellular carcinoma cell model (Huh-7). Briefly, Huh-7 cells were transfected were transfected with 147 3′-GalNAc conjugated, modified siRNAs (Duplexes 100488-100634, SEQ ID NOs: 294-440, sense strand; and SEQ ID NOs: 441-587, antisense strand) at 10 nM and 0.1 nM. Sequences of exemplary, unmodified and modified siRNA compounds are shown in Table 1 and Table 2, respectively. Compounds in Table 2 were 3′ GalNac modified. INHBE mRNA level was measured by quantitative PCR and normalized to GAPDH relative to mock treated control cells. Table 3 and FIGS. 1A, B and C show the results of single dose screens at 10 nM and 0.1 nM in Huh7 cells using the selected INHBE siRNAs. The data are presented as percent inhibition of INHBE mRNA in the cells transfected with siRNAs relative to INHBE mRNA in the mock treated control cells.

TABLE 1
[]INHBE siRNA unmodified sequences

| | | | | Position in |
|---|---|---|---|---|
| | SEQ | | SEQ | NM 031479.5 |
| | ID | | ID | (SEQ ID NO: |
| Sense 5′-3′ | NO: | Antisense 5′-3′ | NO: | 588) |
| GGGUCAAGCACAGCUAUCCA | 1 | AUGGAUAGCUGUGCUUGACCCU | 147 | 23 |
| U | | C | | |
| CACAGCUAUCCAUCAGAUGA | 2 | AUCAUCUGAUGGAUAGCUGUGC | 148 | 31 |
| U | | U | | |
| CAGCUAUCCAUCAGAUGAUC | 3 | AGAUCAUCUGAUGGAUAGCUGU | 149 | 33 |
| U | | G | | |
| AGCUAUCCAUCAGAUGAUCU | 4 | UAGAUCAUCUGAUGGAUAGCUG | 150 | 34 |
| A | | U | | |
| CUAUCCAUCAGAUGAUCUAC | 5 | AGUAGAUCAUCUGAUGGAUAGC | 151 | 36 |
| U | | U | | |
| UAUCCAUCAGAUGAUCUACU | 6 | AAGUAGAUCAUCUGAUGGAUAG | 152 | 37 |
| U | | C | | |
| CCAUCAGAUGAUCUACUUUC | 7 | UGAAAGUAGAUCAUCUGAUGGA | 153 | 40 |
| A | | U | | |
| CUACUUUCAGCCUUCCUGAG | 8 | ACUCAGGAAGGCUGAAAGUAGA | 154 | 52 |
| U | | U | | |
| GACAAUAGAAGACAGGUGGC | 9 | AGCCACCUGUCUUCUAUUGUCU | 155 | 77 |
| U | | G | | |
| GCAGUGGUGUCUGCUGUCAC | 10 | AGUGACAGCAGACACCACUGCC | 156 | 123 |
| U | | A | | |
| CUCAUUGGCCCCCAGCAAUC | 11 | UGAUUGCUGGGGGCCAAUGAGG | 157 | 149 |
| A | | G | | |
| CUCCUGUGGGGGCUCCAAAC | 12 | AGUUUGGAGCCCCCACAGGAGG | 158 | 311 |
| U | | G | | |
| CUGGAGCUAGCCAAGCAGCA | 13 | UUGCUGCUUGGCUAGCUCCAGC | 159 | 360 |
| A | | A | | |
| UGGAGCUAGCCAAGCAGCAA | 14 | UUUGCUGCUUGGCUAGCUCCAG | 160 | 361 |
| A | | C | | |
| GGAGCUAGCCAAGCAGCAAA | 15 | AUUUGCUGCUUGGCUAGCUCCA | 161 | 362 |
| U | | G | | |
| GAGCUAGCCAAGCAGCAAAU | 16 | GAUUUGCUGCUUGGCUAGCUCC | 162 | 363 |
| C | | A | | |
| AGCUAGCCAAGCAGCAAAUC | 17 | GGAUUUGCUGCUUGGCUAGCUC | 163 | 364 |
| C | | C | | |
| GCUAGCCAAGCAGCAAAUCC | 18 | AGGAUUUGCUGCUUGGCUAGCU | 164 | 365 |
| U | | C | | |
| UAGCCAAGCAGCAAAUCCUG | 19 | CCAGGAUUUGCUGCUUGGCUAG | 165 | 367 |
| G | | C | | |
| GCCAAGCAGCAAAUCCUGGA | 20 | AUCCAGGAUUUGCUGCUUGGCU | 166 | 369 |
| U | | A | | |
| UGACCAGUCGUCCCAGAAUA | 21 | UUAUUCUGGGACGACUGGUCAG | 167 | 400 |
| A | | G | | |
| ACCAGUCGUCCCAGAAUAAC | 22 | AGUUAUUCUGGGACGACUGGUC | 168 | 402 |
| U | | A | | |
| CGUCCCAGAAUAACUCAUCC | 23 | AGGAUGAGUUAUUCUGGGACGA | 169 | 408 |
| U | | C | | |
| CGCUGACCAGAGCCCUCCGG | 24 | UCCGGAGGGCUCUGGUCAGCGC | 170 | 442 |
| A | | U | | |
| GCUGACCAGAGCCCUCCGGA | 25 | CUCCGGAGGGCUCUGGUCAGCG | 171 | 443 |
| G | | C | | |
| CUGACCAGAGCCCUCCGGAG | 26 | UCUCCGGAGGGCUCUGGUCAGC | 172 | 444 |
| A | | G | | |
| UGACCAGAGCCCUCCGGAGA | 27 | GUCUCCGGAGGGCUCUGGUCAG | 173 | 445 |
| C | | C | | |
| GACCAGAGCCCUCCGGAGAC | 28 | AGUCUCCGGAGGGCUCUGGUCA | 174 | 446 |
| U | | G | | |
| ACCAGAGCCCUCCGGAGACU | 29 | UAGUCUCCGGAGGGCUCUGGUC | 175 | 447 |
| A | | A | | |
| AGGGAAUGGGGAGGAGGUCA | 30 | AUGACCUCCUCCCCAUUCCCUG | 176 | 488 |
| U | | G | | |
| AGGAGGUCAUCAGCUUUGCU | 31 | UAGCAAAGCUGAUGACCUCCUC | 177 | 499 |
| A | | C | | |
| GAGGUCAUCAGCUUUGCUAC | 32 | AGUAGCAAAGCUGAUGACCUCC | 178 | 501 |
| U | | U | | |
| GCUUUGCUACUGUCACAGAC | 33 | AGUCUGUGACAGUAGCAAAGCU | 179 | 511 |
| U | | G | | |
| CGGUCCCACCACCUGUACCA | 34 | AUGGUACAGGUGGUGGGACCGA | 180 | 579 |
| U | | G | | |
| GGUCCCACCACCUGUACCAU | 35 | CAUGGUACAGGUGGUGGGACCG | 181 | 580 |
| G | | A | | |
| GUCCCACCACCUGUACCAUG | 36 | GCAUGGUACAGGUGGUGGGACC | 182 | 581 |
| C | | G | | |
| UCCCACCACCUGUACCAUGC | 37 | GGCAUGGUACAGGUGGUGGGAC | 183 | 582 |
| C | | C | | |
| CCCACCACCUGUACCAUGCC | 38 | GGGCAUGGUACAGGUGGUGGGA | 184 | 583 |
| C | | C | | |
| CCACCACCUGUACCAUGCCC | 39 | CGGGCAUGGUACAGGUGGUGGG | 185 | 584 |
| G | | A | | |
| CACCACCUGUACCAUGCCCG | 40 | GCGGGCAUGGUACAGGUGGUGG | 186 | 585 |
| C | | G | | |
| ACCACCUGUACCAUGCCCGC | 41 | GGCGGGCAUGGUACAGGUGGUG | 187 | 586 |
| C | | G | | |
| CCACCUGUACCAUGCCCGCC | 42 | AGGCGGGCAUGGUACAGGUGGU | 188 | 587 |
| U | | G | | |
| CACCUGUACCAUGCCCGCCU | 43 | CAGGCGGGCAUGGUACAGGUGG | 189 | 588 |
| G | | U | | |
| CCACCCUUCCUGGCACUCUU | 44 | AAAGAGUGCCAGGAAGGGUGGG | 190 | 625 |
| U | | G | | |
| CCCUUCCUGGCACUCUUUGC | 45 | AGCAAAGAGUGCCAGGAAGGGU | 191 | 628 |
| U | | G | | |
| UGGCACUCUUUGCUUGAGGA | 46 | AUCCUCAAGCAAAGAGUGCCAG | 192 | 635 |
| U | | G | | |
| GCACUCUUUGCUUGAGGAUC | 47 | AGAUCCUCAAGCAAAGAGUGCC | 193 | 637 |
| U | | A | | |
| CACUCUUUGCUUGAGGAUCU | 48 | AAGAUCCUCAAGCAAAGAGUGC | 194 | 638 |
| U | | C | | |
| CUAGUGGCUUGAGGGGUGAG | 49 | UCUCACCCCUCAAGCCACUAGA | 195 | 754 |
| A | | G | | |
| GGCUUGAGGGGUGAGAAGUC | 50 | AGACUUCUCACCCCUCAAGCCA | 196 | 759 |
| U | | C | | |
| GAAGUCUGGUGUCCUGAAAC | 51 | AGUUUCAGGACACCAGACUUCU | 197 | 773 |
| U | | C | | |
| UGGUGUCCUGAAACUGCAAC | 52 | AGUUGCAGUUUCAGGACACCAG | 198 | 779 |
| U | | A | | |
| GGUGUCCUGAAACUGCAACU | 53 | UAGUUGCAGUUUCAGGACACCA | 199 | 780 |
| A | | G | | |
| CAGCCCUUCCUAGAGCUUAA | 54 | CUUAAGCUCUAGGAAGGGCUGC | 200 | 876 |
| G | | U | | |
| GCCCUUCCUAGAGCUUAAGA | 55 | AUCUUAAGCUCUAGGAAGGGCU | 201 | 878 |
| U | | G | | |
| GAGCUUAAGAUCCGAGCCAA | 56 | AUUGGCUCGGAUCUUAAGCUCU | 202 | 888 |
| U | | A | | |
| CCAUUACGUAGACUUCCAGG | 57 | UCCUGGAAGUCUACGUAAUGGU | 203 | 983 |
| A | | C | | |
| CCCGAGGGGUACCAGCUGAA | 58 | AUUCAGCUGGUACCCCUCGGGC | 204 | 1032 |
| U | | U | | |
| CGAGGGGUACCAGCUGAAUU | 59 | UAAUUCAGCUGGUACCCCUCGG | 205 | 1034 |
| A | | G | | |
| GGUACCAGCUGAAUUACUGC | 60 | UGCAGUAAUUCAGCUGGUACCC | 206 | 1039 |
| A | | C | | |
| GUACCAGCUGAAUUACUGCA | 61 | CUGCAGUAAUUCAGCUGGUACC | 207 | 1040 |
| G | | C | | |
| UACCAGCUGAAUUACUGCAG | 62 | ACUGCAGUAAUUCAGCUGGUAC | 208 | 1041 |
| U | | C | | |
| ACCAGCUGAAUUACUGCAGU | 63 | CACUGCAGUAAUUCAGCUGGUA | 209 | 1042 |
| G | | C | | |
| CCAGCUGAAUUACUGCAGUG | 64 | CCACUGCAGUAAUUCAGCUGGU | 210 | 1043 |
| G | | A | | |
| AGCUGAAUUACUGCAGUGGG | 65 | GCCCACUGCAGUAAUUCAGCUG | 211 | 1045 |
| C | | G | | |
| GCUGAAUUACUGCAGUGGGC | 66 | UGCCCACUGCAGUAAUUCAGCU | 212 | 1046 |
| A | | G | | |
| CUGAAUUACUGCAGUGGGCA | 67 | CUGCCCACUGCAGUAAUUCAGC | 213 | 1047 |
| G | | U | | |
| AUUACUGCAGUGGGCAGUGC | 68 | GGCACUGCCCACUGCAGUAAUU | 214 | 1051 |
| C | | C | | |
| GGCAUUGCUGCCUCUUUCCA | 69 | AUGGAAAGAGGCAGCAAUGCCU | 215 | 1095 |
| U | | G | | |
| GCAUUGCUGCCUCUUUCCAU | 70 | AAUGGAAAGAGGCAGCAAUGCC | 216 | 1096 |
| U | | U | | |
| CUCUUUCCAUUCUGCCGUCU | 71 | AAGACGGCAGAAUGGAAAGAGG | 217 | 1106 |
| U | | C | | |
| UCAGCCUCCUCAAAGCCAAC | 72 | UGUUGGCUUUGAGGAGGCUGAA | 218 | 1126 |
| A | | G | | |
| CAGCCUCCUCAAAGCCAACA | 73 | UUGUUGGCUUUGAGGAGGCUGA | 219 | 1127 |
| A | | A | | |
| UCCUCAAAGCCAACAAUCCU | 74 | AAGGAUUGUUGGCUUUGAGGAG | 220 | 1132 |
| U | | G | | |
| UCUCUCCUCUACCUGGAUCA | 75 | AUGAUCCAGGUAGAGGAGAGAG | 221 | 1200 |
| U | | A | | |
| UCUCCUCUACCUGGAUCAUA | 76 | UUAUGAUCCAGGUAGAGGAGAG | 222 | 1202 |
| A | | A | | |
| CUCCUCUACCUGGAUCAUAA | 77 | AUUAUGAUCCAGGUAGAGGAGA | 223 | 1203 |
| U | | G | | |
| UACCUGGAUCAUAAUGGCAA | 78 | AUUGCCAUUAUGAUCCAGGUAG | 224 | 1209 |
| U | | A | | |
| CCUGGAUCAUAAUGGCAAUG | 79 | ACAUUGCCAUUAUGAUCCAGGU | 225 | 1211 |
| U | | A | | |
| AUCAUAAUGGCAAUGUGGUC | 80 | UGACCACAUUGCCAUUAUGAUC | 226 | 1216 |
| A | | C | | |
| UCAUAAUGGCAAUGUGGUCA | 81 | UUGACCACAUUGCCAUUAUGAU | 227 | 1217 |
| A | | C | | |
| CAUAAUGGCAAUGUGGUCAA | 82 | CUUGACCACAUUGCCAUUAUGA | 228 | 1218 |
| G | | U | | |
| AUAAUGGCAAUGUGGUCAAG | 83 | UCUUGACCACAUUGCCAUUAUG | 229 | 1219 |
| A | | A | | |
| UAAUGGCAAUGUGGUCAAGA | 84 | GUCUUGACCACAUUGCCAUUAU | 230 | 1220 |
| C | | G | | |
| AAUGGCAAUGUGGUCAAGAC | 85 | CGUCUUGACCACAUUGCCAUUA | 231 | 1221 |
| G | | U | | |
| CAAUGUGGUCAAGACGGAUG | 86 | ACAUCCGUCUUGACCACAUUGC | 232 | 1226 |
| U | | C | | |
| CAAGACGGAUGUGCCAGAUA | 87 | AUAUCUGGCACAUCCGUCUUGA | 233 | 1235 |
| U | | C | | |
| GAGGCCUGUGGCUGCAGCUA | 88 | CUAGCUGCAGCCACAGGCCUCC | 234 | 1263 |
| G | | A | | |
| AGGCCUGUGGCUGCAGCUAG | 89 | GCUAGCUGCAGCCACAGGCCUC | 235 | 1264 |
| C | | C | | |
| GGCCUGUGGCUGCAGCUAGC | 90 | UGCUAGCUGCAGCCACAGGCCU | 236 | 1265 |
| A | | C | | |
| GCCUGUGGCUGCAGCUAGCA | 91 | UUGCUAGCUGCAGCCACAGGCC | 237 | 1266 |
| A | | U | | |
| CCUGUGGCUGCAGCUAGCAA | 92 | CUUGCUAGCUGCAGCCACAGGC | 238 | 1267 |
| G | | C | | |
| GCUUUGGAGUGAAGAGACCA | 93 | UUGGUCUCUUCACUCCAAAGCC | 239 | 1298 |
| A | | C | | |
| CAACCACCUGGCAAUAUGAC | 94 | AGUCAUAUUGCCAGGUGGUUGU | 240 | 1389 |
| U | | U | | |
| ACCACCUGGCAAUAUGACUC | 95 | UGAGUCAUAUUGCCAGGUGGUU | 241 | 1391 |
| A | | G | | |
| CACCUGGCAAUAUGACUCAC | 96 | AGUGAGUCAUAUUGCCAGGUGG | 242 | 1393 |
| U | | U | | |
| GGACCCAAAUGGGCACUUUC | 97 | AGAAAGUGCCCAUUUGGGUCCC | 243 | 1425 |
| U | | A | | |
| CCCAAAUGGGCACUUUCUUG | 98 | ACAAGAAAGUGCCCAUUUGGGU | 244 | 1428 |
| U | | C | | |
| CAAAUGGGCACUUUCUUGUC | 99 | AGACAAGAAAGUGCCCAUUUGG | 245 | 1430 |
| U | | G | | |
| GGCACUUUCUUGUCUGAGAC | 100 | AGUCUCAGACAAGAAAGUGCCC | 246 | 1436 |
| U | | A | | |
| CACUUUCUUGUCUGAGACUC | 101 | AGAGUCUCAGACAAGAAAGUGC | 247 | 1438 |
| U | | C | | |
| CUUGUCUGAGACUCUGGCUU | 102 | UAAGCCAGAGUCUCAGACAAGA | 248 | 1444 |
| A | | A | | |
| AGGGAAGGCAGAGAAAAAUU | 103 | UAAUUUUUCUCUGCCUUCCCUC | 249 | 1586 |
| A | | C | | |
| GGGAAGGCAGAGAAAAAUUA | 104 | GUAAUUUUUCUCUGCCUUCCCU | 250 | 1587 |
| C | | C | | |
| AGCCUCUCCCAAGAUGAGAA | 105 | UUUCUCAUCUUGGGAGAGGCUA | 251 | 1610 |
| A | | A | | |
| CCUCUCCCAAGAUGAGAAAG | 106 | ACUUUCUCAUCUUGGGAGAGGC | 252 | 1612 |
| U | | U | | |
| CUCCCAAGAUGAGAAAGUCC | 107 | AGGACUUUCUCAUCUUGGGAGA | 253 | 1615 |
| U | | G | | |
| GGGGAGGAGGAAGCAGAUAG | 108 | UCUAUCUGCUUCCUCCUCCCCU | 254 | 1643 |
| A | | C | | |
| GGGAGGAGGAAGCAGAUAGA | 109 | AUCUAUCUGCUUCCUCCUCCCC | 255 | 1644 |
| U | | U | | |
| CAGAAACAGGAGUCAGGAAA | 110 | UUUUCCUGACUCCUGUUUCUGG | 256 | 1786 |
| A | | G | | |
| GCACUAAGCCUAAGAAGUUC | 111 | GGAACUUCUUAGGCUUAGUGCC | 257 | 1811 |
| C | | U | | |
| CCCACUGGGAGACAAGCAUU | 112 | AAAUGCUUGUCUCCCAGUGGGU | 258 | 1855 |
| U | | C | | |
| CCACUGGGAGACAAGCAUUU | 113 | UAAAUGCUUGUCUCCCAGUGGG | 259 | 1856 |
| A | | U | | |
| ACUGGGAGACAAGCAUUUAU | 114 | UAUAAAUGCUUGUCUCCCAGUG | 260 | 1858 |
| A | | G | | |
| UGGGAGACAAGCAUUUAUAC | 115 | AGUAUAAAUGCUUGUCUCCCAG | 261 | 1860 |
| U | | U | | |
| GGGAGACAAGCAUUUAUACU | 116 | AAGUAUAAAUGCUUGUCUCCCA | 262 | 1861 |
| U | | G | | |
| GACAAGCAUUUAUACUUUCU | 117 | AAGAAAGUAUAAAUGCUUGUCU | 263 | 1865 |
| U | | C | | |
| GAGCCACCGCGCCUGGCUUA | 118 | AUAAGCCAGGCGCGGUGGCUCA | 264 | 2153 |
| U | | C | | |
| CCACCGCGCCUGGCUUAUAC | 119 | AGUAUAAGCCAGGCGCGGUGGC | 265 | 2156 |
| U | | U | | |
| ACCGCGCCUGGCUUAUACUU | 120 | AAAGUAUAAGCCAGGCGCGGUG | 266 | 2158 |
| U | | G | | |
| CGCGCCUGGCUUAUACUUUC | 121 | AGAAAGUAUAAGCCAGGCGCGG | 267 | 2160 |
| U | | U | | |
| GCGCCUGGCUUAUACUUUCU | 122 | AAGAAAGUAUAAGCCAGGCGCG | 268 | 2161 |
| U | | G | | |
| CGCCUGGCUUAUACUUUCUU | 123 | UAAGAAAGUAUAAGCCAGGCGC | 269 | 2162 |
| A | | G | | |
| GCCUGGCUUAUACUUUCUUA | 124 | UUAAGAAAGUAUAAGCCAGGCG | 270 | 2163 |
| A | | C | | |
| CCUGGCUUAUACUUUCUUAA | 125 | AUUAAGAAAGUAUAAGCCAGGC | 271 | 2164 |
| U | | G | | |
| UGGCUUAUACUUUCUUAAUA | 126 | UUAUUAAGAAAGUAUAAGCCAG | 272 | 2166 |
| A | | G | | |
| GGCUUAUACUUUCUUAAUAA | 127 | UUUAUUAAGAAAGUAUAAGCCA | 273 | 2167 |
| A | | G | | |
| CUUAUACUUUCUUAAUAAAA | 128 | UUUUUAUUAAGAAAGUAUAAGC | 274 | 2169 |
| A | | C | | |
| AGGGGUGUCCACAAAGUCAA | 129 | UUUGACUUUGUGGACACCCCUG | 275 | 2296 |
| A | | A | | |
| GGGGUGUCCACAAAGUCAAA | 130 | CUUUGACUUUGUGGACACCCCU | 276 | 2297 |
| G | | G | | |
| UCAUAAUAAUACUAACAUGU | 131 | AACAUGUUAGUAUUAUUAUGAA | 277 | 2324 |
| U | | A | | |
| ACUAACAUGUUAUUUGCCUU | 132 | AAAGGCAAAUAACAUGUUAGUA | 278 | 2334 |
| U | | U | | |
| GUUAUUUGCCUUUUGAAUUC | 133 | AGAAUUCAAAAGGCAAAUAACA | 279 | 2342 |
| U | | U | | |
| UGCCUUUUGAAUUCUCAUUA | 134 | AUAAUGAGAAUUCAAAAGGCAA | 280 | 2348 |
| U | | A | | |
| GCCUUUUGAAUUCUCAUUAU | 135 | GAUAAUGAGAAUUCAAAAGGCA | 281 | 2349 |
| C | | A | | |
| CCUUUUGAAUUCUCAUUAUC | 136 | AGAUAAUGAGAAUUCAAAAGGC | 282 | 2350 |
| U | | A | | |
| CUUUUGAAUUCUCAUUAUCU | 137 | AAGAUAAUGAGAAUUCAAAAGG | 283 | 2351 |
| U | | C | | |
| UGAAUUCUCAUUAUCUUAAA | 138 | UUUUAAGAUAAUGAGAAUUCAA | 284 | 2355 |
| A | | A | | |
| GAAUUCUCAUUAUCUUAAAA | 139 | AUUUUAAGAUAAUGAGAAUUCA | 285 | 2356 |
| U | | A | | |
| CCGUGUGACAUGUGAUUACA | 140 | AUGUAAUCACAUGUCACACGGC | 286 | 2400 |
| U | | C | | |
| UGUGACAUGUGAUUACAUCA | 141 | AUGAUGUAAUCACAUGUCACAC | 287 | 2403 |
| U | | G | | |
| UGACAUGUGAUUACAUCAUC | 142 | AGAUGAUGUAAUCACAUGUCAC | 288 | 2405 |
| U | | A | | |
| GACAUGUGAUUACAUCAUCU | 143 | AAGAUGAUGUAAUCACAUGUCA | 289 | 2406 |
| U | | C | | |
| UCUUUCUGACAUCAUUGUUA | 144 | UUAACAAUGAUGUCAGAAAGAU | 290 | 2423 |
| A | | G | | |
| CUUUCUGACAUCAUUGUUAA | 145 | AUUAACAAUGAUGUCAGAAAGA | 291 | 2424 |
| U | | U | | |
| GACAUCAUUGUUAAUGGAAU | 146 | CAUUCCAUUAACAAUGAUGUCA | 292 | 2430 |
| G | | G | | |
| GUUAAUGGAAUGUGUGCUUG | 147 | ACAAGCACACAUUCCAUUAACA | 293 | 2439 |
| U | | A | | |

TABLE 2
[]GalNac modified sense strand and antisense strand sequences conjugated to
3′-GalNAc targeting INHBE mRNA.

| | | SEQ | | SEQ |
|---|---|---|---|---|
| Duplex | | ID | | ID |
| name | Sense 5′-3′ (modified) | NO: | Antisense 5′-3′ (modified) | NO: |
| 100488 | g*g*gucaAgCACagcuauccau | 294 | a*U*ggaUaGCugugCuUgaccc*u*c | 441 |
| 100489 | c*a*cagcUaUCCaucagaugau | 295 | a*U*cauCuGAuggaUaGcugug*c*u | 442 |
| 100490 | c*a*gcuaUcCAUcagaugaucu | 296 | a*G*aucAuCUgaugGaUagcug*u*g | 443 |
| 100491 | a*g*cuauCcAUCagaugaucua | 297 | u*A*gauCaUCugauGgAuagcu*g*u | 444 |
| 100492 | c*u*auccAuCAGaugaucuacu | 298 | a*G*uagAuCAucugAuGgauag*c*u | 445 |
| 100493 | u*a*uccaUcAGAugaucuacuu | 299 | a*A*guaGaUCaucuGaUggaua*g*c | 446 |
| 100494 | c*c*aucaGaUGAucuacuuuca | 300 | u*G*aaaGuAGaucaUcUgaugg*a*u | 447 |
| 100495 | c*u*acuuUcAGCcuuccugagu | 301 | a*C*ucaGgAAggcuGaAaguag*a*u | 448 |
| 100496 | g*a*caauAgAAGacagguggcu | 302 | a*G*ccaCcUGucuuCuAuuguc*u*g | 449 |
| 100497 | g*c*agugGuGUCugcugucacu | 303 | a*G*ugaCaGCagacAcCacugc*c*a | 450 |
| 100498 | c*u*cauuGgCCCccagcaauca | 304 | u*G*auuGcUGggggCcAaugag*g*g | 451 |
| 100499 | c*u*ccugUgGGGgcuccaaacu | 305 | a*G*uuuGgAGccccCaCaggag*g*g | 452 |
| 100500 | c*u*ggagCuAGCcaagcagcaa | 306 | u*U*gcuGcUUggcuAgCuccag*c*a | 453 |
| 100501 | u*g*gagcUaGCCaagcagcaaa | 307 | u*U*ugcUgCUuggcUaGcucca*g*c | 454 |
| 100502 | g*g*agcuAgCCAagcagcaaau | 308 | a*U*uugCuGCuuggCuAgcucc*a*g | 455 |
| 100503 | g*a*gcuaGcCAAgcagcaaauc | 309 | g*A*uuuGcUGcuugGcUagcuc*c*a | 456 |
| 100504 | a*g*cuagCcAAGcagcaaaucc | 310 | g*G*auuUgCUgcuuGgCuagcu*c*c | 457 |
| 100505 | g*c*uagcCaAGCagcaaauccu | 311 | a*G*gauUuGCugcuUgGcuagc*u*c | 458 |
| 100506 | u*a*gccaAgCAGcaaauccugg | 312 | c*C*aggAuUUgcugCuUggcua*g*c | 459 |
| 100507 | g*c*caagCaGCAaauccuggau | 313 | a*U*ccaGgAUuugcUgCuuggc*u*a | 460 |
| 100508 | u*g*accaGuCGUcccagaauaa | 314 | u*U*auuCuGGgacgAcUgguca*g*g | 461 |
| 100509 | a*c*caguCgUCCcagaauaacu | 315 | a*G*uuaUuCUgggaCgAcuggu*c*a | 462 |
| 100510 | c*g*ucccAgAAUaacucauccu | 316 | a*G*gauGaGUuauuCuGggacg*a*c | 463 |
| 100511 | c*g*cugaCcAGAgcccuccgga | 317 | u*C*cggAgGGcucuGgUcagcg*c*u | 464 |
| 100512 | g*c*ugacCaGAGcccuccggag | 318 | C*U*ccgGaGGgcucUgGucagc*g*c | 465 |
| 100513 | c*u*gaccAgAGCccuccggaga | 319 | u*C*uccGgAGggcuCuGgucag*c*g | 466 |
| 100514 | u*g*accaGaGCCcuccggagac | 320 | g*U*cucCgGAgggcUcUgguca*g*c | 467 |
| 100515 | g*a*ccagAgCCCuccggagacu | 321 | a*G*ucuCcGGagggCuCugguc*a*g | 468 |
| 100516 | a*c*cagaGcCCUccggagacua | 322 | u*A*gucUcCGgaggGcUcuggu*c*a | 469 |
| 100517 | a*g*ggaaUgGGGaggaggucau | 323 | a*U*gacCuCCucccCaUucccu*g*g | 470 |
| 100518 | a*g*gaggUcAUCagcuuugcua | 324 | u*A*gcaAaGCugauGaCcuccu*c*c | 471 |
| 100519 | g*a*ggucAuCAGcuuugcuacu | 325 | a*G*uagCaAAgcugAuGaccuc*c*u | 472 |
| 100520 | g*c*uuugCuACUgucacagacu | 326 | a*G*ucuGuGAcaguAgCaaagc*u*g | 473 |
| 100521 | c*g*guccCaCCAccuguaccau | 327 | a*U*gguAcAGguggUgGgaccg*a*g | 474 |
| 100522 | g*g*ucccAcCACcuguaccaug | 328 | c*A*uggUaCAggugGuGggacc*g*a | 475 |
| 100523 | g*u*cccaCcACCuguaccaugc | 329 | g*C*augGuACagguGgUgggac*c*g | 476 |
| 100524 | u*c*ccacCaCCUguaccaugcc | 330 | g*G*cauGgUAcaggUgGuggga*c*c | 477 |
| 100525 | c*c*caccAcCUGuaccaugccc | 331 | g*G*gcaUgGUacagGuGguggg*a*c | 478 |
| 100526 | C*c*accaCcUGUaccaugcccg | 332 | c*G*ggcAuGGuacaGgUggugg*g*a | 479 |
| 100527 | c*a*ccacCuGUAccaugcccgc | 333 | g*C*gggCaUGguacAgGuggug*g*g | 480 |
| 100528 | a*c*caccUgUACcaugcccgcc | 334 | g*G*cggGcAUgguaCaGguggu*g*g | 481 |
| 100529 | c*c*accuGuACCaugcccgccu | 335 | a*G*gcgGgCAugguAcAggugg*u*g | 482 |
| 100530 | c*a*ccugUaCCAugcccgccug | 336 | c*A*ggcGgGCauggUaCaggug*g*u | 483 |
| 100531 | c*c*acccUuCCUggcacucuuu | 337 | a*A*agaGuGCcaggAaGggugg*g*g | 484 |
| 100532 | c*c*cuucCuGGCacucuuugcu | 338 | a*G*caaAgAGugccAgGaaggg*u*g | 485 |
| 100533 | u*g*gcacUcUUUgcuugaggau | 339 | a*U*ccuCaAGcaaaGaGugcca*g*g | 486 |
| 100534 | g*c*acucUuUGCuugaggaucu | 340 | a*G*aucCuCAagcaAaGagugc*c*a | 487 |
| 100535 | c*a*cucuUuGCUugaggaucuu | 341 | a*A*gauCcUCaagcAaAgagug*c*c | 488 |
| 100536 | c*u*agugGcUUGaggggugaga | 342 | u*C*ucaCcCCucaaGcCacuag*a*g | 489 |
| 100537 | g*g*cuugAgGGGugagaagucu | 343 | a*G*acuUcUCacccCuCaagcc*a*c | 490 |
| 100538 | g*a*agucUgGUGuccugaaacu | 344 | a*G*uuuCaGGacacCaGacuuc*u*c | 491 |
| 100539 | u*g*guguCcUGAaacugcaacu | 345 | a*G*uugCaGUuucaGgAcacca*g*a | 492 |
| 100540 | g*g*ugucCuGAAacugcaacua | 346 | u*A*guuGcAGuuucAgGacacc*a*g | 493 |
| 100541 | c*a*gcccUuCCUagagcuuaag | 347 | c*U*uaaGcUCuaggAaGggcug*c*u | 494 |
| 100542 | g*c*ccuuCcUAGagcuuaagau | 348 | a*U*cuuAaGCucuaGgAagggc*u*g | 495 |
| 100543 | g*a*gcuuAaGAUccgagccaau | 349 | a*U*uggCuCGgaucUuAagcuc*u*a | 496 |
| 100544 | c*c*auuaCgUAGacuuccagga | 350 | u*C*cugGaAGucuaCgUaaugg*u*c | 497 |
| 100545 | c*c*cgagGgGUAccagcugaau | 351 | a*U*ucaGcUGguacCcCucggg*c*u | 498 |
| 100546 | c*g*agggGuACCagcugaauua | 352 | u*A*auuCaGCugguAcCccucg*g*g | 499 |
| 100547 | g*g*uaccAgCUGaauuacugca | 353 | u*G*cagUaAUucagCuGguacc*c*c | 500 |
| 100548 | g*u*accaGcUGAauuacugcag | 354 | C*U*gcaGuAAuucaGcUgguac*c*c | 501 |
| 100549 | u*a*ccagCuGAAuuacugcagu | 355 | a*C*ugcAgUAauucAgCuggua*c*c | 502 |
| 100550 | a*c*cagcUgAAUuacugcagug | 356 | C*A*cugCaGUaauuCaGcuggu*a*c | 503 |
| 100551 | c*c*agcuGaAUUacugcagugg | 357 | c*C*acuGcAGuaauUcAgcugg*u*a | 504 |
| 100552 | a*g*cugaAuUACugcagugggc | 358 | g*C*ccaCuGCaguaAuUcagcu*g*g | 505 |
| 100553 | g*c*ugaaUuACUgcagugggca | 359 | u*G*cccAcUGcaguAaUucagc*u*g | 506 |
| 100554 | c*u*gaauUaCUGcagugggcag | 360 | c*U*gccCaCUgcagUaAuucag*c*u | 507 |
| 100555 | a*u*uacuGcAGUgggcagugcc | 361 | g*G*cacUgCCcacuGcAguaau*u*c | 508 |
| 100556 | g*g*cauuGcUGCcucuuuccau | 362 | a*U*ggaAaGAggcaGcAaugcc*u*g | 509 |
| 100557 | g*c*auugCuGCCucuuuccauu | 363 | a*A*uggAaAGaggcAgCaaugc*c*u | 510 |
| 100558 | c*u*cuuuCcAUUcugccgucuu | 364 | a*A*gacGgCAgaauGgAaagag*g*c | 511 |
| 100559 | u*c*agccUcCUCaaagccaaca | 365 | u*G*uugGcUUugagGaGgcuga*a*g | 512 |
| 100560 | c*a*gccuCcUCAaagccaacaa | 366 | u*U*guuGgCUuugaGgAggcug*a*a | 513 |
| 100561 | u*c*cucaAaGCCaacaauccuu | 367 | a*A*ggaUuGUuggcUuUgagga*g*g | 514 |
| 100562 | u*c*ucucCuCUAccuggaucau | 368 | a*U*gauCcAGguagAgGagaga*g*a | 515 |
| 100563 | u*c*uccuCuACCuggaucauaa | 369 | u*U*augAuCCagguAgAggaga*g*a | 516 |
| 100564 | c*u*ccucUaCCUggaucauaau | 370 | a*U*uauGaUCcaggUaGaggag*a*g | 517 |
| 100565 | u*a*ccugGaUCAuaauggcaau | 371 | a*U*ugcCaUUaugaUcCaggua*g*a | 518 |
| 100566 | c*c*uggaUcAUAauggcaaugu | 372 | a*C*auuGcCAuuauGaUccagg*u*a | 519 |
| 100567 | a*u*cauaAuGGCaaugugguca | 373 | u*G*accAcAUugccAuUaugau*c*c | 520 |
| 100568 | u*c*auaaUgGCAauguggucaa | 374 | u*U*gacCaCAuugcCaUuauga*u*c | 521 |
| 100569 | c*a*uaauGgCAAuguggucaag | 375 | c*U*ugaCcACauugCcAuuaug*a*u | 522 |
| 100570 | a*u*aaugGcAAUguggucaaga | 376 | u*C*uugAcCAcauuGcCauuau*g*a | 523 |
| 100571 | u*a*auggCaAUGuggucaagac | 377 | g*U*cuuGaCCacauUgCcauua*u*g | 524 |
| 100572 | a*a*uggcAaUGUggucaagacg | 378 | c*G*ucuUgACcacaUuGccauu*a*u | 525 |
| 100573 | c*a*auguGgUCAagacggaugu | 379 | a*C*aucCgUCuugaCcAcauug*c*c | 526 |
| 100574 | c*a*agacGgAUGugccagauau | 380 | a*U*aucUgGCacauCcGucuug*a*c | 527 |
| 100575 | g*a*ggccUgUGGcugcagcuag | 381 | c*U*agcUgCAgccaCaGgccuc*c*a | 528 |
| 100576 | a*g*gccuGuGGCugcagcuagc | 382 | g*C*uagCuGCagccAcAggccu*c*c | 529 |
| 100577 | g*g*ccugUgGCUgcagcuagca | 383 | u*G*cuaGcUGcagcCaCaggcc*u*c | 530 |
| 100578 | g*c*cuguGgCUGcagcuagcaa | 384 | u*U*gcuAgCUgcagCcAcaggc*c*u | 531 |
| 100579 | c*c*ugugGcUGCagcuagcaag | 385 | c*U*ugcUaGCugcaGcCacagg*c*c | 532 |
| 100580 | g*c*uuugGaGUGaagagaccaa | 386 | u*U*gguCuCUucacUcCaaagc*c*c | 533 |
| 100581 | c*a*accaCcUGGcaauaugacu | 387 | a*G*ucaUaUUgccaGgUgguug*u*u | 534 |
| 100582 | a*c*caccUgGCAauaugacuca | 388 | u*G*aguCaUAuugcCaGguggu*u*g | 535 |
| 100583 | c*a*ccugGcAAUaugacucacu | 389 | a*G*ugaGuCAuauuGcCaggug*g*u | 536 |
| 100584 | g*g*acccAaAUGggcacuuucu | 390 | a*G*aaaGuGCccauUuGggucc*c*a | 537 |
| 100585 | c*c*caaaUgGGCacuuucuugu | 391 | a*C*aagAaAGugccCaUuuggg*u*c | 538 |
| 100586 | c*a*aaugGgCACuuucuugucu | 392 | a*G*acaAgAAagugCcCauuug*g*g | 539 |
| 100587 | g*g*cacuUuCUUgucugagacu | 393 | a*G*ucuCaGAcaagAaAgugcc*c*a | 540 |
| 100588 | c*a*cuuuCuUGUcugagacucu | 394 | a*G*aguCuCAgacaAgAaagug*c*c | 541 |
| 100589 | c*u*ugucUgAGAcucuggcuua | 395 | u*A*agcCaGAgucuCaGacaag*a*a | 542 |
| 100590 | a*g*ggaaGgCAGagaaaaauua | 396 | u*A*auuUuUCucugCcUucccu*c*c | 543 |
| 100591 | g*g*gaagGcAGAgaaaaauuac | 397 | g*U*aauUuUUcucuGcCuuccc*u*c | 544 |
| 100592 | a*g*ccucUcCCAagaugagaaa | 398 | u*U*ucuCaUCuuggGaGaggcu*a*a | 545 |
| 100593 | c*c*ucucCcAAGaugagaaagu | 399 | a*C*uuuCuCAucuuGgGagagg*c*u | 546 |
| 100594 | c*u*cccaAgAUGagaaaguccu | 400 | a*G*gacUuUCucauCuUgggag*a*g | 547 |
| 100595 | g*g*ggagGaGGAagcagauaga | 401 | u*C*uauCuGCuuccUcCucccc*u*c | 548 |
| 100596 | g*g*gaggAgGAAgcagauagau | 402 | a*U*cuaUcUGcuucCuCcuccc*c*u | 549 |
| 100597 | c*a*gaaaCaGGAgucaggaaaa | 403 | u*U*uucCuGAcuccUgUuucug*g*g | 550 |
| 100598 | g*c*acuaAgCCUaagaaguucc | 404 | g*G*aacUuCUuaggCuUagugc*c*u | 551 |
| 100599 | c*c*cacuGgGAGacaagcauuu | 405 | a*A*augCuUGucucCcAguggg*u*c | 552 |
| 100600 | c*c*acugGgAGAcaagcauuua | 406 | u*A*aauGcUUgucuCcCagugg*g*u | 553 |
| 100601 | a*c*ugggAgACAagcauuuaua | 407 | u*A*uaaAuGCuuguCuCccagu*g*g | 554 |
| 100602 | u*g*ggagAcAAGcauuuauacu | 408 | a*G*uauAaAUgcuuGuCuccca*g*u | 555 |
| 100603 | g*g*gagaCaAGCauuuauacuu | 409 | a*A*guaUaAAugcuUgUcuccc*a*g | 556 |
| 100604 | g*a*caagCaUUUauacuuucuu | 410 | a*A*gaaAgUAuaaaUgCuuguc*u*c | 557 |
| 100605 | g*a*gccaCcGCGccuggcuuau | 411 | a*U*aagCcAGgcgcGgUggcuc*a*c | 558 |
| 100606 | c*c*accgCgCCUggcuuauacu | 412 | a*G*uauAaGCcaggCgCggugg*c*u | 559 |
| 100607 | a*c*cgcgCcUGGcuuauacuuu | 413 | a*A*aguAuAAgccaGgCgcggu*g*g | 560 |
| 100608 | c*g*cgccUgGCUuauacuuucu | 414 | a*G*aaaGuAUaagcCaGgcgcg*g*u | 561 |
| 100609 | g*c*gccuGgCUUauacuuucuu | 415 | a*A*gaaAgUAuaagCcAggcgc*g*g | 562 |
| 100610 | c*g*ccugGcUUAuacuuucuua | 416 | u*A*agaAaGUauaaGcCaggcg*c*g | 563 |
| 100611 | g*c*cuggCuUAUacuuucuuaa | 417 | u*U*aagAaAGuauaAgCcaggc*g*c | 564 |
| 100612 | c*c*uggcUuAUAcuuucuuaau | 418 | a*U*uaaGaAAguauAaGccagg*c*g | 565 |
| 100613 | u*g*gcuuAuACUuucuuaauaa | 419 | u*U*auuAaGAaaguAuAagcca*g*g | 566 |
| 100614 | g*g*cuuaUaCUUucuuaauaaa | 420 | u*U*uauUaAGaaagUaUaagcc*a*g | 567 |
| 100615 | c*u*uauaCuUUCuuaauaaaaa | 421 | u*U*uuuAuUAagaaAgUauaag*c*c | 568 |
| 100616 | a*g*ggguGuCCAcaaagucaaa | 422 | u*U*ugaCuUUguggAcAccccu*g*a | 569 |
| 100617 | g*g*ggugUcCACaaagucaaag | 423 | c*U*uugAcUUugugGaCacccc*u*g | 570 |
| 100618 | u*c*auaaUaAUAcuaacauguu | 424 | a*A*cauGuUAguauUaUuauga*a*a | 571 |
| 100619 | a*c*uaacAuGUUauuugccuuu | 425 | a*A*aggCaAAuaacAuGuuagu*a*u | 572 |
| 100620 | g*u*uauuUgCCUuuugaauucu | 426 | a*G*aauUcAAaaggCaAauaac*a*u | 573 |
| 100621 | u*g*ccuuUuGAAuucucauuau | 427 | a*U*aauGaGAauucAaAaggca*a*a | 574 |
| 100622 | g*c*cuuuUgAAUucucauuauc | 428 | g*A*uaaUgAGaauuCaAaaggc*a*a | 575 |
| 100623 | c*c*uuuuGaAUUcucauuaucu | 429 | a*G*auaAuGAgaauUcAaaagg*c*a | 576 |
| 100624 | c*u*uuugAaUUCucauuaucuu | 430 | a*A*gauAaUGagaaUuCaaaag*g*c | 577 |
| 100625 | u*g*aauuCuCAUuaucuuaaaa | 431 | u*U*uuaAgAUaaugAgAauuca*a*a | 578 |
| 100626 | g*a*auucUcAUUaucuuaaaau | 432 | a*U*uuuAaGAuaauGaGaauuc*a*a | 579 |
| 100627 | c*c*guguGaCAUgugauuacau | 433 | a*U*guaAuCAcaugUcAcacgg*c*c | 580 |
| 100628 | u*g*ugacAuGUGauuacaucau | 434 | a*U*gauGuAAucacAuGucaca*c*g | 581 |
| 100629 | u*g*acauGuGAUuacaucaucu | 435 | a*G*augAuGUaaucAcAuguca*c*a | 582 |
| 100630 | g*a*caugUgAUUacaucaucuu | 436 | a*A*gauGaUGuaauCaCauguc*a*c | 583 |
| 100631 | u*c*uuucUgACAucauuguuaa | 437 | u*U*aacAaUGauguCaGaaaga*u*g | 584 |
| 100632 | c*u*uucuGaCAUcauuguuaau | 438 | a*U*uaaCaAUgaugUcAgaaag*a*u | 585 |
| 100633 | g*a*caucAuUGUuaauggaaug | 439 | C*A*uucCaUUaacaAuGauguc*a*g | 586 |
| 100634 | g*u*uaauGgAAUgugugcuugu | 440 | a*C*aagCaCAcauuCcAuuaac*a*a | 587 |

Abbreviation: (*) = PS bond; (-) = PO bond; lower case = 2′-OMe; capital = 2′-F; rX = RNA; dX = DNA; invAb = inverted abasic; Tgn = thymidine-glycol nucleic acid (GNA) S-Isomer; i = inosine; invdN = inverted deoxyribonucleotide (3′-3′ linked nucleotide or 5′-5′ linked nucleotide) Agn = adenosine-glycol nucleic acid (GNA) S-Isomer; Cgn = cytidine-glycol nucleic acid (GNA) S-Isomer; VP = 5′-E-Vinyl-phosphonate.

TABLE 3
Results of single dose screens at 10 nM and 0.1
nM in Huh7 cells using the selected modified INHBE
siRNAs shown as % inhibition Average and SD.

| | 10 nM | 10 nM | 0.1 nM | 0.1 nM |
|---|---|---|---|---|
| Compound ID | MEAN | SD | Mean | SD |
| 100488 | −5.35 | 5.35 | −38.35 | 54.87 |
| 100489 | 37.83 | 9.72 | −25.09 | 23.01 |
| 100490 | 33.23 | 4.19 | 2.17 | 17.95 |
| 100491 | 33.01 | 5.79 | −2.32 | 7.93 |
| 100492 | 62.61 | 3.47 | 37.88 | 14.26 |
| 100493 | 52.64 | 4.18 | 5.94 | 12.72 |
| 100494 | 66.97 | 5.86 | −8.14 | 13.30 |
| 100495 | 17.99 | 18.55 | −38.42 | 5.00 |
| 100496 | −1.87 | 2.86 | −12.67 | 65.05 |
| 100497 | 11.41 | 8.24 | −27.87 | 4.39 |
| 100498 | 6.01 | 9.66 | 2.04 | 12.74 |
| 100499 | 8.46 | 26.75 | −35.43 | 8.51 |
| 100500 | 41.01 | 12.56 | 28.92 | 19.85 |
| 100501 | 65.34 | 2.08 | 40.38 | 7.72 |
| 100502 | 35.53 | 10.67 | 45.79 | 8.53 |
| 100503 | −5.01 | 7.58 | −22.95 | 6.70 |
| 100504 | −0.27 | 15.93 | −7.15 | 11.52 |
| 100505 | 60.17 | 2.80 | −7.48 | 14.07 |
| 100506 | 66.21 | 0.85 | 9.93 | 4.35 |
| 100507 | 72.48 | 2.01 | 20.90 | 3.77 |
| 100508 | 46.83 | 1.89 | 26.11 | 6.19 |
| 100509 | 81.14 | 4.63 | 38.85 | 18.83 |
| 100510 | 36.30 | 22.83 | 13.27 | 37.06 |
| 100511 | 2.34 | 12.26 | −4.98 | 9.67 |
| 100512 | −10.95 | 3.33 | −17.49 | 2.44 |
| 100513 | 2.67 | 2.85 | −3.68 | 2.34 |
| 100514 | −2.59 | 9.74 | −3.02 | 10.27 |
| 100515 | 27.36 | 12.13 | 7.48 | 8.08 |
| 100516 | 9.59 | 11.30 | 13.53 | 11.12 |
| 100517 | 23.71 | 9.63 | 23.04 | 28.34 |
| 100518 | 36.59 | 8.04 | 2.30 | 7.98 |
| 100519 | 64.14 | 3.60 | 35.23 | 15.42 |
| 100520 | 33.42 | 3.20 | −4.67 | 20.21 |
| 100521 | 15.52 | 13.25 | −23.84 | 3.48 |
| 100522 | −33.65 | 11.75 | −11.62 | 10.71 |
| 100523 | 7.10 | 9.53 | −1.90 | 14.80 |
| 100524 | −7.80 | 4.06 | −10.19 | 8.80 |
| 100525 | −4.53 | 4.74 | −6.45 | 10.07 |
| 100526 | −21.17 | 15.69 | −33.23 | 20.13 |
| 100527 | −45.62 | 22.22 | −25.92 | 18.84 |
| 100528 | −26.98 | 12.15 | −5.43 | 22.85 |
| 100529 | −3.72 | 4.98 | −13.90 | 4.38 |
| 100530 | 4.07 | 5.95 | −0.27 | 9.78 |
| 100531 | 22.47 | 6.13 | 16.97 | 7.18 |
| 100532 | 15.61 | 7.43 | 19.46 | 17.58 |
| 100533 | 26.41 | 7.79 | 9.36 | 8.87 |
| 100534 | 34.02 | 3.29 | −11.20 | 6.44 |
| 100535 | 85.19 | 3.13 | 34.61 | 5.39 |
| 100536 | −3.01 | 18.07 | −25.60 | 10.69 |
| 100537 | 8.81 | 16.07 | −30.49 | 19.16 |
| 100538 | −2.31 | 22.08 | −5.04 | 13.00 |
| 100539 | 21.46 | 13.90 | 4.71 | 18.36 |
| 100540 | 45.70 | 28.80 | 0.07 | 8.28 |
| 100541 | 49.25 | 8.91 | 10.43 | 7.94 |
| 100542 | 33.24 | 11.87 | −0.17 | 11.73 |
| 100543 | 41.67 | 14.86 | 8.09 | 27.00 |
| 100544 | 4.52 | 5.12 | 2.61 | 11.66 |
| 100545 | 28.05 | 31.74 | 14.39 | 42.11 |
| 100546 | 37.79 | 12.09 | −1.95 | 12.26 |
| 100547 | −25.80 | 4.06 | −54.29 | 25.21 |
| 100548 | −12.11 | 21.78 | −49.30 | 7.75 |
| 100549 | 58.93 | 1.75 | −20.45 | 19.96 |
| 100550 | −4.66 | 6.98 | 2.61 | 8.97 |
| 100551 | 22.47 | 0.21 | 0.37 | 6.39 |
| 100552 | −16.76 | 27.81 | 0.37 | 13.86 |
| 100553 | −1.38 | 14.62 | −31.00 | 16.34 |
| 100554 | 6.08 | 28.04 | −12.79 | 19.47 |
| 100555 | 3.70 | 11.78 | −2.77 | 9.74 |
| 100556 | 44.87 | 2.94 | 16.18 | 2.60 |
| 100557 | 76.66 | 2.23 | 52.70 | 1.33 |
| 100558 | 62.62 | 4.14 | 34.69 | 3.68 |
| 100559 | 28.15 | 5.10 | 18.72 | 20.77 |
| 100560 | 37.94 | 22.04 | −6.69 | 23.88 |
| 100561 | 68.11 | 6.19 | 31.17 | 6.84 |
| 100562 | 26.76 | 1.80 | 25.79 | 12.14 |
| 100563 | 77.60 | 0.75 | 69.69 | 3.31 |
| 100564 | 70.51 | 0.56 | 47.60 | 4.97 |
| 100565 | 75.19 | 1.77 | 64.73 | 3.92 |
| 100566 | 47.23 | 12.69 | 23.51 | 5.52 |
| 100567 | 58.40 | 7.24 | 1.61 | 24.05 |
| 100568 | 73.44 | 0.72 | 36.93 | 3.46 |
| 100569 | 85.47 | 0.81 | 27.85 | 23.08 |
| 100570 | 63.92 | 2.80 | 39.92 | 9.93 |
| 100571 | 73.26 | 12.14 | 24.30 | 6.65 |
| 100572 | 58.31 | 4.40 | 14.67 | 6.54 |
| 100573 | 82.91 | 2.36 | 36.04 | 2.19 |
| 100574 | 80.59 | 0.59 | 60.99 | 6.06 |
| 100575 | 47.59 | 24.11 | 23.03 | 12.74 |
| 100576 | 34.93 | 25.86 | 26.51 | 33.73 |
| 100577 | 12.18 | 20.28 | 24.40 | 0.62 |
| 100578 | 57.62 | 2.89 | 30.66 | 4.41 |
| 100579 | 46.56 | 3.86 | 28.50 | 16.18 |
| 100580 | 77.56 | 3.09 | 62.93 | 3.55 |
| 100581 | 61.60 | 7.82 | 35.76 | 7.39 |
| 100582 | 35.56 | 11.61 | 14.33 | 3.68 |
| 100583 | 26.60 | 11.02 | −29.00 | 5.86 |
| 100584 | 49.62 | 10.36 | 39.66 | 5.41 |
| 100585 | 55.47 | 4.71 | 33.40 | 8.89 |
| 100586 | 57.59 | 946.65 | 57.48 | 2.91 |
| 100587 | 61.43 | 6.19 | 35.20 | 8.80 |
| 100588 | 74.82 | 0.61 | 43.78 | 5.03 |
| 100589 | 77.21 | 1.32 | 72.42 | 7.41 |
| 100590 | −10.86 | 8.29 | −8.61 | 10.34 |
| 100591 | 23.74 | 3.05 | −0.83 | 9.84 |
| 100592 | 44.68 | 1.51 | 17.87 | 5.46 |
| 100593 | 25.04 | 24.50 | 8.82 | 49.83 |
| 100594 | 56.17 | 11.48 | 10.22 | 55.97 |
| 100595 | 46.61 | 1.94 | 0.06 | 6.23 |
| 100596 | 26.00 | 8.58 | −4.84 | 25.31 |
| 100597 | 54.05 | 1.65 | 37.73 | 2.68 |
| 100598 | 53.90 | 7.21 | 29.24 | 6.23 |
| 100599 | 44.04 | 13.44 | 20.95 | 13.84 |
| 100600 | 58.54 | 5.93 | 52.44 | 5.82 |
| 100601 | 66.62 | 6.01 | 59.79 | 3.95 |
| 100602 | 54.88 | 13.17 | 44.11 | 5.65 |
| 100603 | 64.42 | 5.51 | 57.19 | 3.57 |
| 100604 | 81.15 | 1.68 | 70.42 | 5.87 |
| 100605 | −22.92 | 9.16 | −51.17 | 8.84 |
| 100606 | 33.44 | 6.94 | −51.72 | 16.18 |
| 100607 | 21.71 | 11.71 | −18.49 | 9.38 |
| 100608 | 48.27 | 3.33 | 31.48 | 5.29 |
| 100609 | 54.85 | 6.31 | 42.50 | 5.50 |
| 100610 | 45.02 | 3.84 | 60.83 | 5.59 |
| 100611 | 55.49 | 11.13 | 66.79 | 6.64 |
| 100612 | 74.28 | 3.53 | 67.82 | 7.71 |
| 100613 | 77.76 | 0.38 | 76.84 | 0.48 |
| 100614 | 70.48 | 1.17 | 66.71 | 4.23 |
| 100615 | 75.16 | 2.43 | 60.80 | 15.84 |
| 100616 | 24.73 | 15.95 | 48.26 | 7.56 |
| 100617 | 43.65 | 16.26 | 13.90 | 5.70 |
| 100618 | 66.45 | 4.43 | 55.43 | 25.80 |
| 100619 | 63.86 | 18.07 | 59.99 | 8.47 |
| 100620 | 64.18 | 3.56 | 42.90 | 11.82 |
| 100621 | 68.17 | 1.40 | 66.24 | 5.45 |
| 100622 | 43.81 | 6.21 | 15.94 | 12.38 |
| 100623 | 73.30 | 2.99 | 56.78 | 2.34 |
| 100624 | 71.82 | 4.23 | 45.80 | 2.40 |
| 100625 | 72.15 | 1.49 | 71.54 | 1.93 |
| 100626 | 46.27 | 5.87 | 60.75 | 4.49 |
| 100627 | 61.59 | 4.07 | 60.24 | 4.80 |
| 100628 | 70.82 | 2.90 | 63.11 | 2.26 |
| 100629 | 74.35 | 1.86 | 73.52 | 1.63 |
| 100630 | 62.37 | 1.45 | 37.68 | 9.75 |
| 100631 | −56.56 | 14.48 | −23.69 | 20.04 |
| 100632 | 62.92 | 0.50 | 48.87 | 5.08 |
| 100633 | 34.44 | 10.48 | 45.64 | 17.04 |
| 100634 | 18.76 | 15.92 | −7.97 | 14.05 |

### Example 2. In Vitro Dose-Response Screen in Huh7 Cell Line

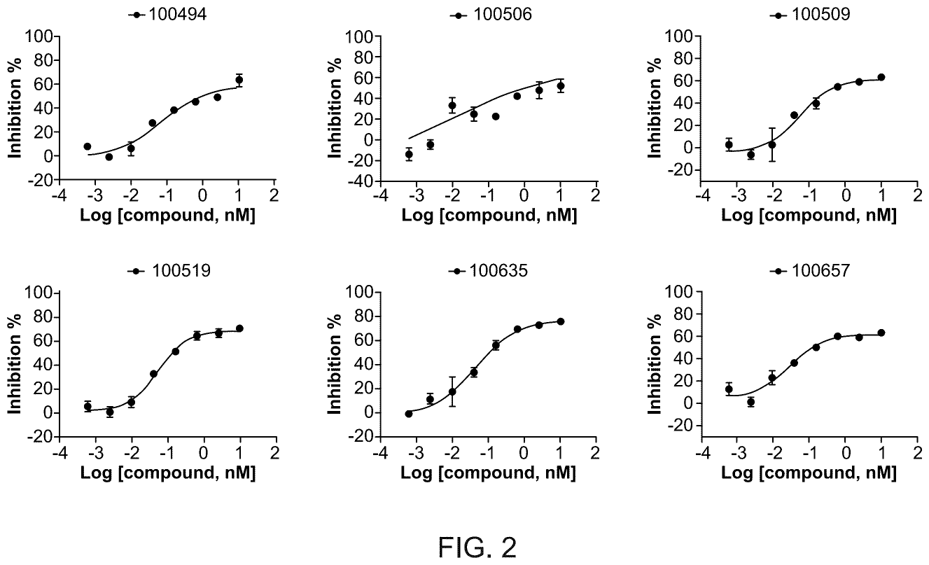

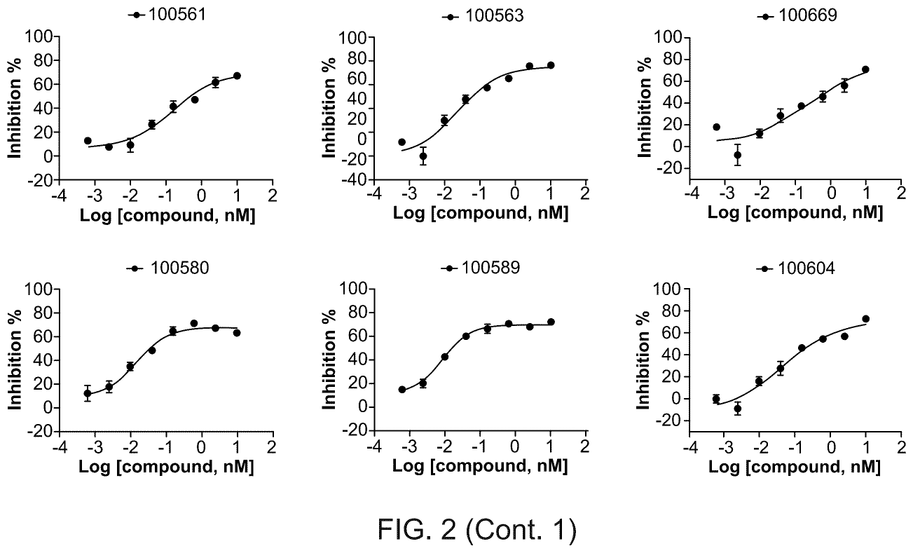

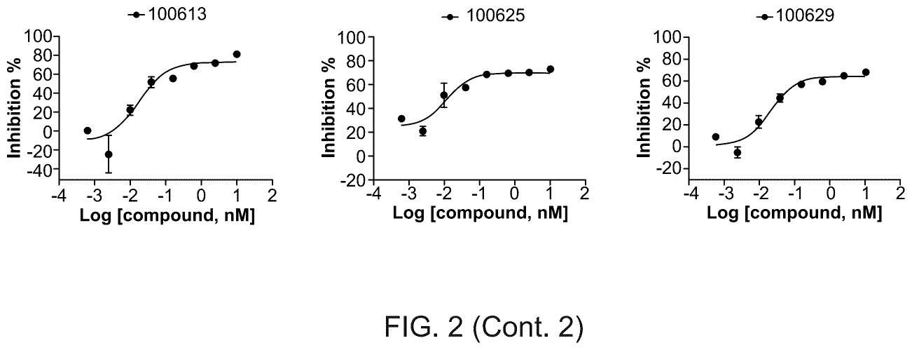

This example describes a screen of exemplary INHBE siRNA compounds in primary human hepatocytes (PHH) cells in a single dose screen at 70% nM, 33 nM, 1 IM, 3.7 nM, 1.2 nM, 0.412 nM, 0.137 nM, and 0.046 nM of the selected siRNA (Table 2). INHBE mRNA level was measured by quantitative PCR and normalized to GAPDH relative to mock treated control cells and the average KD and SD was determined. The data are presented as percent inhibition of INHBE mRNAs in the cells treated with siRNAs relatives to INHBE mRNA in the PBS control cells. Table 4 and FIG. 2 show the results of the in vitro dose-response INHBE siRNA screens.

TABLE 4
INHBE mRNA Max inhibition (%) and IC50
(nM) for exemplary INHBE siRNAs.

| Compound ID | IC50 (nM) | Max inhibition |
|---|---|---|
| 100494 | 0.879527207 | 60% |
| 100506 | 3.168648402 | 70% |
| 100509 | 0.288812209 | 62% |
| 100519 | 0.133171004 | 69% |
| 100535 | 0.102147368 | 78% |
| 100557 | 0.117147936 | 63% |
| 100561 | 0.447047125 | 71% |
| 100563 | 0.055169498 | 76% |
| 100569 | 0.720001201 | 79% |
| 100580 | 0.033891417 | 68% |
| 100589 | 0.015619441 | 70% |
| 100604 | 0.289690693 | 71% |
| 100613 | 0.042248546 | 74% |
| 100625 | 0.014322142 | 70% |
| 100629 | 0.057614467 | 64% |

### Example 3. In Vitro Dose-Response Screen in Primary Human Hepatocytes

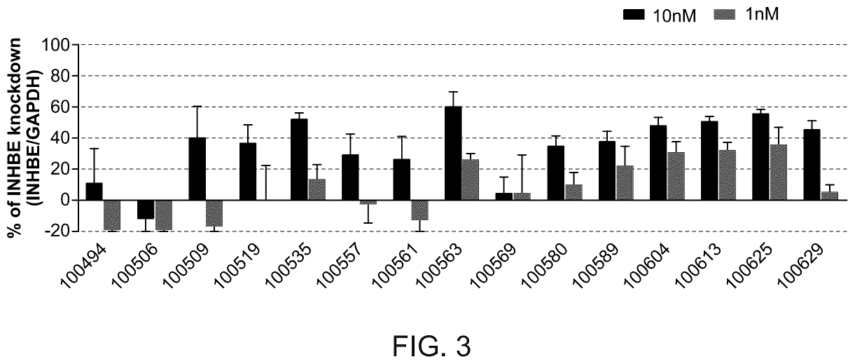

This example describes a screen of exemplary INHBE siRNA compounds in primary human hepatocytes (PHH) cells in a single dose screen at 10 nM and 1 nM of the selected siRNA (Table 2). INHBE mRNA level was measured by quantitative PCR and normalized to GAPDH relative to mock treated control cells and the mean KD and SD was determined. The data are presented as percent inhibition of INHBE mRNAs in the cells treated with siRNAs relative to INHBE mRNA in the PBS control cells. Table 5 and FIG. 3 show the results of the in vitro dose-response INHBE siRNA screens.

TABLE 5
Results of single dose screens at 10 nM and 0.1
nM in Huh7 cells using the selected modified INHBE
siRNAs shown as % inhibition MEAN and SD.

| Compound ID | 10 nM MEAN | 10 nM SD | 1 nM MEAN | 1 nM SD |
|---|---|---|---|---|
| 100494 | 10.7 | 22.4 | −22.5 | 4.0 |
| 100506 | −13.0 | 15.9 | −30.9 | 14.1 |
| 100509 | 40.1 | 19.4 | −16.7 | 10.5 |
| 100519 | 37.1 | 11.9 | 0.2 | 22.0 |
| 100535 | 51.9 | 4.1 | 13.7 | 9.8 |
| 100557 | 29.2 | 13.3 | −2.5 | 13.2 |
| 100561 | 26.8 | 14.3 | −13.5 | 6.6 |
| 100563 | 60.1 | 9.7 | 26.5 | 3.5 |
| 100569 | 4.4 | 10.2 | 5.1 | 23.5 |
| 100580 | 35.6 | 5.7 | 9.6 | 8.5 |
| 100589 | 37.8 | 6.4 | 21.7 | 12.9 |
| 100604 | 48.6 | 4.9 | 30.9 | 6.9 |
| 100613 | 50.8 | 3.1 | 31.8 | 5.9 |
| 100625 | 55.7 | 2.8 | 35.7 | 11.1 |
| 100629 | 45.7 | 5.6 | 5.1 | 5.0 |

### Example 4. Evaluation of Knockdown of Human INHBE with Select siRNAs in In Vivo Mouse Hydrodynamic Injection (HDI) Model

13 Exemplary siRNA compounds (Compounds 100635-100647) were tested for knockdown of human INHBE in a mouse model hydrodynamic injected with DNA plasmid encoding the full-length human INHBE transcript. Briefly, 6-7-week-old female BALB/c mice were subcutaneously injected with INHBE siRNA compounds from Table 6 at 1 mg/kg. Three days post injection, the mice were hydrodynamically injected with a DNA plasmid encoding the full-length human INHBE transcript. One day after the injection of the plasmid, liver samples were harvested and analyzed for INHBE mRNA expression relative to mice treated with the same volume of PBS. INHBE mRNA levels were measured by quantitative PCR and normalized to NEO gene included in the plasmid used to express INHBE. The data are presented as relative gene expression of INHBE mRNA in the liver relative to PBS treated animals.

The modified and unmodified sense and antisense strand sequences of Compounds 100329-100341 are summarized in Tables 6-7.

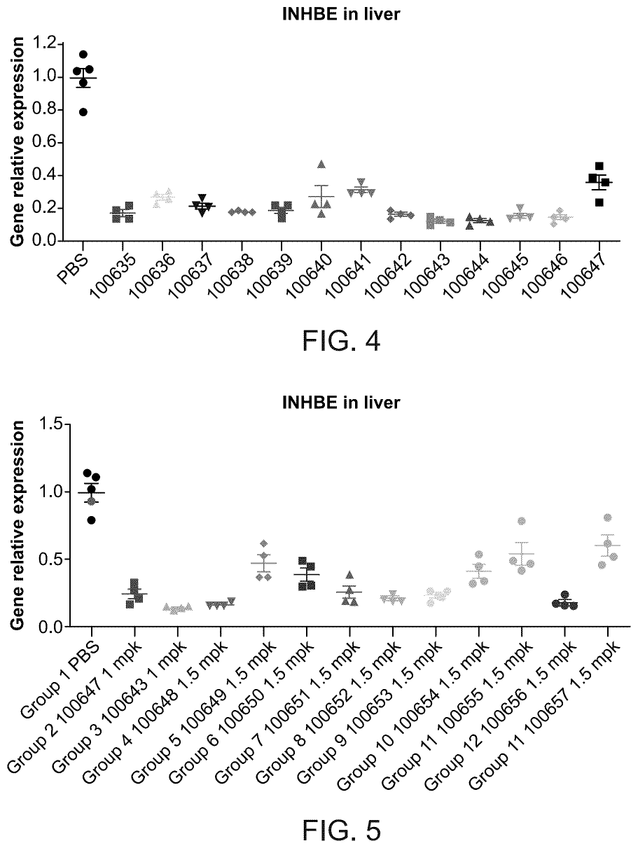

Table 8 and FIG. 4 show the results of single dose INHBE siRNA injection in INHBE BALB/c mice. The results show, that siRNA compounds 100635, 100638, 100639, 100642, 100643, 100644, 100645 and 100646 reduce INHBE expression by more than 80%. Additionally, compounds 100635-100646 all showed improved potency relative to siRNA compound 100647.

TABLE 6
Exemplary tri-GalNAc6 or L96 conjugated, modified INHBE siRNA
compounds, wherein the sense strand is conjugated to 5′-triGalNAc6, 3′-triGalNAc6, or 3′-
L96 targeting INHBE mRNA.

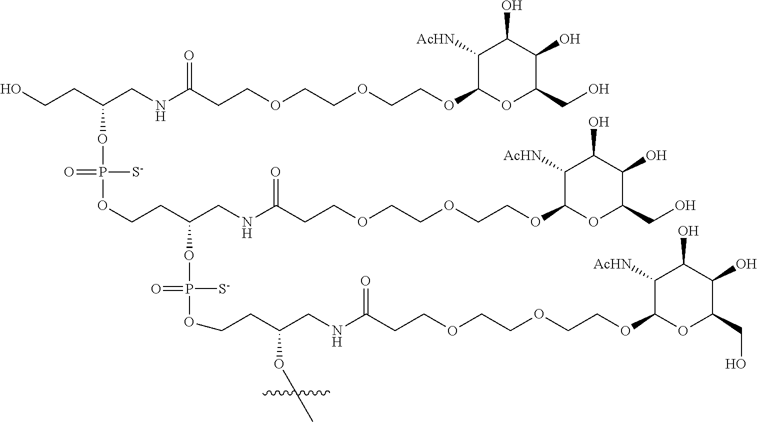 [SMILES: *O[C@H](CCOP(=O)([S-])O[C@H](CCOP(=O)([S-])O[C@H](CCO)CNC(=O)CCOCCOCCO[C@@H]1O[C@H](CO)[C@H](O)[C@H](O)[C@H]1NC(C)=O)CNC(=O)CCOCCOCCO[C@@H]1O[C@H](CO)[C@H](O)[C@H](O)[C@H]1NC(C)=O)CNC(=O)CCOCCOCCO[C@@H]1O[C@H](CO)[C@H](O)[C@H](O)[C@H]1NC(C)=O]

Tri-GalNAc6

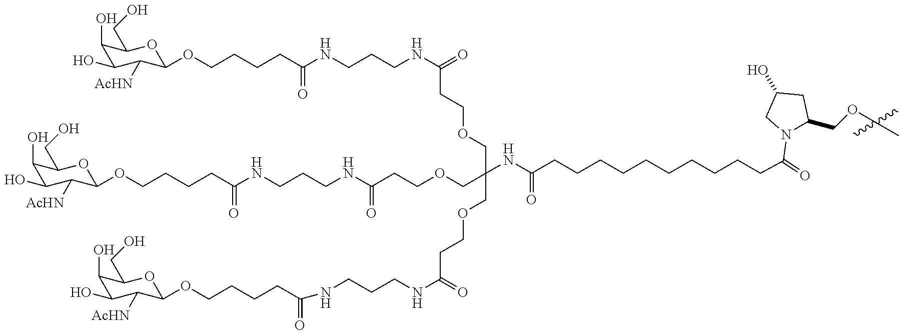 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

L96 ligand

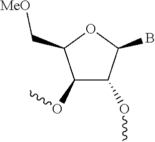

Modified siRNA nucleotide sugar, wherein B is the nucleotide base uracil (nucleotide
abbreviation: tmU) or cytosine (nucleotide abbreviation tmC)

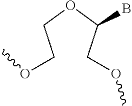

TNA analog, wherein B is a uracil base (abbreviation: utU) or an adenine base (abbreviation
utA)

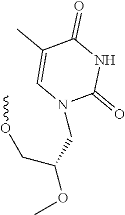

Glycol Nucleic Acid (GNA)

| Com- | | SEQ | | SEQ |
|---|---|---|---|---|
| pound | | ID | Antisense 5′-3′ | ID |
| ID | Sense 5′-3′ (modified) | NO: | (modified) | NO: |
| 100 | a*c*ucuUuGCUuGaggaucuu (L6) | 607 | va*A*gAuCcUcaagcAaAgagug*c*c | 616 |
| 635 | | | | |
| 100 | (L6)invdT*a*c*ucuUuGCUuGaggaucuu*u*u | 625 | va*A*gAuCcUcaagcAaAgagug*c*c | 632 |
| 636 | | | | |
| 100 | (L6) invdT*a*c*ucuUuGCUuGagga (utU)cuu*u*(tmU) | 626 | va*A*gAuCcUcaagcAaAgagug*c*c | 633 |
| 637 | | | | |
| 100 | a*c*ucuUuGCUuGagga(utU)cu(tmU)(L6) | 608 | va*A*gAuCcUcaagcAaAgagug*c*c | 617 |
| 638 | | | | |
| 100 | a *c*aagCaUUUaUacuuucuu(L6) | 609 | va*A*gaaAgUauaaaUgcuuguc*u*c | 618 |
| 639 | | | | |
| 100 | (L6) invdT*a*c*aagCaUUUaUacuuucuu*u*u | 627 | va*A*gaaAgUauaaaUgcuuguc*u*c | 634 |
| 640 | | | | |
| 100 | (L6) invdT*a*c*aagCaUUUaUacuu (tmU)cuu*u*(tmU) | 628 | va*A*gaaAgUauaaaUgcuuguc*u*c | 635 |
| 641 | | | | |
| 100 | a*c*aagCaUUUaUacuuucu(tmU)(L6) | 610 | va*A*gaaAgUauaaaUgcuuguc*u*c | 619 |
| 642 | | | | |
| 100 | g*g*cuuAuACUuUcuuaauaa(L6) | 611 | vu*U*aUuAaGaaaguAuAagcca*g*g | 620 |
| 643 | | | | |
| 100 | (L6) invdT*g*g*cuuAuACUuUcuuaauaa*u*u | 629 | vu*U*aUuAaGaaaguAuAagcca*g*g | 636 |
| 644 | | | | |
| 100 | (L6) invdT*g*g*cuuAuACUuUcuua (utA)uaa*u*(tmU) | 612 | vu*U*aUuAaGaaaguAuAagcca*g*g | 621 |
| 645 | | | | |
| 100 | g*g*cuuAuACUuUcuuaauaa (L6) | 630 | vu*U*a UuAaGaaaguAuAag(tmC)ca*g*g | 637 |
| 646 | | | | |
| 100 | C*u*gucaCaGACuccacuucau (L96) | 631 | A*U*gadAg (Tgn) ggagucUgUgacag*u*a | 638 |
| 647 | | | | |

Abbreviation: (*) = PS bond; lower case = 2′-OMe; capital = 2-F′; invdN = inverted deoxyribonucleotide (3′-3′ linked nucleotide or 5′-5′ linked nucleotide); v = 5′-E-Vinyl-phosphonate; (tmU) = siRNA nucleotide with a modified sugar and a uracil base; (utU) = a TNA analog with a uracil base, i.e., utU from above; (utA) = a TNA analog with a adenine base; i.e., utA from above; (L6) = tri-GalNAc6 ligand; (L96) = L96 ligand; (Tgn) = a thymidine-glycol nucleic acid, i.e., GNA from above; dX = DNA.

Glycol Nucleic Acid (GNA)

| Com- | | SEQ | Antisense | SEQ |
|---|---|---|---|---|
| pound | Sense 5′-3′ | ID | 5′-3′ | ID |
| ID | (modified) | NO: | (modified) | NO: |
| 100 | a*c*ucuUuGC | 607 | va*A*gAuCcU | 616 |
| 635 | UuGaggaucuu | | caagcAaAgag | |
| | (L6) | | ug*c*c | |
| 100 | (L6) invdT* | 625 | va*A*gAuCcU | 632 |
| 636 | a*c*ucuUuGC | | caagcAaAgag | |
| | UuGaggaucuu | | ug*c*c | |
| | *u*u | | | |
| 100 | (L6) invdT* | 626 | va*A*gAuCcU | 633 |
| 637 | a*c*ucuUuGC | | caagcAaAgag | |
| | UuGagga | | ug*c*c | |
| | (utU) | | | |
| | cuu*u* | | | |
| | (tmU) | | | |
| 100 | a*c*ucuUuGC | 608 | va*A*gAuCcU | 617 |
| 638 | UuGagga | | caagcAaAgag | |
| | (utU) cu | | ug*c*c | |
| | (tmU) (L6) | | | |
| 100 | a*c*aagCaUU | 609 | va*A*gaaAgU | 618 |
| 639 | UaUacuuucuu | | auaaaUgcuug | |
| | (L6) | | uc*u*c | |
| 100 | (L6) invdT* | 627 | va*A*gaaAgU | 634 |
| 640 | a*c*aagCaU | | auaaaUgcuug | |
| | UUaUacuuuc | | uc*u*c | |
| | uu*u*u | | | |
| 100 | (L6) invdT* | 628 | va*A*gaaAgU | 635 |
| 641 | a*c*aagCaUU | | auaaaUgcuug | |
| | UaUacuu | | uc*u*c | |
| | (tmU) | | | |
| | cuu*u* | | | |
| | (tmU) | | | |
| 100 | a*c*aagCaUU | 610 | va*A*gaaAgU | 619 |
| 642 | UaUacuuucu | | auaaaUgcuug | |
| | (tmU) (L6) | | uc*u*c | |
| 100 | g*g*cuuAuAC | 611 | vu*U*aUuAaG | 620 |
| 643 | UuUcuuaauaa | | aaaguAuAagc | |
| | (L6) | | ca*g*g | |
| 100 | (L6) invdT* | 629 | vu*U*aUuAaG | 636 |
| 644 | g*g*cuuAuAC | | aaaguAuAagc | |
| | UuUcuuaauaa | | ca*g*g | |
| | *u*u | | | |
| 100 | (L6) invdT* | 612 | vu*U*aUuAaG | 621 |
| 645 | g*g*cuuAuAC | | aaaguAuAagc | |
| | UuUcuua | | ca*g*g | |
| | (utA) | | | |
| | uaa*u* | | | |
| | (tmU) | | | |
| 100 | g*g*cuuAuAC | 630 | vu*U*aUuAaG | 637 |
| 646 | UuUcuuaauaa | | aaaguAuAag | |
| | (L6) | | (tmC) ca*g | |
| | | | *g | |
| 100 | C*u*gucaCaG | 631 | A*U*gadAg | 638 |
| 647 | ACuccacuucau | | (Tgn) | |
| | (L96) | | ggagucUg | |
| | | | Ugacag*u*a | |

Abbreviation:
(*) = PS bond;
lower case = 2′-OMe;
capital = 2′-F;
invdN = inverted deoxyribonucleotide (3′-3′ linked nucleotide or 5′-5′ linked nucleotide);
v = 5′-E-Vinyl-phosphonate;
(tmU) = siRNA nucleotide with a modified sugar and a uracil base;
(utU) = a TNA analog with a uracil base, i.e., utU from above;
(utA) = a TNA analog with a adenine base, i.e., utA from above;
(L6) = tri-GalNAc6 ligand;
(L96) = L96 ligand;
(Tgn) = a thymidine-glycol nucleic acid, i.e., GNA from above;
dX = DNA.

TABLE 7
INHBE siRNA unmodified sequences

| | SEQ | | SEQ |
|---|---|---|---|
| | ID | Antisense | ID |
| Sense 5′-3′ | NO: | 5′-3′ | NO: |
| ACUCUUUGCUUGA | 589 | AAGAUCCUCAAGC | 598 |
| GGAUCUU | | AAAGAGUGCC | |
| TACUCUUUGCUUG | 639 | AAGAUCCUCAAGC | 646 |
| AGGAUCUUUU | | AAAGAGUGCC | |
| TACUCUUUGCUUG | 640 | AAGAUCCUCAAGC | 647 |
| AGGAUCUUUU | | AAAGAGUGCC | |
| ACUCUUUGCUUGA | 590 | AAGAUCCUCAAGC | 599 |
| GGAUCUU | | AAAGAGUGCC | |
| ACAAGCAUUUAUA | 591 | AAGAAAGUAUAAA | 600 |
| CUUUCUU | | UGCUUGUCUC | |
| TACAAGCAUUUAU | 641 | AAGAAAGUAUAAA | 648 |
| ACUUUCUUUU | | UGCUUGUCUC | |
| TACAAGCAUUUAU | 642 | AAGAAAGUAUAAA | 649 |
| ACUUUCUUUU | | UGCUUGUCUC | |
| ACAAGCAUUUAUA | 592 | AAGAAAGUAUAAA | 601 |
| CUUUCUU | | UGCUUGUCUC | |
| GGCUUAUACUUUC | 593 | UUAUUAAGAAAGU | 602 |
| UUAAUAA | | AUAAGCCAGG | |
| TGGCUUAUACUUU | 643 | UUAUUAAGAAAGU | 650 |
| CUUAAUAAUU | | AUAAGCCAGG | |
| TGGCUUAUACUUU | 594 | UUAUUAAGAAAGU | 603 |
| CUUAAUAAUU | | AUAAGCCAGG | |
| GGCUUAUACUUUC | 644 | UUAUUAAGAAAGU | 651 |
| UUAAUAA | | AUAAGCCAGG | |
| CUGUCACAGACUC | 645 | AUGAAGTGGAGUC | 652 |
| CACUUCAU | | UGUGACAGUA | |

TABLE 8
In vivo percent knockdown (KD) in HDI model.

| Compound ID | % KD in HDI Model |
|---|---|
| 100635 | 83 |
| 100636 | 73 |
| 100637 | 79 |
| 100638 | 82 |
| 100639 | 81 |
| 100640 | 72 |
| 100641 | 69 |
| 100642 | 84 |
| 100643 | 88 |
| 100644 | 87 |
| 100645 | 84 |
| 100646 | 85 |
| 100647 | 64 |

### Example 5. Evaluation of Knockdown of Human INHBE with Select siRNAs in In Vivo Mouse Hydrodynamic Injection (HDI) Model

12 Exemplary siRNA compounds (Compounds 100643 and 100647-100657) were tested for knockdown of human INHBE in a mouse model hydrodynamic injected with DNA plasmid encoding the full-length human INHBE transcript. Briefly, 6-7-week-old female BALB/c mice were subcutaneously injected with INHBE siRNA compounds from Table 9 at 1 or 1.5 mg/kg. Three days post injection, the mice were hydrodynamically injected with a DNA plasmid encoding the full-length human INHBE transcript. One day after the injection of the plasmid, liver samples were harvested and analyzed for INHBE mRNA expression relative to mice treated with the same volume of PBS. INHBE mRNA levels were measured by quantitative PCR and normalized to NEO gene included in the plasmid used to express INHBE. The data are presented as relative gene expression of INHBE mRNA in the liver relative to PBS treated animals.

The modified and unmodified sense and antisense strand sequences of Compounds 100643 and 100647-100657 are summarized in Tables 9-10.

FIG. 5 show the results of single dose INHBE siRNA injection in INHBE BALB/c mice. The results show, that siRNA compounds 100643, 100649, 100650, 100654, 100655, and 100657 showed improved potency relative to siRNA compound 100647.

TABLE 9
Exemplary tri-GalNAc6 or L96 conjugated, modified INHBE siRNA
compounds, wherein the sense strand is conjugated to 3′-triGalNAc6 or 3′-L96 targeting
INHBE mRNA.

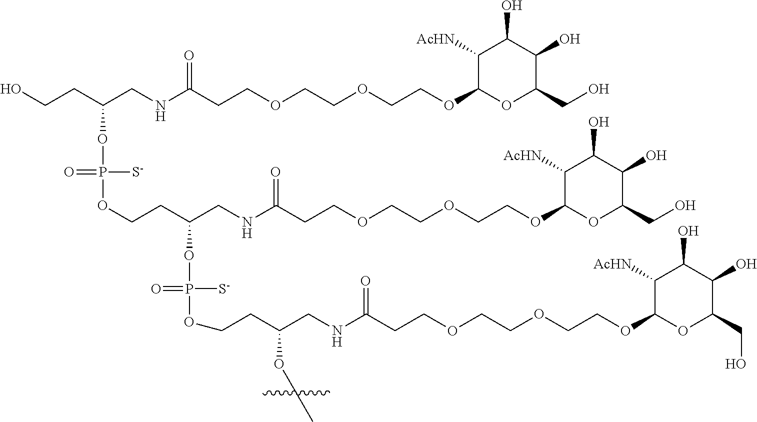 [SMILES: *O[C@H](CCOP(=O)([S-])O[C@H](CCOP(=O)([S-])O[C@H](CCO)CNC(=O)CCOCCOCCO[C@@H]1O[C@H](CO)[C@H](O)[C@H](O)[C@H]1NC(C)=O)CNC(=O)CCOCCOCCO[C@@H]1O[C@H](CO)[C@H](O)[C@H](O)[C@H]1NC(C)=O)CNC(=O)CCOCCOCCO[C@@H]1O[C@H](CO)[C@H](O)[C@H](O)[C@H]1NC(C)=O]

Tri-GalNAc6

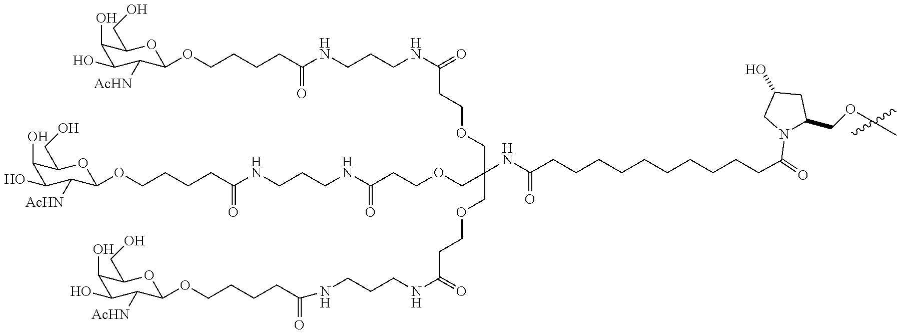 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

L96 ligand

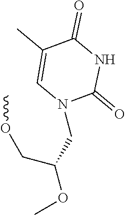

Glycol Nucleic Acid (GNA)

| Com- | | SEQ | | SEQ |
|---|---|---|---|---|
| pound | | ID | | ID |
| ID | Sense 5′-3′ (modified) | NO: | Antisense 5′-3′ (modified) | NO: |
| 100 | g*g*cuuAuACUuUcuuaauaa (L6) | 611 | vu*U*aUuAaGaaaguAuAagcca*g*g | 620 |
| 643 | | | | |
| 100 | C*u*gucaCaGACuccacuucau (L96) | 631 | A*U*gadAg(Tgn)ggagucUgUgacag*u*a | 638 |
| 647 | | | | |
| 100 | u*g*gcuuAuACUuucuuaauaa (L96) | 653 | u*U*auuAaGAaaguAuAagcca*g*g | 660 |
| 648 | | | | |
| 100 | u*u*ggagugAAGagaccaagau (L96) | 654 | vu*U*cUuGgUcucuuCaCuccaaag | 661 |
| 649 | | | | |
| 100 | u*c*uuuccaUUCugccgucuuc (L96) | 655 | vu*A*aGaCgGcagaaUgGaaagagg | 662 |
| 650 | | | | |
| 100 | g*g*agacaaGCAuuuauacuuu (L96) | 656 | vu*A*aGuAuAaaugcUuGucuccca | 663 |
| 651 | | | | |
| 100 | a*c*aagcauUUAuacuuucuuu (L96) | 613 | vu*A*aGaAaGuauaaAuGcuugucu | 622 |
| 652 | | | | |
| 100 | u*g*aagagaCCAagaugaaguu (L96) | 614 | vu*A*cUuCaUcuuggUcUcuucacu | 623 |
| 653 | | | | |
| 100 | g*a*gacaagCAUuuauacuuuc (L96) | 657 | vu*A*aAgUaUaaaugCuUgucuccc | 664 |
| 654 | | | | |
| 100 | a*a*ugggcaCUUucuugucuga (L96) | 615 | vu*C*aGaCaAgaaagUgCccauuug | 624 |
| 655 | | | | |
| 100 | a*g*agaccaAGAugaaguuucc (L96) | 658 | vu*G*aAaCuUcaucuuggucucuuc | 665 |
| 656 | | | | |
| 100 | a*a*gaaguuCCCugguuuuucc (L96) | 659 | vu*G*aAaAaCcagggAaCuucuuag | 666 |
| 657 | | | | |

Abbreviation: (*) = PS bond; lower case = 2′-OMe; capital = 2′-F; v = 5′-E-Vinyl-phosphonate; (L96) = L96 ligand; (Tgn) = a thymidine-glycol nucleic acid, i.e., GNA from above; dX = DNA

TABLE 10
INHBE siRNA unmodified sequences

| | SEQ | | SEQ |
|---|---|---|---|
| | ID | Antisense | ID |
| Sense 5′-3′ | NO: | 5′-3′ | NO: |
| ACUCUUUGCUUGA | 593 | AAGAUCCUCAAGC | 602 |
| GGAUCUU | | AAAGAGUGCC | |
| CUGUCACAGACUC | 645 | AUGAAGTGGAGUC | 652 |
| CACUUCAU | | UGUGACAGUA | |
| UGGCUUAUACUUU | 667 | UUAUUAAGAAAGU | 675 |
| CUUAAUAA | | AUAAGCCAGG | |
| UUGGAGUGAAGAG | 668 | UUCUUGGUCUCUU | 676 |
| ACCAAGAU | | CACUCCAAAG | |
| UCUUUCCAUUCUG | 670 | UAAGACGGCAGAA | 677 |
| CCGUCUUC | | UGGAAAGAGG | |
| GGAGACAAGCAUU | 671 | UAAGUAUAAAUGC | 678 |
| UAUACUUU | | UUGUCUCCCA | |
| ACAAGCAUUUAUA | 595 | UAAGAAAGUAUAA | 604 |
| CUUUCUUU | | AUGCUUGUCU | |
| UGAAGAGACCAAG | 596 | UACUUCAUCUUGG | 605 |
| AUGAAGUU | | UCUCUUCACU | |
| GAGACAAGCAUUU | 672 | UAAAGUAUAAAUG | 679 |
| AUACUUUC | | CUUGUCUCCC | |
| AAUGGGCACUUUC | 597 | UCAGACAAGAAAG | 606 |
| UUGUCUGA | | UGCCCAUUUG | |
| AGAGACCAAGAUG | 673 | UGAAACUUCAUCU | 680 |
| AAGUUUCC | | UGGUCUCUUC | |
| AAGAAGUUCCCUG | 674 | UGAAAAACCAGGG | 681 |
| GUUUUUCC | | AACUUCUUAG | |

### Example 6. Evaluation of Knockdown of INHBE with siRNAs in In Vivo Non-Human Primate Model

Two exemplary siRNA compounds, referred to herein as Compound A and Compound B, were selected from among the compounds listed in Table 6 and Table 9, and were tested for knockdown of INHBE in a non-human primate model. Briefly, 3-8 year old male cynomolgus monkeys were subcutaneously injected with one of either INHBE siRNA Compounds A or B at 5 mg/kg (n=2-3 monkeys per compound tested). Animals were subject to clinical observation twice daily after injection. Liver biopsies were obtained 4 days prior to injection, and 56 days after injection. Liver INHBE mRNA levels were measured by quantitative PCR an normalized to the day −4 level for each individual animals.

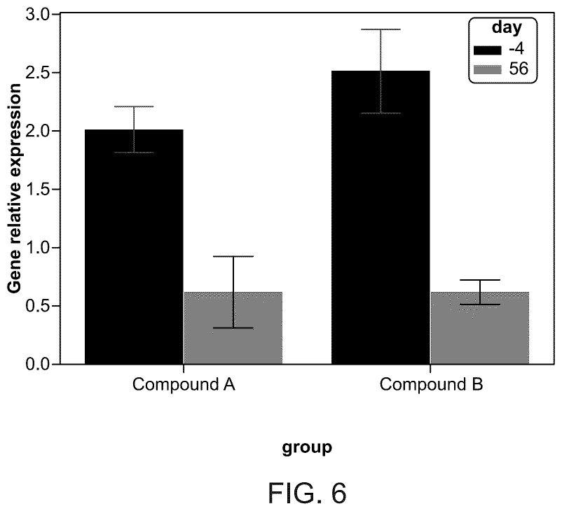

FIG. 6 shows the results of single dose INHBE siRNA injection in cynomolgus monkeys. The results show that 69% knockdown (Compound A) and 75% knockdown (Compound B) was observed in animals at day 56 after injection (FIG. 6).

### Example 7. In Vitro Activity and Dose-Response Screen in hTLR7, hTLR8, and hTLR9 Cells

This example evaluates the agonist activity of compounds in the cell-based human TLR Toll-like receptor (hTLR) reporter assay. Exemplary siRNA compounds 100647, 100635, 100638, 100639, 100642, 100643, and 10064 were tested using commercial cell-based hTLR7, hTLR8 and hTLR9 reporter assays. Briefly, assays were performed using a 4-fold dilution from 100 nM under 9 concentrations by transfection of HEK-293 cells in duplicate, and with a duration of treatment of 24 hours. R848 was purchased from a commercial vendor and used as the agonist for the hTRL7 and hTLR8 reporter assays. ODN 2006 was purchased from a commercial vendor and used as the agonist for the hTRL9 reporter assays.

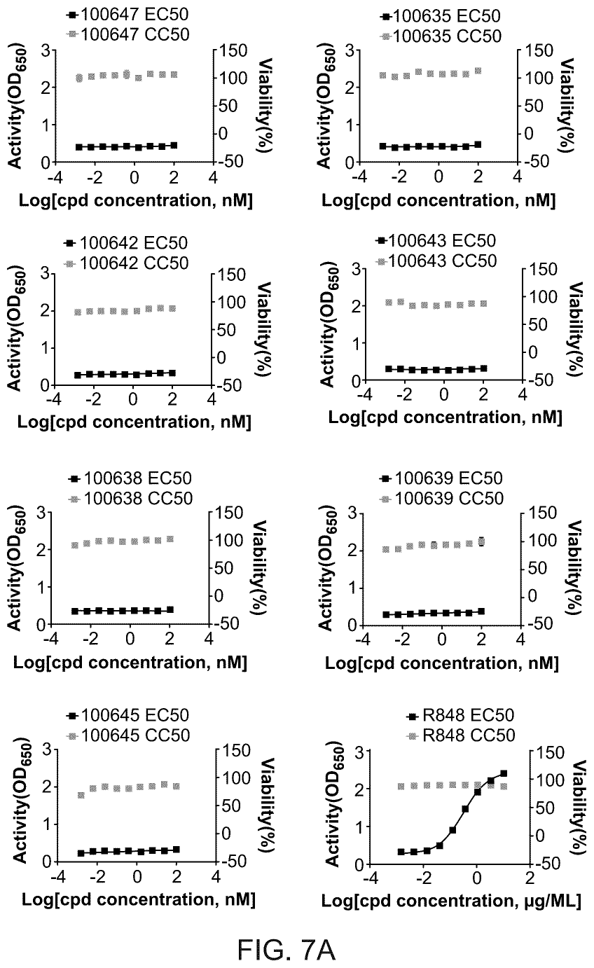

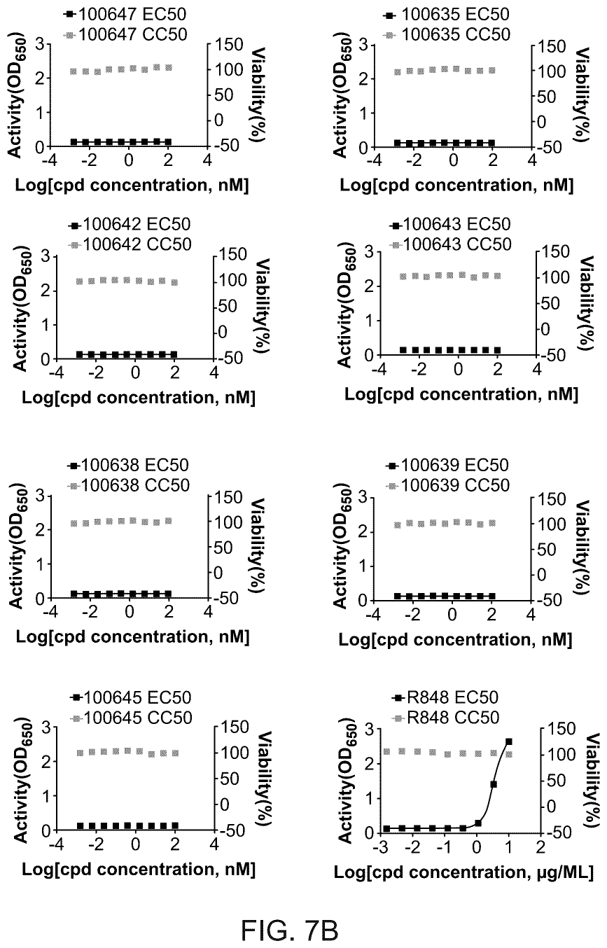

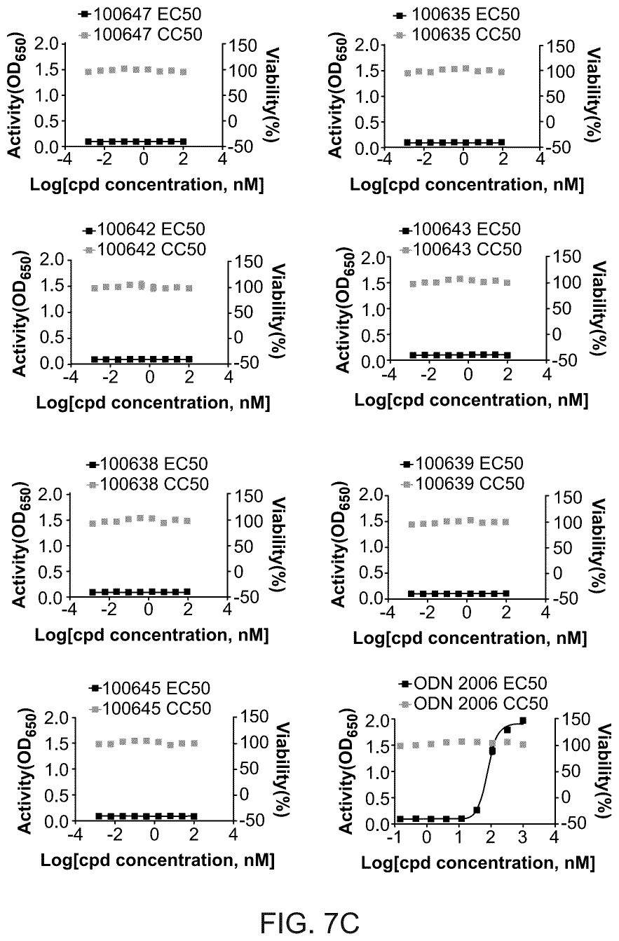

The data are presented as the level of activity in cells treated with test compounds as a fold change over the activity of unstimulated cells in the cell-based hTLR7, hTLR8, and hTLR9 reporter assays. Table 11 and FIGS. 7A-7C show the results of the cell-based hTLR7, hTLR8, and hTLR9 reporter assays. The results show that all tested siRNA compounds displayed no activity against hTLR7, hTLR8, and hTLR9 pathways.

TABLE 11
Activity of exemplary INHBE siRNA compounds in cell-
based hTLR7, hTLR8, and hTLR9 reporter assays.

| | EC₅₀ | | | CC₅₀ | | | |
|---|---|---|---|---|---|---|---|
| Compound ID | hTLR7 | hTLR8 | hTLR9 | hTLR7 | hTLR8 | hTLR9 | Unit |
| 100647 | >100 | >100 | >100 | >100 | >100 | >100 | nM |
| 100635 | >100 | >100 | >100 | >100 | >100 | >100 | nM |
| 100638 | >100 | >100 | >100 | >100 | >100 | >100 | nM |
| 100639 | >100 | >100 | >100 | >100 | >100 | >100 | nM |
| 100642 | >100 | >100 | >100 | >100 | >100 | >100 | nM |
| 100643 | >100 | >100 | >100 | >100 | >100 | >100 | nM |
| 100645 | >100 | >100 | >100 | >100 | >100 | >100 | nM |
| R848 | 0.924 | 9.863 | / | >10 | >10 | / | μM |
| ODN 2006 | / | / | 80.79 | / | / | >1000 | nM |

### Example 8. RNA Sequence Transcriptome Analysis in Primary Human Hepatocytes

This example evaluates the RNA sequence transcriptome in primary human hepatocytes to assess potential off-target risk of exemplary siRNA compounds 100647, 100635, 100638, 100639, 100642, 100643, and 100645. Briefly, primary human hepatocyte (PHH) cells were collected after 48 hours of treatment with of the exemplary siRNA compounds for RNA extrations, library construction, and sequencing.

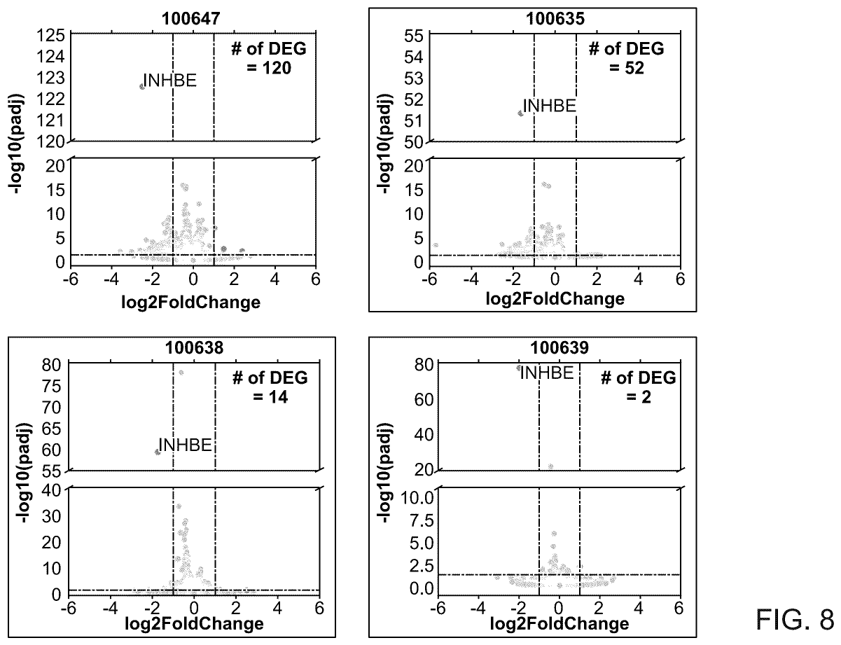

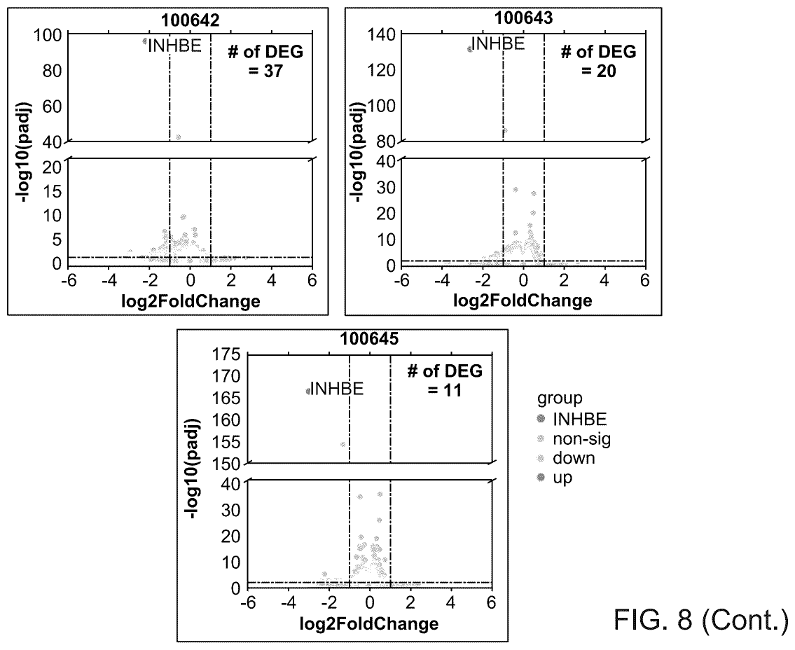

FIG. 8 shows the results of the RNA sequence transcriptome analysis in primary human hepatocytes treated with exemplary siRNA compounds. The data are presented as a volcano plot of differentially expresses genes (DEGs) among different groups, and show that INHBE is significantly down-regulated in all groups. Of all exemplary siRNA compounds tested, Compound 100639 returned the cleanest RNAseq result.

### Example 9. Non-GLP Mini Toxicology Study of Select siRNAs in In Vivo Mouse Model

Exemplary siRNA compounds 100635, 100642, and 100643 were evaluated via a non-GLP mini toxicology study in mice. Briefly, 7 week old male C57BL/6J mice (5 per group) were subcutaneously injected with a single 50 mg/kg dose of one of the 100635, 100642, and 100643 siRNA compounds (i.e., at day 0).

Blood was collected on day 0 (pre-dosing) and day 7. Tissues (liver and kidney) were collected on day 7 after termination.

Urine and blood samples harvested on day 0 (pre-dosing) were handled as follows. Plasma was snap frozen on snap frozen on dry ice upon collection, stored at −80 deg transferred for biochemistry analysis. Urine was stored at 4 degrees or −80 degree Celsius until transferred for biochemistry analysis.

Urine, blood, liver and kidney samples harvested on day 7 were handled as follows. Plasma: snap frozen on dry ice upon collection, stored at −80 deg transferred for biochemistry analysis Plasma was stored on ice until transferred for coagulation assays

Liver and kidney were fixed in 4% paraformaldehyde until transferred for histopathology evaluation.

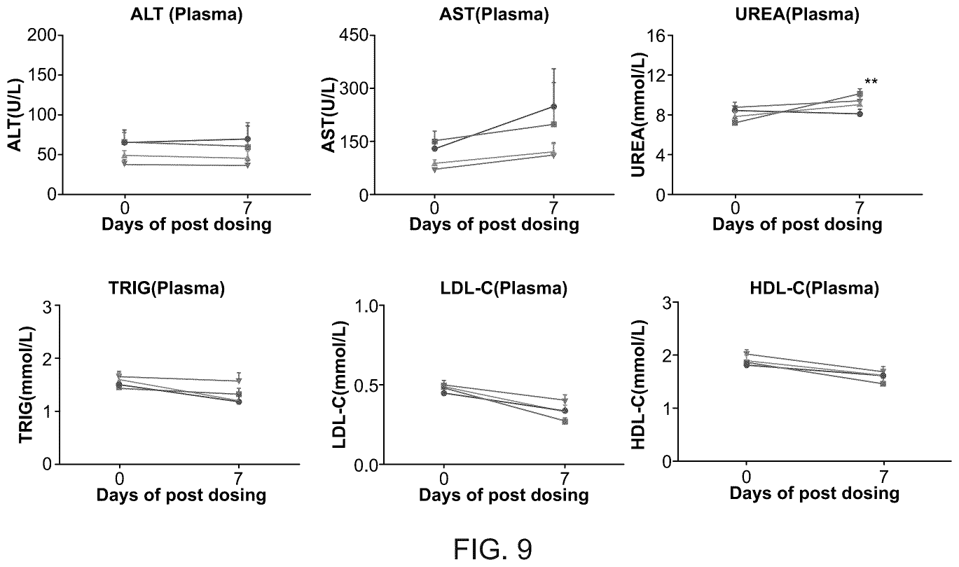

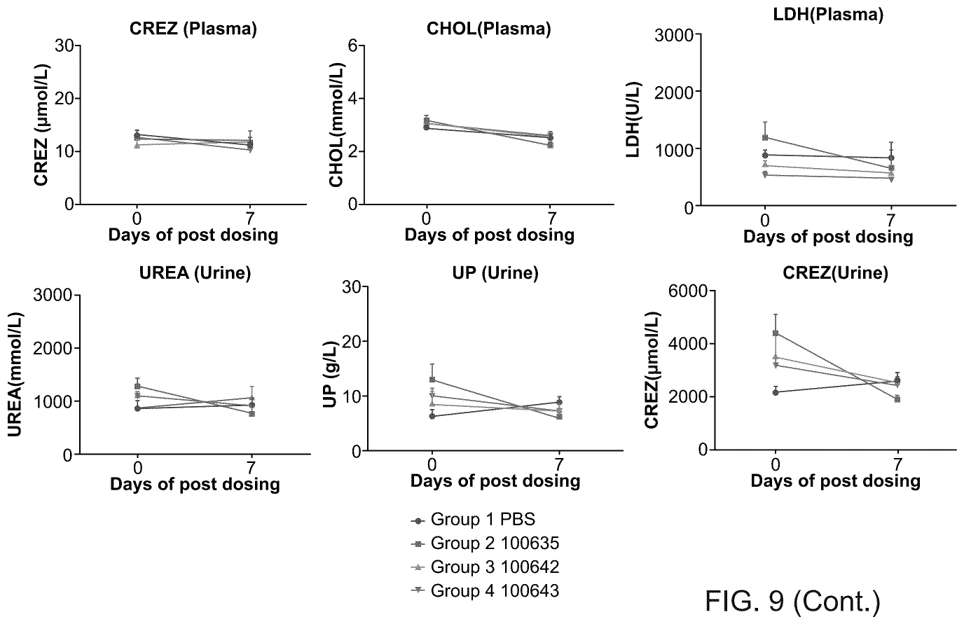

FIG. 9 and Table 12 shows the results of injecting C57BL/6J mice with a single 50 mg/kg dose of one of 100635, 100642, or 100643 siRNA compounds.

FIG. 9 shows the results of the mice biochemical tests over 7 days post dosing. Compared with the results of PBS control group, no significant difference was observed for the mean plasma concentration of alanine aminotransferase (ALT), aspartate aminotransferase (AST), triglycerides (TRIG), low-density lipoprotein cholesterol (LDL-C), high-density lipoprotein cholesterol (HDL-C), creatinine (CREZ), cholesterol (CHOL), and lactate dehydrogenase (LDH) of mice in the test compound groups. The mean plasma UREA concentration of mice in the 100635 group was significantly higher than that of mice in the PBS group on day 7. Compared with the results of PBS control group, no significant difference was observed for the mean urine concentration of urea (UREA), urine micro total protein (UP), and creatinine (CREZ) of mice in the test compound groups.

Table 12 shows the results of the liver and kidney pathology study over 7 days post dosing. The study results indicate no significant lesions (damage) in the experimental subjects. Observed changes in the PBS group may have be due to background lesions.

TABLE 12
Liver and kidney pathology study results.

| Group | Liver Pathological Diagnosis | Kidney Pathological Diagnosis |
|---|---|---|
| Group 1 | Inflammatory cells were occasionally seen | No significant lesion was found |
| PBS | around the blood vessels and liver margins | |
| 5 mL/kg, | Few inflammatory cell aggregation focis | No significant lesion was found |
| SC | along with hepatocellular degeneration | |
| | were found | |
| | Inflammatory cells were occasionally seen | No significant lesion was found |
| | in the portal area | |
| | Inflammatory cells were occasionally seen | No significant lesion was found |
| | No significant lesion was found | No significant lesion was found |
| Group 2 | No significant lesion was found | No significant lesion was found |
| Compound | No significant lesion was found | Interstitial cell hyperplasia were |
| 100635 | | occasionally seen |
| 50 mpk, | No significant lesion was found | No significant lesion was found |
| SC | No significant lesion was found | No significant lesion was found |
| | No significant lesion was found | No significant lesion was found |
| Group 3 | No significant lesion was found | No significant lesion was found |
| Compound | No significant lesion was found | No significant lesion was found |
| 100642 | No significant lesion was found | No significant lesion was found |
| 50 mpk, | Inflammatory cell aggregation focis were | No significant lesion was found |
| SC | occasionally seen in the portal area | |
| | Inflammatory cells were occasionally seen | No significant lesion was found |
| Group 4 | No significant lesion was found | No significant lesion was found |
| Compound | Inflammatory cell aggregation focis were | Basophilic tubule cells were |
| 100643 | occasionally seen in the portal area | occasionally seen |
| 50 mpk, | No significant lesion was found | No significant lesion was found |
| SC | No significant lesion was found | No significant lesion was found |
| | No significant lesion was found | Scattered inflammatory cells were |
| | | occasionally seen |

In conclusion, no significant differences were detected in the biochemical indexes tested, and no significant lesions or damage was observed by liver and kidney pathological diagnosis. Therefore, in general, the single dose subcutaneous administration of the test compounds 100635, 100642, or 100643 in mice was considered to be safe and no obvious toxicity was observed in this non-GLP study.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

SEQUENCE LISTING

| SEQ | | |
|---|---|---|
| ID | | |
| NO: | Description | Sequence |
| 1 | Sense 5′-3′ | GGGUCAAGCACAGCUAUCCAU |
| 2 | Sense 5′-3′ | CACAGCUAUCCAUCAGAUGAU |
| 3 | Sense 5′-3′ | CAGCUAUCCAUCAGAUGAUCU |
| 4 | Sense 5′-3′ | AGCUAUCCAUCAGAUGAUCUA |
| 5 | Sense 5′-3′ | CUAUCCAUCAGAUGAUCUACU |
| 6 | Sense 5′-3′ | UAUCCAUCAGAUGAUCUACUU |
| 7 | Sense 5′-3′ | CCAUCAGAUGAUCUACUUUCA |
| 8 | Sense 5′-3′ | CUACUUUCAGCCUUCCUGAGU |
| 9 | Sense 5′-3′ | GACAAUAGAAGACAGGUGGCU |
| 10 | Sense 5′-3′ | GCAGUGGUGUCUGCUGUCACU |
| 11 | Sense 5′-3′ | CUCAUUGGCCCCCAGCAAUCA |
| 12 | Sense 5′-3′ | CUCCUGUGGGGGCUCCAAACU |
| 13 | Sense 5′-3′ | CUGGAGCUAGCCAAGCAGCAA |
| 14 | Sense 5′-3′ | UGGAGCUAGCCAAGCAGCAAA |
| 15 | Sense 5′-3′ | GGAGCUAGCCAAGCAGCAAAU |
| 16 | Sense 5′-3′ | GAGCUAGCCAAGCAGCAAAUC |
| 17 | Sense 5′-3′ | AGCUAGCCAAGCAGCAAAUCC |
| 18 | Sense 5′-3′ | GCUAGCCAAGCAGCAAAUCCU |
| 19 | Sense 5′-3′ | UAGCCAAGCAGCAAAUCCUGG |
| 20 | Sense 5′-3′ | GCCAAGCAGCAAAUCCUGGAU |
| 21 | Sense 5′-3′ | UGACCAGUCGUCCCAGAAUAA |
| 22 | Sense 5′-3′ | ACCAGUCGUCCCAGAAUAACU |
| 23 | Sense 5′-3′ | CGUCCCAGAAUAACUCAUCCU |
| 24 | Sense 5′-3′ | CGCUGACCAGAGCCCUCCGGA |
| 25 | Sense 5′-3′ | GCUGACCAGAGCCCUCCGGAG |
| 26 | Sense 5′-3′ | CUGACCAGAGCCCUCCGGAGA |
| 27 | Sense 5′-3′ | UGACCAGAGCCCUCCGGAGAC |
| 28 | Sense 5′-3′ | GACCAGAGCCCUCCGGAGACU |
| 29 | Sense 5′-3′ | ACCAGAGCCCUCCGGAGACUA |
| 30 | Sense 5′-3′ | AGGGAAUGGGGAGGAGGUCAU |
| 31 | Sense 5′-3′ | AGGAGGUCAUCAGCUUUGCUA |
| 32 | Sense 5′-3′ | GAGGUCAUCAGCUUUGCUACU |
| 33 | Sense 5′-3′ | GCUUUGCUACUGUCACAGACU |
| 34 | Sense 5′-3′ | CGGUCCCACCACCUGUACCAU |
| 35 | Sense 5′-3′ | GGUCCCACCACCUGUACCAUG |
| 36 | Sense 5′-3′ | GUCCCACCACCUGUACCAUGC |
| 37 | Sense 5′-3′ | UCCCACCACCUGUACCAUGCC |
| 38 | Sense 5′-3′ | CCCACCACCUGUACCAUGCCC |
| 39 | Sense 5′-3′ | CCACCACCUGUACCAUGCCCG |
| 40 | Sense 5′-3′ | CACCACCUGUACCAUGCCCGC |
| 41 | Sense 5′-3′ | ACCACCUGUACCAUGCCCGCC |
| 42 | Sense 5′-3′ | CCACCUGUACCAUGCCCGCCU |
| 43 | Sense 5′-3′ | CACCUGUACCAUGCCCGCCUG |
| 44 | Sense 5′-3′ | CCACCCUUCCUGGCACUCUUU |
| 45 | Sense 5′-3′ | CCCUUCCUGGCACUCUUUGCU |
| 46 | Sense 5′-3′ | UGGCACUCUUUGCUUGAGGAU |
| 47 | Sense 5′-3′ | GCACUCUUUGCUUGAGGAUCU |
| 48 | Sense 5′-3′ | CACUCUUUGCUUGAGGAUCUU |
| 49 | Sense 5′-3′ | CUAGUGGCUUGAGGGGUGAGA |
| 50 | Sense 5′-3′ | GGCUUGAGGGGUGAGAAGUCU |
| 51 | Sense 5′-3′ | GAAGUCUGGUGUCCUGAAACU |
| 52 | Sense 5′-3′ | UGGUGUCCUGAAACUGCAACU |
| 53 | Sense 5′-3′ | GGUGUCCUGAAACUGCAACUA |
| 54 | Sense 5′-3′ | CAGCCCUUCCUAGAGCUUAAG |
| 55 | Sense 5′-3′ | GCCCUUCCUAGAGCUUAAGAU |
| 56 | Sense 5′-3′ | GAGCUUAAGAUCCGAGCCAAU |
| 57 | Sense 5′-3′ | CCAUUACGUAGACUUCCAGGA |
| 58 | Sense 5′-3′ | CCCGAGGGGUACCAGCUGAAU |
| 59 | Sense 5′-3′ | CGAGGGGUACCAGCUGAAUUA |
| 60 | Sense 5′-3′ | GGUACCAGCUGAAUUACUGCA |
| 61 | Sense 5′-3′ | GUACCAGCUGAAUUACUGCAG |
| 62 | Sense 5′-3′ | UACCAGCUGAAUUACUGCAGU |
| 63 | Sense 5′-3′ | ACCAGCUGAAUUACUGCAGUG |
| 64 | Sense 5′-3′ | CCAGCUGAAUUACUGCAGUGG |
| 65 | Sense 5′-3′ | AGCUGAAUUACUGCAGUGGGC |
| 66 | Sense 5′-3′ | GCUGAAUUACUGCAGUGGGCA |
| 67 | Sense 5′-3′ | CUGAAUUACUGCAGUGGGCAG |
| 68 | Sense 5′-3′ | AUUACUGCAGUGGGCAGUGCC |
| 69 | Sense 5′-3′ | GGCAUUGCUGCCUCUUUCCAU |
| 70 | Sense 5′-3′ | GCAUUGCUGCCUCUUUCCAUU |
| 71 | Sense 5′-3′ | CUCUUUCCAUUCUGCCGUCUU |
| 72 | Sense 5′-3′ | UCAGCCUCCUCAAAGCCAACA |
| 73 | Sense 5′-3′ | CAGCCUCCUCAAAGCCAACAA |
| 74 | Sense 5′-3′ | UCCUCAAAGCCAACAAUCCUU |
| 75 | Sense 5′-3′ | UCUCUCCUCUACCUGGAUCAU |
| 76 | Sense 5′-3′ | UCUCCUCUACCUGGAUCAUAA |
| 77 | Sense 5′-3′ | CUCCUCUACCUGGAUCAUAAU |
| 78 | Sense 5′-3′ | UACCUGGAUCAUAAUGGCAAU |
| 79 | Sense 5′-3′ | CCUGGAUCAUAAUGGCAAUGU |
| 80 | Sense 5′-3′ | AUCAUAAUGGCAAUGUGGUCA |
| 81 | Sense 5′-3′ | UCAUAAUGGCAAUGUGGUCAA |
| 82 | Sense 5′-3′ | CAUAAUGGCAAUGUGGUCAAG |
| 83 | Sense 5′-3′ | AUAAUGGCAAUGUGGUCAAGA |
| 84 | Sense 5′-3′ | UAAUGGCAAUGUGGUCAAGAC |
| 85 | Sense 5′-3′ | AAUGGCAAUGUGGUCAAGACG |
| 86 | Sense 5′-3′ | CAAUGUGGUCAAGACGGAUGU |
| 87 | Sense 5′-3′ | CAAGACGGAUGUGCCAGAUAU |
| 88 | Sense 5′-3′ | GAGGCCUGUGGCUGCAGCUAG |
| 89 | Sense 5′-3′ | AGGCCUGUGGCUGCAGCUAGC |
| 90 | Sense 5′-3′ | GGCCUGUGGCUGCAGCUAGCA |
| 91 | Sense 5′-3′ | GCCUGUGGCUGCAGCUAGCAA |
| 92 | Sense 5′-3′ | CCUGUGGCUGCAGCUAGCAAG |
| 93 | Sense 5′-3′ | GCUUUGGAGUGAAGAGACCAA |
| 94 | Sense 5′-3′ | CAACCACCUGGCAAUAUGACU |
| 95 | Sense 5′-3′ | ACCACCUGGCAAUAUGACUCA |
| 96 | Sense 5′-3′ | CACCUGGCAAUAUGACUCACU |
| 97 | Sense 5′-3′ | GGACCCAAAUGGGCACUUUCU |
| 98 | Sense 5′-3′ | CCCAAAUGGGCACUUUCUUGU |
| 99 | Sense 5′-3′ | CAAAUGGGCACUUUCUUGUCU |
| 100 | Sense 5′-3′ | GGCACUUUCUUGUCUGAGACU |
| 101 | Sense 5′-3′ | CACUUUCUUGUCUGAGACUCU |
| 102 | Sense 5′-3′ | CUUGUCUGAGACUCUGGCUUA |
| 103 | Sense 5′-3′ | AGGGAAGGCAGAGAAAAAUUA |
| 104 | Sense 5′-3′ | GGGAAGGCAGAGAAAAAUUAC |
| 105 | Sense 5′-3′ | AGCCUCUCCCAAGAUGAGAAA |
| 106 | Sense 5′-3′ | CCUCUCCCAAGAUGAGAAAGU |
| 107 | Sense 5′-3′ | CUCCCAAGAUGAGAAAGUCCU |
| 108 | Sense 5′-3′ | GGGGAGGAGGAAGCAGAUAGA |
| 109 | Sense 5′-3′ | GGGAGGAGGAAGCAGAUAGAU |
| 110 | Sense 5′-3′ | CAGAAACAGGAGUCAGGAAAA |
| 111 | Sense 5′-3′ | GCACUAAGCCUAAGAAGUUCC |
| 112 | Sense 5′-3′ | CCCACUGGGAGACAAGCAUUU |
| 113 | Sense 5′-3′ | CCACUGGGAGACAAGCAUUUA |
| 114 | Sense 5′-3′ | ACUGGGAGACAAGCAUUUAUA |
| 115 | Sense 5′-3′ | UGGGAGACAAGCAUUUAUACU |
| 116 | Sense 5′-3′ | GGGAGACAAGCAUUUAUACUU |
| 117 | Sense 5′-3′ | GACAAGCAUUUAUACUUUCUU |
| 118 | Sense 5′-3′ | GAGCCACCGCGCCUGGCUUAU |
| 119 | Sense 5′-3′ | CCACCGCGCCUGGCUUAUACU |
| 120 | Sense 5′-3′ | ACCGCGCCUGGCUUAUACUUU |
| 121 | Sense 5′-3′ | CGCGCCUGGCUUAUACUUUCU |
| 122 | Sense 5′-3′ | GCGCCUGGCUUAUACUUUCUU |
| 123 | Sense 5′-3′ | CGCCUGGCUUAUACUUUCUUA |
| 124 | Sense 5′-3′ | GCCUGGCUUAUACUUUCUUAA |
| 125 | Sense 5′-3′ | CCUGGCUUAUACUUUCUUAAU |
| 126 | Sense 5′-3′ | UGGCUUAUACUUUCUUAAUAA |
| 127 | Sense 5′-3′ | GGCUUAUACUUUCUUAAUAAA |
| 128 | Sense 5′-3′ | CUUAUACUUUCUUAAUAAAAA |
| 129 | Sense 5′-3′ | AGGGGUGUCCACAAAGUCAAA |
| 130 | Sense 5′-3′ | GGGGUGUCCACAAAGUCAAAG |
| 131 | Sense 5′-3′ | UCAUAAUAAUACUAACAUGUU |
| 132 | Sense 5′-3′ | ACUAACAUGUUAUUUGCCUUU |
| 133 | Sense 5′-3′ | GUUAUUUGCCUUUUGAAUUCU |
| 134 | Sense 5′-3′ | UGCCUUUUGAAUUCUCAUUAU |
| 135 | Sense 5′-3′ | GCCUUUUGAAUUCUCAUUAUC |
| 136 | Sense 5′-3′ | CCUUUUGAAUUCUCAUUAUCU |
| 137 | Sense 5′-3′ | CUUUUGAAUUCUCAUUAUCUU |
| 138 | Sense 5′-3′ | UGAAUUCUCAUUAUCUUAAAA |
| 139 | Sense 5′-3′ | GAAUUCUCAUUAUCUUAAAAU |
| 140 | Sense 5′-3′ | CCGUGUGACAUGUGAUUACAU |
| 141 | Sense 5′-3′ | UGUGACAUGUGAUUACAUCAU |
| 142 | Sense 5′-3′ | UGACAUGUGAUUACAUCAUCU |
| 143 | Sense 5′-3′ | GACAUGUGAUUACAUCAUCUU |
| 144 | Sense 5′-3′ | UCUUUCUGACAUCAUUGUUAA |
| 145 | Sense 5′-3′ | CUUUCUGACAUCAUUGUUAAU |
| 146 | Sense 5′-3′ | GACAUCAUUGUUAAUGGAAUG |
| 147 | Sense 5′-3′ | GUUAAUGGAAUGUGUGCUUGU |
| 147 | Antisense 5′-3′ | AUGGAUAGCUGUGCUUGACCCUC |
| 148 | Antisense 5′-3′ | AUCAUCUGAUGGAUAGCUGUGCU |
| 149 | Antisense 5′-3′ | AGAUCAUCUGAUGGAUAGCUGUG |
| 150 | Antisense 5′-3′ | UAGAUCAUCUGAUGGAUAGCUGU |
| 151 | Antisense 5′-3′ | AGUAGAUCAUCUGAUGGAUAGCU |
| 152 | Antisense 5′-3′ | AAGUAGAUCAUCUGAUGGAUAGC |
| 153 | Antisense 5′-3′ | UGAAAGUAGAUCAUCUGAUGGAU |
| 154 | Antisense 5′-3′ | ACUCAGGAAGGCUGAAAGUAGAU |
| 155 | Antisense 5′-3′ | AGCCACCUGUCUUCUAUUGUCUG |
| 156 | Antisense 5′-3′ | AGUGACAGCAGACACCACUGCCA |
| 157 | Antisense 5′-3′ | UGAUUGCUGGGGGCCAAUGAGGG |
| 158 | Antisense 5′-3′ | AGUUUGGAGCCCCCACAGGAGGG |
| 159 | Antisense 5′-3′ | UUGCUGCUUGGCUAGCUCCAGCA |
| 160 | Antisense 5′-3′ | UUUGCUGCUUGGCUAGCUCCAGC |
| 161 | Antisense 5′-3′ | AUUUGCUGCUUGGCUAGCUCCAG |
| 162 | Antisense 5′-3′ | GAUUUGCUGCUUGGCUAGCUCCA |
| 163 | Antisense 5′-3′ | GGAUUUGCUGCUUGGCUAGCUCC |
| 164 | Antisense 5′-3′ | AGGAUUUGCUGCUUGGCUAGCUC |
| 165 | Antisense 5′-3′ | CCAGGAUUUGCUGCUUGGCUAGC |
| 166 | Antisense 5′-3′ | AUCCAGGAUUUGCUGCUUGGCUA |
| 167 | Antisense 5′-3′ | UUAUUCUGGGACGACUGGUCAGG |
| 168 | Antisense 5′-3′ | AGUUAUUCUGGGACGACUGGUCA |
| 169 | Antisense 5′-3′ | AGGAUGAGUUAUUCUGGGACGAC |
| 170 | Antisense 5′-3′ | UCCGGAGGGCUCUGGUCAGCGCU |
| 171 | Antisense 5′-3′ | CUCCGGAGGGCUCUGGUCAGCGC |
| 172 | Antisense 5′-3′ | UCUCCGGAGGGCUCUGGUCAGCG |
| 173 | Antisense 5′-3′ | GUCUCCGGAGGGCUCUGGUCAGC |
| 174 | Antisense 5′-3′ | AGUCUCCGGAGGGCUCUGGUCAG |
| 175 | Antisense 5′-3′ | UAGUCUCCGGAGGGCUCUGGUCA |
| 176 | Antisense 5′-3′ | AUGACCUCCUCCCCAUUCCCUGG |
| 177 | Antisense 5′-3′ | UAGCAAAGCUGAUGACCUCCUCC |
| 178 | Antisense 5′-3′ | AGUAGCAAAGCUGAUGACCUCCU |
| 179 | Antisense 5′-3′ | AGUCUGUGACAGUAGCAAAGCUG |
| 180 | Antisense 5′-3′ | AUGGUACAGGUGGUGGGACCGAG |
| 181 | Antisense 5′-3′ | CAUGGUACAGGUGGUGGGACCGA |
| 182 | Antisense 5′-3′ | GCAUGGUACAGGUGGUGGGACCG |
| 183 | Antisense 5′-3′ | GGCAUGGUACAGGUGGUGGGACC |
| 184 | Antisense 5′-3′ | GGGCAUGGUACAGGUGGUGGGAC |
| 185 | Antisense 5′-3′ | CGGGCAUGGUACAGGUGGUGGGA |
| 186 | Antisense 5′-3′ | GCGGGCAUGGUACAGGUGGUGGG |
| 187 | Antisense 5′-3′ | GGCGGGCAUGGUACAGGUGGUGG |
| 188 | Antisense 5′-3′ | AGGCGGGCAUGGUACAGGUGGUG |
| 189 | Antisense 5′-3′ | CAGGCGGGCAUGGUACAGGUGGU |
| 190 | Antisense 5′-3′ | AAAGAGUGCCAGGAAGGGUGGGG |
| 191 | Antisense 5′-3′ | AGCAAAGAGUGCCAGGAAGGGUG |
| 192 | Antisense 5′-3′ | AUCCUCAAGCAAAGAGUGCCAGG |
| 193 | Antisense 5′-3′ | AGAUCCUCAAGCAAAGAGUGCCA |
| 194 | Antisense 5′-3′ | AAGAUCCUCAAGCAAAGAGUGCC |
| 195 | Antisense 5′-3′ | UCUCACCCCUCAAGCCACUAGAG |
| 196 | Antisense 5′-3′ | AGACUUCUCACCCCUCAAGCCAC |
| 197 | Antisense 5′-3′ | AGUUUCAGGACACCAGACUUCUC |
| 198 | Antisense 5′-3′ | AGUUGCAGUUUCAGGACACCAGA |
| 199 | Antisense 5′-3′ | UAGUUGCAGUUUCAGGACACCAG |
| 200 | Antisense 5′-3′ | CUUAAGCUCUAGGAAGGGCUGCU |
| 201 | Antisense 5′-3′ | AUCUUAAGCUCUAGGAAGGGCUG |
| 202 | Antisense 5′-3′ | AUUGGCUCGGAUCUUAAGCUCUA |
| 203 | Antisense 5′-3′ | UCCUGGAAGUCUACGUAAUGGUC |
| 204 | Antisense 5′-3′ | AUUCAGCUGGUACCCCUCGGGCU |
| 205 | Antisense 5′-3′ | UAAUUCAGCUGGUACCCCUCGGG |
| 206 | Antisense 5′-3′ | UGCAGUAAUUCAGCUGGUACCCC |
| 207 | Antisense 5′-3′ | CUGCAGUAAUUCAGCUGGUACCC |
| 208 | Antisense 5′-3′ | ACUGCAGUAAUUCAGCUGGUACC |
| 209 | Antisense 5′-3′ | CACUGCAGUAAUUCAGCUGGUAC |
| 210 | Antisense 5′-3′ | CCACUGCAGUAAUUCAGCUGGUA |
| 211 | Antisense 5′-3′ | GCCCACUGCAGUAAUUCAGCUGG |
| 212 | Antisense 5′-3′ | UGCCCACUGCAGUAAUUCAGCUG |
| 213 | Antisense 5′-3′ | CUGCCCACUGCAGUAAUUCAGCU |
| 214 | Antisense 5′-3′ | GGCACUGCCCACUGCAGUAAUUC |
| 215 | Antisense 5′-3′ | AUGGAAAGAGGCAGCAAUGCCUG |
| 216 | Antisense 5′-3′ | AAUGGAAAGAGGCAGCAAUGCCU |
| 217 | Antisense 5′-3′ | AAGACGGCAGAAUGGAAAGAGGC |
| 218 | Antisense 5′-3′ | UGUUGGCUUUGAGGAGGCUGAAG |
| 219 | Antisense 5′-3′ | UUGUUGGCUUUGAGGAGGCUGAA |
| 220 | Antisense 5′-3′ | AAGGAUUGUUGGCUUUGAGGAGG |
| 221 | Antisense 5′-3′ | AUGAUCCAGGUAGAGGAGAGAGA |
| 222 | Antisense 5′-3′ | UUAUGAUCCAGGUAGAGGAGAGA |
| 223 | Antisense 5′-3′ | AUUAUGAUCCAGGUAGAGGAGAG |
| 224 | Antisense 5′-3′ | AUUGCCAUUAUGAUCCAGGUAGA |
| 225 | Antisense 5′-3′ | ACAUUGCCAUUAUGAUCCAGGUA |
| 226 | Antisense 5′-3′ | UGACCACAUUGCCAUUAUGAUCC |
| 227 | Antisense 5′-3′ | UUGACCACAUUGCCAUUAUGAUC |
| 228 | Antisense 5′-3′ | CUUGACCACAUUGCCAUUAUGAU |
| 229 | Antisense 5′-3′ | UCUUGACCACAUUGCCAUUAUGA |
| 230 | Antisense 5′-3′ | GUCUUGACCACAUUGCCAUUAUG |
| 231 | Antisense 5′-3′ | CGUCUUGACCACAUUGCCAUUAU |
| 232 | Antisense 5′-3′ | ACAUCCGUCUUGACCACAUUGCC |
| 233 | Antisense 5′-3′ | AUAUCUGGCACAUCCGUCUUGAC |
| 234 | Antisense 5′-3′ | CUAGCUGCAGCCACAGGCCUCCA |
| 235 | Antisense 5′-3′ | GCUAGCUGCAGCCACAGGCCUCC |
| 236 | Antisense 5′-3′ | UGCUAGCUGCAGCCACAGGCCUC |
| 237 | Antisense 5′-3′ | UUGCUAGCUGCAGCCACAGGCCU |
| 238 | Antisense 5′-3′ | CUUGCUAGCUGCAGCCACAGGCC |
| 239 | Antisense 5′-3′ | UUGGUCUCUUCACUCCAAAGCCC |
| 240 | Antisense 5′-3′ | AGUCAUAUUGCCAGGUGGUUGUU |
| 241 | Antisense 5′-3′ | UGAGUCAUAUUGCCAGGUGGUUG |
| 242 | Antisense 5′-3′ | AGUGAGUCAUAUUGCCAGGUGGU |
| 243 | Antisense 5′-3′ | AGAAAGUGCCCAUUUGGGUCCCA |
| 244 | Antisense 5′-3′ | ACAAGAAAGUGCCCAUUUGGGUC |
| 245 | Antisense 5′-3′ | AGACAAGAAAGUGCCCAUUUGGG |
| 246 | Antisense 5′-3′ | AGUCUCAGACAAGAAAGUGCCCA |
| 247 | Antisense 5′-3′ | AGAGUCUCAGACAAGAAAGUGCC |
| 248 | Antisense 5′-3′ | UAAGCCAGAGUCUCAGACAAGAA |
| 249 | Antisense 5′-3′ | UAAUUUUUCUCUGCCUUCCCUCC |
| 250 | Antisense 5′-3′ | GUAAUUUUUCUCUGCCUUCCCUC |
| 251 | Antisense 5′-3′ | UUUCUCAUCUUGGGAGAGGCUAA |
| 252 | Antisense 5′-3′ | ACUUUCUCAUCUUGGGAGAGGCU |
| 253 | Antisense 5′-3′ | AGGACUUUCUCAUCUUGGGAGAG |
| 254 | Antisense 5′-3′ | UCUAUCUGCUUCCUCCUCCCCUC |
| 255 | Antisense 5′-3′ | AUCUAUCUGCUUCCUCCUCCCCU |
| 256 | Antisense 5′-3′ | UUUUCCUGACUCCUGUUUCUGGG |
| 257 | Antisense 5′-3′ | GGAACUUCUUAGGCUUAGUGCCU |
| 258 | Antisense 5′-3′ | AAAUGCUUGUCUCCCAGUGGGUC |
| 259 | Antisense 5′-3′ | UAAAUGCUUGUCUCCCAGUGGGU |
| 260 | Antisense 5′-3′ | UAUAAAUGCUUGUCUCCCAGUGG |
| 261 | Antisense 5′-3′ | AGUAUAAAUGCUUGUCUCCCAGU |
| 262 | Antisense 5′-3′ | AAGUAUAAAUGCUUGUCUCCCAG |
| 263 | Antisense 5′-3′ | AAGAAAGUAUAAAUGCUUGUCUC |
| 264 | Antisense 5′-3′ | AUAAGCCAGGCGCGGUGGCUCAC |
| 265 | Antisense 5′-3′ | AGUAUAAGCCAGGCGCGGUGGCU |
| 266 | Antisense 5′-3′ | AAAGUAUAAGCCAGGCGCGGUGG |
| 267 | Antisense 5′-3′ | AGAAAGUAUAAGCCAGGCGCGGU |
| 268 | Antisense 5′-3′ | AAGAAAGUAUAAGCCAGGCGCGG |
| 269 | Antisense 5′-3′ | UAAGAAAGUAUAAGCCAGGCGCG |
| 270 | Antisense 5′-3′ | UUAAGAAAGUAUAAGCCAGGCGC |
| 271 | Antisense 5′-3′ | AUUAAGAAAGUAUAAGCCAGGCG |
| 272 | Antisense 5′-3′ | UUAUUAAGAAAGUAUAAGCCAGG |
| 273 | Antisense 5′-3′ | UUUAUUAAGAAAGUAUAAGCCAG |
| 274 | Antisense 5′-3′ | UUUUUAUUAAGAAAGUAUAAGCC |
| 275 | Antisense 5′-3′ | UUUGACUUUGUGGACACCCCUGA |
| 276 | Antisense 5′-3′ | CUUUGACUUUGUGGACACCCCUG |
| 277 | Antisense 5′-3′ | AACAUGUUAGUAUUAUUAUGAAA |
| 278 | Antisense 5′-3′ | AAAGGCAAAUAACAUGUUAGUAU |
| 279 | Antisense 5′-3′ | AGAAUUCAAAAGGCAAAUAACAU |
| 280 | Antisense 5′-3′ | AUAAUGAGAAUUCAAAAGGCAAA |
| 281 | Antisense 5′-3′ | GAUAAUGAGAAUUCAAAAGGCAA |
| 282 | Antisense 5′-3′ | AGAUAAUGAGAAUUCAAAAGGCA |
| 283 | Antisense 5′-3′ | AAGAUAAUGAGAAUUCAAAAGGC |
| 284 | Antisense 5′-3′ | UUUUAAGAUAAUGAGAAUUCAAA |
| 285 | Antisense 5′-3′ | AUUUUAAGAUAAUGAGAAUUCAA |
| 286 | Antisense 5′-3′ | AUGUAAUCACAUGUCACACGGCC |
| 287 | Antisense 5′-3′ | AUGAUGUAAUCACAUGUCACACG |
| 288 | Antisense 5′-3′ | AGAUGAUGUAAUCACAUGUCACA |
| 289 | Antisense 5′-3′ | AAGAUGAUGUAAUCACAUGUCAC |
| 290 | Antisense 5′-3′ | UUAACAAUGAUGUCAGAAAGAUG |
| 291 | Antisense 5′-3′ | AUUAACAAUGAUGUCAGAAAGAU |
| 292 | Antisense 5′-3′ | CAUUCCAUUAACAAUGAUGUCAG |
| 293 | Antisense 5′-3′ | ACAAGCACACAUUCCAUUAACAA |
| 294 | Sense 5′-3′ | g*g*gucaAgCACagcuauccau |
| | (modified) | |
| 295 | Sense 5′-3′ | c*a*cagcUaUCCaucagaugau |
| | (modified) | |
| 296 | Sense 5′-3′ | c*a*gcuaUcCAUcagaugaucu |
| | (modified) | |
| 297 | Sense 5′-3′ | a*g*cuauCcAUCagaugaucua |
| | (modified) | |
| 298 | Sense 5′-3′ | c*u*auccAuCAGaugaucuacu |
| | (modified) | |
| 299 | Sense 5′-3′ | u*a*uccaUcAGAugaucuacuu |
| | (modified) | |
| 300 | Sense 5′-3′ | c*c*aucaGaUGAucuacuuuca |
| | (modified) | |
| 301 | Sense 5′-3′ | c*u*acuuUcAGCcuuccugagu |
| | (modified) | |
| 302 | Sense 5′-3′ | g*a*caauAgAAGacagguggcu |
| | (modified) | |
| 303 | Sense 5′-3′ | g*c*agugGuGUCugcugucacu |
| | (modified) | |
| 304 | Sense 5′-3′ | c*u*cauuGgCCCccagcaauca |
| | (modified) | |
| 305 | Sense 5′-3′ | c*u*ccugUgGGGgcuccaaacu |
| | (modified) | |
| 306 | Sense 5′-3′ | c*u*ggagCuAGCcaagcagcaa |
| | (modified) | |
| 307 | Sense 5′-3′ | u*g*gagcUaGCCaagcagcaaa |
| | (modified) | |
| 308 | Sense 5′-3′ | g*g*agcuAgCCAagcagcaaau |
| | (modified) | |
| 309 | Sense 5′-3′ | g*a*gcuaGcCAAgcagcaaauc |
| | (modified) | |
| 310 | Sense 5′-3′ | a*g*cuagCcAAGcagcaaaucc |
| | (modified) | |
| 311 | Sense 5′-3′ | g*c*uagcCaAGCagcaaauccu |
| | (modified) | |
| 312 | Sense 5′-3′ | u*a*gccaAgCAGcaaauccugg |
| | (modified) | |
| 313 | Sense 5′-3′ | g*c*caagCaGCAaauccuggau |
| | (modified) | |
| 314 | Sense 5′-3′ | u*g*accaGuCGUcccagaauaa |
| | (modified) | |
| 315 | Sense 5′-3′ | a*c*caguCgUCCcagaauaacu |
| | (modified) | |
| 316 | Sense 5′-3′ | c*g*ucccAgAAUaacucauccu |
| | (modified) | |
| 317 | Sense 5′-3′ | c*g*cugaCcAGAgcccuccgga |
| | (modified) | |
| 318 | Sense 5′-3′ | g*c*ugacCaGAGcccuccggag |
| | (modified) | |
| 319 | Sense 5′-3′ | c*u*gaccAgAGCccuccggaga |
| | (modified) | |
| 320 | Sense 5′-3′ | u*g*accaGaGCCcuccggagac |
| | (modified) | |
| 321 | Sense 5′-3′ | g*a*ccagAgCCCuccggagacu |
| | (modified) | |
| 322 | Sense 5′-3′ | a*c*cagaGcCCUccggagacua |
| | (modified) | |
| 323 | Sense 5′-3′ | a*g*ggaaUgGGGaggaggucau |
| | (modified) | |
| 324 | Sense 5′-3′ | a*g*gaggUcAUCagcuuugcua |
| | (modified) | |
| 325 | Sense 5′-3′ | g*a*ggucAuCAGcuuugcuacu |
| | (modified) | |
| 326 | Sense 5′-3′ | g*c*uuugCuACUgucacagacu |
| | (modified) | |
| 327 | Sense 5′-3′ | c*g*guccCaCCAccuguaccau |
| | (modified) | |
| 328 | Sense 5′-3′ | g*g*ucccAcCACcuguaccaug |
| | (modified) | |
| 329 | Sense 5′-3′ | g*u*cccaCcACCuguaccaugc |
| | (modified) | |
| 330 | Sense 5′-3′ | u*c*ccacCaCCUguaccaugcc |
| | (modified) | |
| 331 | Sense 5′-3′ | c*c*caccAcCUGuaccaugccc |
| | (modified) | |
| 332 | Sense 5′-3′ | c*c*accaCcUGUaccaugcccg |
| | (modified) | |
| 333 | Sense 5′-3′ | c*a*ccacCuGUAccaugcccgc |
| | (modified) | |
| 334 | Sense 5′-3′ | a*c*caccUgUACcaugcccgcc |
| | (modified) | |
| 335 | Sense 5′-3′ | c*c*accuGuACCaugcccgccu |
| | (modified) | |
| 336 | Sense 5′-3′ | c*a*ccugUaCCAugcccgccug |
| | (modified) | |
| 337 | Sense 5′-3′ | c*c*acccUuCCUggcacucuuu |
| | (modified) | |
| 338 | Sense 5′-3′ | c*c*cuucCuGGCacucuuugcu |
| | (modified) | |
| 339 | Sense 5′-3′ | u*g*gcacUcUUUgcuugaggau |
| | (modified) | |
| 340 | Sense 5′-3′ | g*c*acucUuUGCuugaggaucu |
| | (modified) | |
| 341 | Sense 5′-3′ | c*a*cucuUuGCUugaggaucuu |
| | (modified) | |
| 342 | Sense 5′-3′ | c*u*agugGcUUGaggggugaga |
| | (modified) | |
| 343 | Sense 5′-3′ | g*g*cuugAgGGGugagaagucu |
| | (modified) | |
| 344 | Sense 5′-3′ | g*a*agucUgGUGuccugaaacu |
| | (modified) | |
| 345 | Sense 5′-3′ | u*g*guguCcUGAaacugcaacu |
| | (modified) | |
| 346 | Sense 5′-3′ | g*g*ugucCuGAAacugcaacua |
| | (modified) | |
| 347 | Sense 5′-3′ | c*a*gcccUuCCUagagcuuaag |
| | (modified) | |
| 348 | Sense 5′-3′ | g*c*ccuuCcUAGagcuuaagau |
| | (modified) | |
| 349 | Sense 5′-3′ | g*a*gcuuAaGAUccgagccaau |
| | (modified) | |
| 350 | Sense 5′-3′ | c*c*auuaCgUAGacuuccagga |
| | (modified) | |
| 351 | Sense 5′-3′ | c*c*cgagGgGUAccagcugaau |
| | (modified) | |
| 352 | Sense 5′-3′ | c*g*agggGuACCagcugaauua |
| | (modified) | |
| 353 | Sense 5′-3′ | g*g*uaccAgCUGaauuacugca |
| | (modified) | |
| 354 | Sense 5′-3′ | g*u*accaGcUGAauuacugcag |
| | (modified) | |
| 355 | Sense 5′-3′ | u*a*ccagCuGAAuuacugcagu |
| | (modified) | |
| 356 | Sense 5′-3′ | a*c*cagcUgAAUuacugcagug |
| | (modified) | |
| 357 | Sense 5′-3′ | c*c*agcuGaAUUacugcagugg |
| | (modified) | |
| 358 | Sense 5′-3′ | a*g*cugaAuUACugcagugggc |
| | (modified) | |
| 359 | Sense 5′-3′ | g*c*ugaaUuACUgcagugggca |
| | (modified) | |
| 360 | Sense 5′-3′ | c*u*gaauUaCUGcagugggcag |
| | (modified) | |
| 361 | Sense 5′-3′ | a*u*uacuGcAGUgggcagugcc |
| | (modified) | |
| 362 | Sense 5′-3′ | g*g*cauuGcUGCcucuuuccau |
| | (modified) | |
| 363 | Sense 5′-3′ | g*c*auugCuGCCucuuuccauu |
| | (modified) | |
| 364 | Sense 5′-3′ | c*u*cuuuCcAUUcugccgucuu |
| | (modified) | |
| 365 | Sense 5′-3′ | u*c*agccUcCUCaaagccaaca |
| | (modified) | |
| 366 | Sense 5′-3′ | c*a*gccuCcUCAaagccaacaa |
| | (modified) | |
| 367 | Sense 5′-3′ | u*c*cucaAaGCCaacaauccuu |
| | (modified) | |
| 368 | Sense 5′-3′ | u*c*ucucCuCUAccuggaucau |
| | (modified) | |
| 369 | Sense 5′-3′ | u*c*uccuCuACCuggaucauaa |
| | (modified) | |
| 370 | Sense 5′-3′ | c*u*ccucUaCCUggaucauaau |
| | (modified) | |
| 371 | Sense 5′-3′ | u*a*ccugGaUCAuaauggcaau |
| | (modified) | |
| 372 | Sense 5′-3′ | c*c*uggaUcAUAauggcaaugu |
| | (modified) | |
| 373 | Sense 5′-3′ | a*u*cauaAuGGCaaugugguca |
| | (modified) | |
| 374 | Sense 5′-3′ | u*c*auaaUgGCAauguggucaa |
| | (modified) | |
| 375 | Sense 5′-3′ | c*a*uaauGgCAAuguggucaag |
| | (modified) | |
| 376 | Sense 5′-3′ | a*u*aaugGcAAUguggucaaga |
| | (modified) | |
| 377 | Sense 5′-3′ | u*a*auggCaAUGuggucaagac |
| | (modified) | |
| 378 | Sense 5′-3′ | a*a*uggcAaUGUggucaagacg |
| | (modified) | |
| 379 | Sense 5′-3′ | c*a*auguGgUCAagacggaugu |
| | (modified) | |
| 380 | Sense 5′-3′ | c*a*agacGgAUGugccagauau |
| | (modified) | |
| 381 | Sense 5′-3′ | g*a*ggccUgUGGcugcagcuag |
| | (modified) | |
| 382 | Sense 5′-3′ | a*g*gccuGuGGCugcagcuagc |
| | (modified) | |
| 383 | Sense 5′-3′ | g*g*ccugUgGCUgcagcuagca |
| | (modified) | |
| 384 | Sense 5′-3′ | g*c*cuguGgCUGcagcuagcaa |
| | (modified) | |
| 385 | Sense 5′-3′ | c*c*ugugGcUGCagcuagcaag |
| | (modified) | |
| 386 | Sense 5′-3′ | g*c*uuugGaGUGaagagaccaa |
| | (modified) | |
| 387 | Sense 5′-3′ | c*a*accaCcUGGcaauaugacu |
| | (modified) | |
| 388 | Sense 5′-3′ | a*c*caccUgGCAauaugacuca |
| | (modified) | |
| 389 | Sense 5′-3′ | c*a*ccugGcAAUaugacucacu |
| | (modified) | |
| 390 | Sense 5′-3′ | g*g*acccAaAUGggcacuuucu |
| | (modified) | |
| 391 | Sense 5′-3′ | c*c*caaaUgGGCacuuucuugu |
| | (modified) | |
| 392 | Sense 5′-3′ | c*a*aaugGgCACuuucuugucu |
| | (modified) | |
| 393 | Sense 5′-3′ | g*g*cacuUuCUUgucugagacu |
| | (modified) | |
| 394 | Sense 5′-3′ | c*a*cuuuCuUGUcugagacucu |
| | (modified) | |
| 395 | Sense 5′-3′ | c*u*ugucUgAGAcucuggcuua |
| | (modified) | |
| 396 | Sense 5′-3′ | a*g*ggaaGgCAGagaaaaauua |
| | (modified) | |
| 397 | Sense 5′-3′ | g*g*gaagGcAGAgaaaaauuac |
| | (modified) | |
| 398 | Sense 5′-3′ | a*g*ccucUcCCAagaugagaaa |
| | (modified) | |
| 399 | Sense 5′-3′ | c*c*ucucCcAAGaugagaaagu |
| | (modified) | |
| 400 | Sense 5′-3′ | c*u*cccaAgAUGagaaaguccu |
| | (modified) | |
| 401 | Sense 5′-3′ | g*g*ggagGaGGAagcagauaga |
| | (modified) | |
| 402 | Sense 5′-3′ | g*g*gaggAgGAAgcagauagau |
| | (modified) | |
| 403 | Sense 5′-3′ | c*a*gaaaCaGGAgucaggaaaa |
| | (modified) | |
| 404 | Sense 5′-3′ | g*c*acuaAgCCUaagaaguucc |
| | (modified) | |
| 405 | Sense 5′-3′ | c*c*cacuGgGAGacaagcauuu |
| | (modified) | |
| 406 | Sense 5′-3′ | c*c*acugGgAGAcaagcauuua |
| | (modified) | |
| 407 | Sense 5′-3′ | a*c*ugggAgACAagcauuuaua |
| | (modified) | |
| 408 | Sense 5′-3′ | u*g*ggagAcAAGcauuuauacu |
| | (modified) | |
| 409 | Sense 5′-3′ | g*g*gagaCaAGCauuuauacuu |
| | (modified) | |
| 410 | Sense 5′-3′ | g*a*caagCaUUUauacuuucuu |
| | (modified) | |
| 411 | Sense 5′-3′ | g*a*gccaCcGCGccuggcuuau |
| | (modified) | |
| 412 | Sense 5′-3′ | c*c*accgCgCCUggcuuauacu |
| | (modified) | |
| 413 | Sense 5′-3′ | a*c*cgcgCcUGGcuuauacuuu |
| | (modified) | |
| 414 | Sense 5′-3′ | c*g*cgccUgGCUuauacuuucu |
| | (modified) | |
| 415 | Sense 5′-3′ | g*c*gccuGgCUUauacuuucuu |
| | (modified) | |
| 416 | Sense 5′-3′ | c*g*ccugGcUUAuacuuucuua |
| | (modified) | |
| 417 | Sense 5′-3′ | g*c*cuggCuUAUacuuucuuaa |
| | (modified) | |
| 418 | Sense 5′-3′ | c*c*uggcUuAUAcuuucuuaau |
| | (modified) | |
| 419 | Sense 5′-3′ | u*g*gcuuAuACUuucuuaauaa |
| | (modified) | |
| 420 | Sense 5′-3′ | g*g*cuuaUaCUUucuuaauaaa |
| | (modified) | |
| 421 | Sense 5′-3′ | c*u*uauaCuUUCuuaauaaaaa |
| | (modified) | |
| 422 | Sense 5′-3′ | a*g*ggguGuCCAcaaagucaaa |
| | (modified) | |
| 423 | Sense 5′-3′ | g*g*ggugUcCACaaagucaaag |
| | (modified) | |
| 424 | Sense 5′-3′ | u*c*auaaUaAUAcuaacauguu |
| | (modified) | |
| 425 | Sense 5′-3′ | a*c*uaacAuGUUauuugccuuu |
| | (modified) | |
| 426 | Sense 5′-3′ | g*u*uauuUgCCUuuugaauucu |
| | (modified) | |
| 427 | Sense 5′-3′ | u*g*ccuuUuGAAuucucauuau |
| | (modified) | |
| 428 | Sense 5′-3′ | g*c*cuuuUgAAUucucauuauc |
| | (modified) | |
| 429 | Sense 5′-3′ | c*c*uuuuGaAUUcucauuaucu |
| | (modified) | |
| 430 | Sense 5′-3′ | c*u*uuugAaUUCucauuaucuu |
| | (modified) | |
| 431 | Sense 5′-3′ | u*g*aauuCuCAUuaucuuaaaa |
| | (modified) | |
| 432 | Sense 5′-3′ | g*a*auucUcAUUaucuuaaaau |
| | (modified) | |
| 433 | Sense 5′-3′ | c*c*guguGaCAUgugauuacau |
| | (modified) | |
| 434 | Sense 5′-3′ | u*g*ugacAuGUGauuacaucau |
| | (modified) | |
| 435 | Sense 5′-3′ | u*g*acauGuGAUuacaucaucu |
| | (modified) | |
| 436 | Sense 5′-3′ | g*a*caugUgAUUacaucaucuu |
| | (modified) | |
| 437 | Sense 5′-3′ | u*c*uuucUgACAucauuguuaa |
| | (modified) | |
| 438 | Sense 5′-3′ | c*u*uucuGaCAUcauuguuaau |
| | (modified) | |
| 439 | Sense 5′-3′ | g*a*caucAuUGUuaauggaaug |
| | (modified) | |
| 440 | Sense 5′-3′ | g*u*uaauGgAAUgugugcuugu |
| | (modified) | |
| 441 | Antisense 5′-3′ | a*U*ggaUaGCugugCuUgaccc*u*c |
| | (modified) | |
| 442 | Antisense 5′-3′ | a*U*cauCuGAuggaUaGcugug*c*u |
| | (modified) | |
| 443 | Antisense 5′-3′ | a*G*aucAuCUgaugGaUagcug*u*g |
| | (modified) | |
| 444 | Antisense 5′-3′ | u*A*gauCaUCugauGgAuagcu*g*u |
| | (modified) | |
| 445 | Antisense 5′-3′ | a*G*uagAuCAucugAuGgauag*c*u |
| | (modified) | |
| 446 | Antisense 5′-3′ | a*A*guaGaUCaucuGaUggaua*g*c |
| | (modified) | |
| 447 | Antisense 5′-3′ | u*G*aaaGuAGaucaUcUgaugg*a*u |
| | (modified) | |
| 448 | Antisense 5′-3′ | a*C*ucaGgAAggcuGaAaguag*a*u |
| | (modified) | |
| 449 | Antisense 5′-3′ | a*G*ccaCcUGucuuCuAuuguc*u*g |
| | (modified) | |
| 450 | Antisense 5′-3′ | a*G*ugaCaGCagacAcCacugc*c*a |
| | (modified) | |
| 451 | Antisense 5′-3′ | u*G*auuGcUGggggCcAaugag*g*g |
| | (modified) | |
| 452 | Antisense 5′-3′ | a*G*uuuGgAGccccCaCaggag*g*g |
| | (modified) | |
| 453 | Antisense 5′-3′ | u*U*gcuGcUUggcuAgCuccag*c*a |
| | (modified) | |
| 454 | Antisense 5′-3′ | u*U*ugcUgCUuggcUaGcucca*g*c |
| | (modified) | |
| 455 | Antisense 5′-3′ | a*U*uugCuGCuuggCuAgcucc*a*g |
| | (modified) | |
| 456 | Antisense 5′-3′ | g*A*uuuGcUGcuugGcUagcuc*c*a |
| | (modified) | |
| 457 | Antisense 5′-3′ | g*G*auuUgCUgcuuGgCuagcu*c*c |
| | (modified) | |
| 458 | Antisense 5′-3′ | a*G*gauUuGCugcuUgGcuagc*u*c |
| | (modified) | |
| 459 | Antisense 5′-3′ | c*C*aggAuUUgcugCuUggcua*g*c |
| | (modified) | |
| 460 | Antisense 5′-3′ | a*U*ccaGgAUuugcUgCuuggc*u*a |
| | (modified) | |
| 461 | Antisense 5′-3′ | u*U*auuCuGGgacgAcUgguca*g*g |
| | (modified) | |
| 462 | Antisense 5′-3′ | a*G*uuaUuCUgggaCgAcuggu*c*a |
| | (modified) | |
| 463 | Antisense 5′-3′ | a*G*gauGaGUuauuCuGggacg*a*c |
| | (modified) | |
| 464 | Antisense 5′-3′ | u*C*cggAgGGcucuGgUcagcg*c*u |
| | (modified) | |
| 465 | Antisense 5′-3′ | c*U*ccgGaGGgcucUgGucagc*g*c |
| | (modified) | |
| 466 | Antisense 5′-3′ | u*C*uccGgAGggcuCuGgucag*c*g |
| | (modified) | |
| 467 | Antisense 5′-3′ | g*U*cucCgGAgggcUcUgguca*g*c |
| | (modified) | |
| 468 | Antisense 5′-3′ | a*G*ucuCcGGagggCuCugguc*a*g |
| | (modified) | |
| 469 | Antisense 5′-3′ | u*A*gucUcCGgaggGcUcuggu*c*a |
| | (modified) | |
| 470 | Antisense 5′-3′ | a*U*gacCuCCucccCaUucccu*g*g |
| | (modified) | |
| 471 | Antisense 5′-3′ | u*A*gcaAaGCugauGaCcuccu*c*c |
| | (modified) | |
| 472 | Antisense 5′-3′ | a*G*uagCaAAgcugAuGaccuc*c*u |
| | (modified) | |
| 473 | Antisense 5′-3′ | a*G*ucuGuGAcaguAgCaaagc*u*g |
| | (modified) | |
| 474 | Antisense 5′-3′ | a*U*gguAcAGguggUgGgaccg*a*g |
| | (modified) | |
| 475 | Antisense 5′-3′ | c*A*uggUaCAggugGuGggacc*g*a |
| | (modified) | |
| 476 | Antisense 5′-3′ | g*C*augGuACagguGgUgggac*c*g |
| | (modified) | |
| 477 | Antisense 5′-3′ | g*G*cauGgUAcaggUgGuggga*c*c |
| | (modified) | |
| 478 | Antisense 5′-3′ | g*G*gcaUgGUacagGuGguggg*a*c |
| | (modified) | |
| 479 | Antisense 5′-3′ | c*G*ggcAuGGuacaGgUggugg*g*a |
| | (modified) | |
| 480 | Antisense 5′-3′ | g*C*gggCaUGguacAgGuggug*g*g |
| | (modified) | |
| 481 | Antisense 5′-3′ | g*G*cggGcAUgguaCaGguggu*g*g |
| | (modified) | |
| 482 | Antisense 5′-3′ | a*G*gcgGgCAugguAcAggugg*u*g |
| | (modified) | |
| 483 | Antisense 5′-3′ | c*A*ggcGgGCauggUaCaggug*g*u |
| | (modified) | |
| 484 | Antisense 5′-3′ | a*A*agaGuGCcaggAaGggugg*g*g |
| | (modified) | |
| 485 | Antisense 5′-3′ | a*G*caaAgAGugccAgGaaggg*u*g |
| | (modified) | |
| 486 | Antisense 5′-3′ | a*U*ccuCaAGcaaaGaGugcca*g*g |
| | (modified) | |
| 487 | Antisense 5′-3′ | a*G*aucCuCAagcaAaGagugc*c*a |
| | (modified) | |
| 488 | Antisense 5′-3′ | a*A*gauCcUCaagcAaAgagug*c*c |
| | (modified) | |
| 489 | Antisense 5′-3′ | u*C*ucaCcCCucaaGcCacuag*a*g |
| | (modified) | |
| 490 | Antisense 5′-3′ | a*G*acuUcUCacccCuCaagcc*a*c |
| | (modified) | |
| 491 | Antisense 5′-3′ | a*G*uuuCaGGacacCaGacuuc*u*c |
| | (modified) | |
| 492 | Antisense 5′-3′ | a*G*uugCaGUuucaGgAcacca*g*a |
| | (modified) | |
| 493 | Antisense 5′-3′ | u*A*guuGcAGuuucAgGacacc*a*g |
| | (modified) | |
| 494 | Antisense 5′-3′ | C*U*uaaGcUCuaggAaGggcug*c*u |
| | (modified) | |
| 495 | Antisense 5′-3′ | a*U*cuuAaGCucuaGgAagggc*u*g |
| | (modified) | |
| 496 | Antisense 5′-3′ | a*U*uggCuCGgaucUuAagcuc*u*a |
| | (modified) | |
| 497 | Antisense 5′-3′ | u*C*cugGaAGucuaCgUaaugg*u*c |
| | (modified) | |
| 498 | Antisense 5′-3′ | a*U*ucaGcUGguacCcCucggg*c*u |
| | (modified) | |
| 499 | Antisense 5′-3′ | u*A*auuCaGCugguAcCccucg*g*g |
| | (modified) | |
| 500 | Antisense 5′-3′ | u*G*cagUaAUucagCuGguacc*c*c |
| | (modified) | |
| 501 | Antisense 5′-3′ | C*U*gcaGuAAuucaGcUgguac*c*c |
| | (modified) | |
| 502 | Antisense 5′-3′ | a*C*ugcAgUAauucAgCuggua*c*c |
| | (modified) | |
| 503 | Antisense 5′-3′ | C*A*cugCaGUaauuCaGcuggu*a*c |
| | (modified) | |
| 504 | Antisense 5′-3′ | c*C*acuGcAGuaauUcAgcugg*u*a |
| | (modified) | |
| 505 | Antisense 5′-3′ | g*C*ccaCuGCaguaAuUcagcu*g*g |
| | (modified) | |
| 506 | Antisense 5′-3′ | u*G*cccAcUGcaguAaUucagc*u*g |
| | (modified) | |
| 507 | Antisense 5′-3′ | c*U*gccCaCUgcagUaAuucag*c*u |
| | (modified) | |
| 508 | Antisense 5′-3′ | g*G*cacUgCCcacuGcAguaau*u*c |
| | (modified) | |
| 509 | Antisense 5′-3′ | a*U*ggaAaGAggcaGcAaugcc*u*g |
| | (modified) | |
| 510 | Antisense 5′-3′ | a*A*uggAaAGaggcAgCaaugc*c*u |
| | (modified) | |
| 511 | Antisense 5′-3′ | a*A*gacGgCAgaauGgAaagag*g*c |
| | (modified) | |
| 512 | Antisense 5′-3′ | u*G*uugGcUUugagGaGgcuga*a*g |
| | (modified) | |
| 513 | Antisense 5′-3′ | u*U*guuGgCUuugaGgAggcug*a*a |
| | (modified) | |
| 514 | Antisense 5′-3′ | a*A*ggaUuGUuggcUuUgagga*g*g |
| | (modified) | |
| 515 | Antisense 5′-3′ | a*U*gauCcAGguagAgGagaga*g*a |
| | (modified) | |
| 516 | Antisense 5′-3′ | u*U*augAuCCagguAgAggaga*g*a |
| | (modified) | |
| 517 | Antisense 5′-3′ | a*U*uauGaUCcaggUaGaggag*a*g |
| | (modified) | |
| 518 | Antisense 5′-3′ | a*U*ugcCaUUaugaUcCaggua*g*a |
| | (modified) | |
| 519 | Antisense 5′-3′ | a*C*auuGcCAuuauGaUccagg*u*a |
| | (modified) | |
| 520 | Antisense 5′-3′ | u*G*accAcAUugccAuUaugau*c*c |
| | (modified) | |
| 521 | Antisense 5′-3′ | u*U*gacCaCAuugcCaUuauga*u*c |
| | (modified) | |
| 522 | Antisense 5′-3′ | c*U*ugaCcACauugCcAuuaug*a*u |
| | (modified) | |
| 523 | Antisense 5′-3′ | u*C*uugAcCAcauuGcCauuau*g*a |
| | (modified) | |
| 524 | Antisense 5′-3′ | g*U*cuuGaCCacauUgCcauua*u*g |
| | (modified) | |
| 525 | Antisense 5′-3′ | c*G*ucuUgACcacaUuGccauu*a*u |
| | (modified) | |
| 526 | Antisense 5′-3′ | a*C*aucCgUCuugaCcAcauug*c*c |
| | (modified) | |
| 527 | Antisense 5′-3′ | a*U*aucUgGCacauCcGucuug*a*c |
| | (modified) | |
| 528 | Antisense 5′-3′ | c*U*agcUgCAgccaCaGgccuc*c*a |
| | (modified) | |
| 529 | Antisense 5′-3′ | g*C*uagCuGCagccAcAggccu*c*c |
| | (modified) | |
| 530 | Antisense 5′-3′ | u*G*cuaGcUGcagcCaCaggcc*u*c |
| | (modified) | |
| 531 | Antisense 5′-3′ | u*U*gcuAgCUgcagCcAcaggc*c*u |
| | (modified) | |
| 532 | Antisense 5′-3′ | c*U*ugcUaGCugcaGcCacagg*c*c |
| | (modified) | |
| 533 | Antisense 5′-3′ | u*U*gguCuCUucacUcCaaagc*c*c |
| | (modified) | |
| 534 | Antisense 5′-3′ | a*G*ucaUaUUgccaGgUgguug*u*u |
| | (modified) | |
| 535 | Antisense 5′-3′ | u*G*aguCaUAuugcCaGguggu*u*g |
| | (modified) | |
| 536 | Antisense 5′-3′ | a*G*ugaGuCAuauuGcCaggug*g*u |
| | (modified) | |
| 537 | Antisense 5′-3′ | a*G*aaaGuGCccauUuGggucc*c*a |
| | (modified) | |
| 538 | Antisense 5′-3′ | a*C*aagAaAGugccCaUuuggg*u*c |
| | (modified) | |
| 539 | Antisense 5′-3′ | a*G*acaAgAAagugCcCauuug*g*g |
| | (modified) | |
| 540 | Antisense 5′-3′ | a*G*ucuCaGAcaagAaAgugcc*c*a |
| | (modified) | |
| 541 | Antisense 5′-3′ | a*G*aguCuCAgacaAgAaagug*c*c |
| | (modified) | |
| 542 | Antisense 5′-3′ | u*A*agcCaGAgucuCaGacaag*a*a |
| | (modified) | |
| 543 | Antisense 5′-3′ | u*A*auuUuUCucugCcUucccu*c*c |
| | (modified) | |
| 544 | Antisense 5′-3′ | g*U*aauUuUUcucuGcCuuccc*u*c |
| | (modified) | |
| 545 | Antisense 5′-3′ | u*U*ucuCaUCuuggGaGaggcu*a*a |
| | (modified) | |
| 546 | Antisense 5′-3′ | a*C*uuuCuCAucuuGgGagagg*c*u |
| | (modified) | |
| 547 | Antisense 5′-3′ | a*G*gacUuUCucauCuUgggag*a*g |
| | (modified) | |
| 548 | Antisense 5′-3′ | u*C*uauCuGCuuccUcCucccc*u*c |
| | (modified) | |
| 549 | Antisense 5′-3′ | a*U*cuaUcUGcuucCuCcuccc*c*u |
| | (modified) | |
| 550 | Antisense 5′-3′ | u*U*uucCuGAcuccUgUuucug*g*g |
| | (modified) | |
| 551 | Antisense 5′-3′ | g*G*aacUuCUuaggCuUagugc*c*u |
| | (modified) | |
| 552 | Antisense 5′-3′ | a*A*augCuUGucucCcAguggg*u*c |
| | (modified) | |
| 553 | Antisense 5′-3′ | u*A*aauGcUUgucuCcCagugg*g*u |
| | (modified) | |
| 554 | Antisense 5′-3′ | u*A*uaaAuGCuuguCuCccagu*g*g |
| | (modified) | |
| 555 | Antisense 5′-3′ | a*G*uauAaAUgcuuGuCuccca*g*u |
| | (modified) | |
| 556 | Antisense 5′-3′ | a*A*guaUaAAugcuUgUcuccc*a*g |
| | (modified) | |
| 557 | Antisense 5′-3′ | a*A*gaaAgUAuaaaUgCuuguc*u*c |
| | (modified) | |
| 558 | Antisense 5′-3′ | a*U*aagCcAGgcgcGgUggcuc*a*c |
| | (modified) | |
| 559 | Antisense 5′-3′ | a*G*uauAaGCcaggCgCggugg*c*u |
| | (modified) | |
| 560 | Antisense 5′-3′ | a*A*aguAuAAgccaGgCgcggu*g*g |
| | (modified) | |
| 561 | Antisense 5′-3′ | a*G*aaaGuAUaagcCaGgcgcg*g*u |
| | (modified) | |
| 562 | Antisense 5′-3′ | a*A*gaaAgUAuaagCcAggcgc*g*g |
| | (modified) | |
| 563 | Antisense 5′-3′ | u*A*agaAaGUauaaGcCaggcg*c*g |
| | (modified) | |
| 564 | Antisense 5′-3′ | u*U*aagAaAGuauaAgCcaggc*g*c |
| | (modified) | |
| 565 | Antisense 5′-3′ | a*U*uaaGaAAguauAaGccagg*c*g |
| | (modified) | |
| 566 | Antisense 5′-3′ | u*U*auuAaGAaaguAuAagcca*g*g |
| | (modified) | |
| 567 | Antisense 5′-3′ | u*U*uauUaAGaaagUaUaagcc*a*g |
| | (modified) | |
| 568 | Antisense 5′-3′ | u*U*uuuAuUAagaaAgUauaag*c*c |
| | (modified) | |
| 569 | Antisense 5′-3′ | u*U*ugaCuUUguggAcAccccu*g*a |
| | (modified) | |
| 570 | Antisense 5′-3′ | c*U*uugAcUUugugGaCacccc*u*g |
| | (modified) | |
| 571 | Antisense 5′-3′ | a*A*cauGuUAguauUaUuauga*a*a |
| | (modified) | |
| 572 | Antisense 5′-3′ | a*A*aggCaAAuaacAuGuuagu*a*u |
| | (modified) | |
| 573 | Antisense 5′-3′ | a*G*aauUcAAaaggCaAauaac*a*u |
| | (modified) | |
| 574 | Antisense 5′-3′ | a*U*aauGaGAauucAaAaggca*a*a |
| | (modified) | |
| 575 | Antisense 5′-3′ | g*A*uaaUgAGaauuCaAaaggc*a*a |
| | (modified) | |
| 576 | Antisense 5′-3′ | a*G*auaAuGAgaauUcAaaagg*c*a |
| | (modified) | |
| 577 | Antisense 5′-3′ | a*A*gauAaUGagaaUuCaaaag*g*c |
| | (modified) | |
| 578 | Antisense 5′-3′ | u*U*uuaAgAUaaugAgAauuca*a*a |
| | (modified) | |
| 579 | Antisense 5′-3′ | a*U*uuuAaGAuaauGaGaauuc*a*a |
| | (modified) | |
| 580 | Antisense 5′-3′ | a*U*guaAuCAcaugUcAcacgg*c*c |
| | (modified) | |
| 581 | Antisense 5′-3′ | a*U*gauGuAAucacAuGucaca*c*g |
| | (modified) | |
| 582 | Antisense 5′-3′ | a*G*augAuGUaaucAcAuguca*c*a |
| | (modified) | |
| 583 | Antisense 5′-3′ | a*A*gauGaUGuaauCaCauguc*a*c |
| | (modified) | |
| 584 | Antisense 5′-3′ | u*U*aacAaUGauguCaGaaaga*u*g |
| | (modified) | |
| 585 | Antisense 5′-3′ | a*U*uaaCaAUgaugUcAgaaag*a*u |
| | (modified) | |
| 586 | Antisense 5′-3′ | C*A*uucCaUUaacaAuGauguc*a*g |
| | (modified) | |
| 587 | Antisense 5′-3′ | a*C*aagCaCAcauuCcAuuaac*a*a |
| | (modified) | |
| 588 | >NM_031479.5 | AGTAGCCAGACATGAGCTGTGAGGGTC |
| | Homo sapiens | AAGCACAGCTATCCATCAGATGATCTA |
| | inhibin | CTTTCAGCCTTCCTGAGTCCCAGACAA |
| | subunit beta E | TAGAAGACAGGTGGCTGTACCCTTGGC |
| | (INHBE), mRNA | CAAGGGTAGGTGTGGCAGTGGTGTCTG |
| | | CTGTCACTGTGCCCTCATTGGCCCCCA |
| | | GCAATCAGACTCAACAGACGGAGCAAC |
| | | TGCCATCCGAGGCTCCTGAACCAGGGC |
| | | CATTCACCAGGAGCATGCGGCTCCCTG |
| | | ATGTCCAGCTCTGGCTGGTGCTGCTGT |
| | | GGGCACTGGTGCGAGCACAGGGGACAG |
| | | GGTCTGTGTGTCCCTCCTGTGGGGGCT |
| | | CCAAACTGGCACCCCAAGCAGAACGAG |
| | | CTCTGGTGCTGGAGCTAGCCAAGCAGC |
| | | AAATCCTGGATGGGTTGCACCTGACCA |
| | | GTCGTCCCAGAATAACTCATCCTCCAC |
| | | CCCAGGCAGCGCTGACCAGAGCCCTCC |
| | | GGAGACTACAGCCAGGGAGTGTGGCTC |
| | | CAGGGAATGGGGAGGAGGTCATCAGCT |
| | | TTGCTACTGTCACAGACTCCACTTCAG |
| | | CCTACAGCTCCCTGCTCACTTTTCACC |
| | | TGTCCACTCCTCGGTCCCACCACCTGT |
| | | ACCATGCCCGCCTGTGGCTGCACGTGC |
| | | TCCCCACCCTTCCTGGCACTCTTTGCT |
| | | TGAGGATCTTCCGATGGGGACCAAGGA |
| | | GGAGGCGCCAAGGGTCCCGCACTCTCC |
| | | TGGCTGAGCACCACATCACCAACCTGG |
| | | GCTGGCATACCTTAACTCTGCCCTCTA |
| | | GTGGCTTGAGGGGTGAGAAGTCTGGTG |
| | | TCCTGAAACTGCAACTAGACTGCAGAC |
| | | CCCTAGAAGGCAACAGCACAGTTACTG |
| | | GACAACCGAGGCGGCTCTTGGACACAG |
| | | CAGGACACCAGCAGCCCTTCCTAGAGC |
| | | TTAAGATCCGAGCCAATGAGCCTGGAG |
| | | CAGGCCGGGCCAGGAGGAGGACCCCCA |
| | | CCTGTGAGCCTGCGACCCCCTTATGTT |
| | | GCAGGCGAGACCATTACGTAGACTTCC |
| | | AGGAACTGGGATGGCGGGACTGGATAC |
| | | TGCAGCCCGAGGGGTACCAGCTGAATT |
| | | ACTGCAGTGGGCAGTGCCCTCCCCACC |
| | | TGGCTGGCAGCCCAGGCATTGCTGCCT |
| | | CTTTCCATTCTGCCGTCTTCAGCCTCC |
| | | TCAAAGCCAACAATCCTTGGCCTGCCA |
| | | GTACCTCCTGTTGTGTCCCTACTGCCC |
| | | GAAGGCCCCTCTCTCTCCTCTACCTGG |
| | | ATCATAATGGCAATGTGGTCAAGACGG |
| | | ATGTGCCAGATATGGTGGTGGAGGCCT |
| | | GTGGCTGCAGCTAGCAAGAGGACCTGG |
| | | GGCTTTGGAGTGAAGAGACCAAGATGA |
| | | AGTTTCCCAGGCACAGGGCATCTGTGA |
| | | CTGGAGGCATCAGATTCCTGATCCACA |
| | | CCCCAACCCAACAACCACCTGGCAATA |
| | | TGACTCACTTGACCCCTATGGGACCCA |
| | | AATGGGCACTTTCTTGTCTGAGACTCT |
| | | GGCTTATTCCAGGTTGGCTGATGTGTT |
| | | GGGAGATGGGTAAAGCGTTTCTTCTAA |
| | | AGGGGTCTACCCAGAAAGCATGATTTC |
| | | CTGCCCTAAGTCCTGTGAGAAGATGTC |
| | | AGGGACTAGGGAGGGAGGGAGGGAAGG |
| | | CAGAGAAAAATTACTTAGCCTCTCCCA |
| | | AGATGAGAAAGTCCTCAAGTGAGGGGA |
| | | GGAGGAAGCAGATAGATGGTCCAGCAG |
| | | GCTTGAAGCAGGGTAAGCAGGCTGGCC |
| | | CAGGGTAAGGGCTGTTGAGGTACCTTA |
| | | AGGGAAGGTCAAGAGGGAGATGGGCAA |
| | | GGCGCTGAGGGAGGATGCTTAGGGGAC |
| | | CCCCAGAAACAGGAGTCAGGAAAATGA |
| | | GGCACTAAGCCTAAGAAGTTCCCTGGT |
| | | TTTTCCCAGGGGACAGGACCCACTGGG |
| | | AGACAAGCATTTATACTTTCTTTCTTC |
| | | TTTTTTATTTTTTTGAGATCGAGTCTC |
| | | GCTCTGTCACCAGGCTGGAGTGCAGTG |
| | | ACACGATCTTGGCTCACTGCAACCTCC |
| | | GTCTCCTGGGTTCAAGTGATTCTTCTG |
| | | CCTCAGCCTCCCGAGCAGCTGGGATTA |
| | | CAGGCGCCCACTAATTTTTGTATTCTT |
| | | AGTAGAAACGAGGTTTCAACATGTTGG |
| | | CCAGGATGGTCTCAATCTCTTGACCTC |
| | | TTGATCCACCCGACTTGGCCTCCCGAA |
| | | GTGATGAGATTATAGGCGTGAGCCACC |
| | | GCGCCTGGCTTATACTTTCTTAATAAA |
| | | AAGGAGAAAGAAAATCAACAAATGTGA |
| | | GTCATAAAGAAGGGTTAGGGTGATGGT |
| | | CCAGAGCAACAGTTCTTCAAGTGTACT |
| | | CTGTAGGCTTCTGGGAGGTCCCTTTTC |
| | | AGGGGTGTCCACAAAGTCAAAGCTATT |
| | | TTCATAATAATACTAACATGTTATTTG |
| | | CCTTTTGAATTCTCATTATCTTAAAAT |
| | | TGTATTGTGGAGTTTTCCAGAGGCCGT |
| | | GTGACATGTGATTACATCATCTTTCTG |
| | | ACATCATTGTTAATGGAATGTGTGCTT |
| | | GTA |
| 589 | Sense 5′-3′ | ACUCUUUGCUUGAGGAUCUU |
| 590 | Sense 5′-3′ | ACUCUUUGCUUGAGGAUCUU |
| 591 | Sense 5′-3′ | ACAAGCAUUUAUACUUUCUU |
| 592 | Sense 5′-3′ | ACAAGCAUUUAUACUUUCUU |
| 593 | Sense 5′-3′ | GGCUUAUACUUUCUUAAUAA |
| 594 | Sense 5′-3′ | TGGCUUAUACUUUCUUAAUAAUU |
| 595 | Sense 5′-3′ | ACAAGCAUUUAUACUUUCUUU |
| 596 | Sense 5′-3′ | UGAAGAGACCAAGAUGAAGUU |
| 597 | Sense 5′-3′ | AGAGACCAAGAUGAAGUUUCC |
| 598 | Antisense 5′-3′ | AAGAUCCUCAAGCAAAGAGUGCC |
| 599 | Antisense 5′-3′ | AAGAUCCUCAAGCAAAGAGUGCC |
| 600 | Antisense 5′-3′ | AAGAAAGUAUAAAUGCUUGUCUC |
| 601 | Antisense 5′-3′ | AAGAAAGUAUAAAUGCUUGUCUC |
| 602 | Antisense 5′-3′ | UUAUUAAGAAAGUAUAAGCCAGG |
| 603 | Antisense 5′-3′ | UUAUUAAGAAAGUAUAAGCCAGG |
| 604 | Antisense 5′-3′ | UAAGAAAGUAUAAAUGCUUGUCU |
| 605 | Antisense 5′-3′ | UACUUCAUCUUGGUCUCUUCACU |
| 606 | Antisense 5′-3′ | UGAAACUUCAUCUUGGUCUCUUC |
| 607 | Sense 5′-3′ | a*c*ucuUuGCUuGaggaucuu |
| | (modified) | |
| 608 | Sense 5′-3′ | a*c*ucuUuGCUuGagga |
| | (modified) | (utU) cu (tmU) |
| 609 | Sense 5′-3′ | a*c*aagCaUUUaUacuuucuu |
| | (modified) | |
| 610 | Sense 5′-3′ | a*c*aagCaUUUaUacuuucu |
| | (modified) | (tmU) |
| 611 | Sense 5′-3′ | g*g*cuuAuACUuUcuuaauaa |
| | (modified) | |
| 612 | Sense 5′-3′ | invdT*g*g*cuuAuACUuUcuua |
| | (modified) | (utA) uaa*u* (tmU) |
| 613 | Sense 5′-3′ | a*c*aagcauUUAuacuuuuuu |
| | (modified) | |
| 614 | Sense 5′-3′ | u*g*aagagaCCAagaugaaguu |
| | (modified) | |
| 615 | Sense 5′-3′ | a*g*agaccaAGAugaaguuucc |
| | (modified) | |
| 616 | Antisense 5′-3′ | va*A*gAuCcUcaagcAaAgagug |
| | (modified) | *c*c |
| 617 | Antisense 5′-3′ | va*A*gAuCcUcaagcAaAgagug |
| | (modified) | *c*c |
| 618 | Antisense 5′-3′ | va*A*gaaAgUauaaaUgcuuguc |
| | (modified) | *u*c |
| 619 | Antisense 5′-3′ | va*A*gaaAgUauaaaUgcuuguc |
| | (modified) | *u*c |
| 620 | Antisense 5′-3′ | vu*U*aUuAaGaaaguAuAagcca |
| | (modified) | *g*g |
| 621 | Antisense 5′-3′ | vu*U*aUuAaGaaaguAuAagcca |
| | (modified) | *g*g |
| 622 | Antisense 5′-3′ | vu*A*aGaAaGuauaaAuGcuugucu |
| | (modified) | |
| 623 | Antisense 5′-3′ | vu*A*cUuCaUcuuggUcUcuucacu |
| | (modified) | |
| 624 | Antisense 5′-3′ | vu*G*aAaCuUcaucuuggucucuuc |
| | (modified) | |
| 625 | Sense 5′-3′ | invdT*a*c*ucuUuGCUuGaggauc |
| | (modified) | uu*u*u |
| 626 | Sense 5′-3′ | invdT*a*c*ucuUuGCUuGagga |
| | (modified) | (utU) cuu*u* (tmU) |
| 627 | Sense 5′-3′ | invdT*a*c*aagCaUUUaUacuuu |
| | (modified) | cuu*u*u |
| 628 | Sense 5′-3′ | invdT*a*c*aagCaUUUaUacuu |
| | (modified) | (tmU) cuu*u* (tmU) |
| 629 | Sense 5′-3′ | invdT*g*g*cuuAuACUuUcuuaa |
| | (modified) | uaa*u*u |
| 630 | Sense 5′-3′ | g*g*cuuAuACUuUcuuaauaa |
| | (modified) | |
| 631 | Sense 5′-3′ | C*u*gucaCaGACuccacuucau |
| | (modified) | (L96) |
| 632 | Antisense 5′-3′ | va*A*gAuCcUcaagcAaAgagug |
| | (modified) | *c*c |
| 633 | Antisense 5′-3′ | va*A*gAuCcUcaagcAaAgagug |
| | (modified) | *c*c |
| 634 | Antisense 5′-3′ | va*A*gaaAgUauaaaUgcuuguc |
| | (modified) | *u*c |
| 635 | Antisense 5′-3′ | va*A*gaaAgUauaaaUgcuuguc |
| | (modified) | *u*c |
| 636 | Antisense 5′-3′ | vu*U*aUuAaGaaaguAuAagcca |
| | (modified) | *g*g |
| 637 | Antisense 5′-3′ | vu*U*aUuAaGaaaguAuAag |
| | (modified) | (tmC) ca*g*g |
| 638 | Antisense 5′-3′ | A*U*gadAg (Tgn) |
| | (modified) | ggagucUgUgacag*u*a |
| 639 | Sense 5′-3′ | TACUCUUUGCUUGAGGAUCUUUU |
| 640 | Sense 5′-3′ | TACUCUUUGCUUGAGGAUCUUUU |
| 641 | Sense 5′-3′ | TACAAGCAUUUAUACUUUCUUUU |
| 642 | Sense 5′-3′ | TACAAGCAUUUAUACUUUCUUUU |
| 643 | Sense 5′-3′ | TGGCUUAUACUUUCUUAAUAAUU |
| 644 | Sense 5′-3′ | GGCUUAUACUUUCUUAAUAA |
| 645 | Sense 5′-3′ | CUGUCACAGACUCCACUUCAU |
| 646 | Antisense 5′-3′ | AAGAUCCUCAAGCAAAGAGUGCC |
| 647 | Antisense 5′-3′ | AAGAUCCUCAAGCAAAGAGUGCC |
| 648 | Antisense 5′-3′ | AAGAAAGUAUAAAUGCUUGUCUC |
| 649 | Antisense 5′-3′ | AAGAAAGUAUAAAUGCUUGUCUC |
| 650 | Antisense 5′-3′ | UUAUUAAGAAAGUAUAAGCCAGG |
| 651 | Antisense 5′-3′ | UUAUUAAGAAAGUAUAAGCCAGG |
| 652 | Antisense 5′-3′ | AUGAAGTGGAGUCUGUGACAGUA |
| 653 | Sense 5′-3′ | u*g*gcuuAuACUuucuuaauaa |
| | (modified) | |
| 654 | Sense 5′-3′ | u*u*ggagugAAGagaccaagau |
| | (modified) | |
| 655 | Sense 5′-3′ | u*c*uuuccaUUCugccgucuuc |
| | (modified) | |
| 656 | Sense 5′-3′ | g*g*agacaaGCAuuuauacuuu |
| | (modified) | |
| 657 | Sense 5′-3′ | g*a*gacaagCAUuuauacuuuc |
| | (modified) | |
| 658 | Sense 5′-3′ | a*g*agaccaAGAugaaguuucc |
| | (modified) | |
| 659 | Sense 5′-3′ | a*a*gaaguuCCCugguuuuucc |
| | (modified) | |
| 660 | Antisense 5′-3′ | u*U*auuAaGAaaguAuAagcca |
| | (modified) | *g*g |
| 661 | Antisense 5′-3′ | vu*U*cUuGgUcucuuCaCuccaaag |
| | (modified) | |
| 662 | Antisense 5′-3′ | vu*A*aGaCgGcagaaUgGaaagagg |
| | (modified) | |
| 663 | Antisense 5′-3′ | vu*A*aGuAuAaaugcUuGucuccca |
| | (modified) | |
| 664 | Antisense 5′-3′ | vu*A*aAgUaUaaaugCuUgucuccc |
| | (modified) | |
| 665 | Antisense 5′-3′ | vu*G*aAaCuUcaucuuggucucuuc |
| | (modified) | |
| 666 | Antisense 5′-3′ | vu*G*aAaAaCcagggAaCuucuuag |
| | (modified) | |
| 667 | Sense 5′-3′ | UGGCUUAUACUUUCUUAAUAA |
| 668 | Sense 5′-3′ | UUGGAGUGAAGAGACCAAGAU |
| 670 | Sense 5′-3′ | UCUUUCCAUUCUGCCGUCUUC |
| 671 | Sense 5′-3′ | GGAGACAAGCAUUUAUACUUU |
| 672 | Sense 5′-3′ | GAGACAAGCAUUUAUACUUUC |
| 673 | Sense 5′-3′ | AGAGACCAAGAUGAAGUUUCC |
| 674 | Sense 5′-3′ | AAGAAGUUCCCUGGUUUUUCC |
| 675 | Antisense 5′-3′ | UUAUUAAGAAAGUAUAAGCCAGG |
| 676 | Antisense 5′-3′ | UUCUUGGUCUCUUCACUCCAAAG |
| 677 | Antisense 5′-3′ | UAAGACGGCAGAAUGGAAAGAGG |
| 678 | Antisense 5′-3′ | UAAGUAUAAAUGCUUGUCUCCCA |
| 679 | Antisense 5′-3′ | UAAAGUAUAAAUGCUUGUCUCCC |
| 680 | Antisense 5′-3′ | UGAAACUUCAUCUUGGUCUCUUC |
| 681 | Antisense 5′-3′ | UGAAAAACCAGGGAACUUCUUAG |

### INCORPORATION BY REFERENCE

The entire disclosure of each of the patent and scientific documents referred to herein is incorporated by reference for all purposes.

### EQUIVALENTS

The disclosure can be embodied in other specific forms without departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

1-81. (canceled)

82. A double-stranded ribonucleic acid (dsRNA), wherein the dsRNA comprises a sense strand and an antisense strand each 18 to 25 nucleotides in length, wherein:
(a) the antisense strand comprises a nucleobase sequence of at least 15 contiguous nucleotides ofAAGACGGCAGAAUGGAAAGAGGC (SEQ ID NO: 217); and
(b) the sense strand comprises a nucleobase sequence of at least 15 contiguous nucleotides of CUCUUUCCAUUCUGCCGUCUU (SEQ ID NO: 71);
wherein at least one nucleotide of the dsRNA is a modified nucleotide selected from a 2′-O-methyl modified nucleotide or a 2′-fluoro modified nucleotide;
wherein at least one internucleoside linkage is a phosphorothioate linkage; and
wherein the dsRNA further comprises a ligand or targeting moiety that comprises at least one N-acetyl-galactosamine (GalNAc).

83. The dsRNA of claim 82, wherein the antisense strand comprises a nucleobase sequence of 17 contiguous nucleotides of AAGACGGCAGAAUGGAAAGAGGC (SEQ ID NO: 217).

84. The dsRNA of claim 82, wherein the sense strand comprises a nucleobase sequence of 17 contiguous nucleotides of CUCUUUCCAUUCUGCCGUCUU (SEQ ID NO: 71).

85. The dsRNA of claim 82, wherein the antisense strand is 23 nucleotides in length and the sense strand is 21 nucleotides in length.

86. The dsRNA of claim 82, wherein the antisense strand comprises a nucleobase sequence of 17 contiguous nucleotides of AAGACGGCAGAAUGGAAAGAGGC (SEQ ID NO: 217), the sense strand comprises a nucleobase sequence of 17 contiguous nucleotides of CUCUUUCCAUUCUGCCGUCUU (SEQ ID NO: 71), the antisense strand is 23 nucleotides in length, and the sense strand is 21 nucleotides in length.

87. The dsRNA of claim 82, wherein the antisense strand has a 3′ end nucleotide overhang compared to the sense strand.

88. The dsRNA of claim 82, wherein the antisense strand and the sense strand each comprise at least one modified nucleotide.

89. The dsRNA of claim 82, wherein the antisense strand and the sense strand each comprise at least one phosphorothioate linkage.

90. The dsRNA of claim 82, wherein the ligand or targeting moiety is conjugated to the 5′ end of the sense strand of the dsRNA.

91. The dsRNA of claim 86, wherein the ligand or targeting moiety is conjugated to the 5′ end of the sense strand of the dsRNA.

92. A double-stranded ribonucleic acid (dsRNA), wherein the dsRNA comprises an antisense strand of 23 nucleotides in length and a sense strand of 21 nucleotides in length, wherein:
(a) the antisense strand comprises a nucleobase sequence of 17 contiguous nucleotides ofAAGACGGCAGAAUGGAAAGAGGC (SEQ ID NO: 217); and
(b) the sense strand comprises a nucleobase sequence of 17 contiguous nucleotides of CUCUUUCCAUUCUGCCGUCUU (SEQ ID NO: 71);
wherein the antisense strand has a 3′ end nucleotide overhang compared to the sense strand;
wherein the antisense strand and the sense strand each comprise at least one modified nucleotide selected from a 2′-O-methyl modified nucleotide or a 2′-fluoro modified nucleotide;
wherein the antisense strand and the sense strand each comprise at least one phosphorothioate linkage; and
wherein the dsRNA further comprises a ligand or targeting moiety that comprises at least one N-acetyl-galactosamine (GalNAc), wherein the ligand or targeting moiety is conjugated to the 5′ end of the sense strand of the dsRNA.

93. A pharmaceutical composition comprising the dsRNA of claim 82, and a pharmaceutically acceptable carrier, diluent, excipient, or combination thereof.

94. A pharmaceutical composition comprising the dsRNA of claim 92, and a pharmaceutically acceptable carrier, diluent, excipient, or combination thereof.

95. A method of inhibiting Inhibin Subunit Beta E (INHBE) expression in a cell, the method comprising: (a) contacting the cell with the dsRNA of claim 82; and (b) maintaining the cell produced in step (a) for a time sufficient to obtain degradation of the mRNA transcript of an INHBE gene, thereby inhibiting expression of the INHBE gene in the cell.

96. A method of inhibiting Inhibin Subunit Beta E (INHBE) expression in a cell, the method comprising: (a) contacting the cell with the dsRNA of claim 92; and (b) maintaining the cell produced in step (a) for a time sufficient to obtain degradation of the mRNA transcript of an INHBE gene, thereby inhibiting expression of the INHBE gene in the cell.

97. A method of treating a disorder mediated by or associated with Inhibin Subunit Beta E (INHBE) comprising administering to a subject in need of such treatment a therapeutically effective amount of the dsRNA of claim 82.

98. A method of treating a disorder mediated by or associated with Inhibin Subunit Beta E (INHBE) comprising administering to a subject in need of such treatment a therapeutically effective amount of the dsRNA of claim 92.
